SEQUENCE LISTING (380 sequences)

SEQ ID NO: 1 - DNA, 21 bp, Homo sapiens
cagccacagaatattatgtaa

SEQ ID NO: 2 - DNA, 21 bp, Homo sapiens
ctggatgtttatttataaaga

SEQ ID NO: 3 - DNA, 21 bp, Homo sapiens
tagaattgtcatttctcttta

SEQ ID NO: 4 - DNA, 21 bp, Homo sapiens
tcccagctatctataacctta

SEQ ID NO: 5 - DNA, 21 bp, Homo sapiens
cacgtactgtttcacgacaat

SEQ ID NO: 6 - DNA, 21 bp, Homo sapiens
gtgctgaaacttagaaatata

SEQ ID NO: 7 - DNA, 21 bp, Homo sapiens
agggaatatatctctatttga

SEQ ID NO: 8 - DNA, 21 bp, Homo sapiens
acacagcgccatagtagttaa

SEQ ID NO: 9 - DNA, 21 bp, Homo sapiens
acagatgtgtgagaacaagaa

SEQ ID NO: 10 - DNA, 21 bp, Homo sapiens
atgacagatgtgtgagaacaa

SEQ ID NO: 11 - DNA, 21 bp, Homo sapiens
atggctcttccttgagtgaaa

SEQ ID NO: 12 - DNA, 21 bp, Homo sapiens
ctgcccaactgtgaccaacta

SEQ ID NO: 13 - DNA, 21 bp, Homo sapiens
ccgggacgagtggaatgatta

SEQ ID NO: 14 - DNA, 21 bp, Homo sapiens
tccggtgtgctcacagcttta

SEQ ID NO: 15 - DNA, 21 bp, Homo sapiens
caggtccctgttcctgctata

SEQ ID NO: 16 - DNA, 21 bp, Homo sapiens
cgggacgagtggaatgattaa

SEQ ID NO: 17 - DNA, 21 bp, Homo sapiens
cctgaactttacgatcctgaa

SEQ ID NO: 18 - DNA, 21 bp, Homo sapiens
aagataaacagtccacttcaa

SEQ ID NO: 19 - DNA, 21 bp, Homo sapiens
caggcccttggtagatgtatt

SEQ ID NO: 20 - DNA, 21 bp, Homo sapiens
tagcatggcaacaatctctta

SEQ ID NO: 21 - DNA, 21 bp, Homo sapiens
aaccttctccatgcaaatcta

SEQ ID NO: 22 - DNA, 21 bp, Homo sapiens
cccaatttgcttgaatgggaa

SEQ ID NO: 23 - DNA, 21 bp, Homo sapiens
ctgcttatttggtgtggttaa

SEQ ID NO: 24 - DNA, 21 bp, Homo sapiens
aagtgttatatccctaaccaa

SEQ ID NO: 25 - DNA, 21 bp, Homo sapiens
aaggccagtatgatttataaa

SEQ ID NO: 26 - DNA, 21 bp, Homo sapiens
ctcctacgatacgctactata

SEQ ID NO: 27 - DNA, 21 bp, Homo sapiens
agggttatcttagatgtttca

SEQ ID NO: 28 - DNA, 21 bp, Homo sapiens
atgcatgtagtcactttataa

SEQ ID NO: 29 - DNA, 21 bp, Homo sapiens
caccaccatgatgttcgggta

SEQ ID NO: 30 - DNA, 21 bp, Homo sapiens
ctccctaagctggaacagcta

SEQ ID NO: 31 - DNA, 21 bp, Homo sapiens
aaggcatccgctgaagaccaa

SEQ ID NO: 32 - DNA, 21 bp, Homo sapiens
cagacattcacccagcatcaa

SEQ ID NO: 33 - DNA, 21 bp, Homo sapiens
aaggcgctatttggcgcttca

SEQ ID NO: 34 - DNA, 21 bp, Homo sapiens
aagggttatctctttcataca

SEQ ID NO: 35 - DNA, 21 bp, Homo sapiens
cagcttagctagcattactaa

SEQ ID NO: 36 - DNA, 21 bp, Homo sapiens
ctggccaaatagcaaagacaa

SEQ ID NO: 37 - DNA, 21 bp, Homo sapiens
caccgacgagtctcaactaaa

SEQ ID NO: 38 - DNA, 21 bp, Homo sapiens
tcgaggcattacaatcgcgaa

SEQ ID NO: 39 - DNA, 21 bp, Homo sapiens
ttgcctcagagcatacctgaa

SEQ ID NO: 40 - DNA, 21 bp, Homo sapiens
caccttgtttcggactataga

SEQ ID NO: 41 - DNA, 21 bp, Homo sapiens
cacaataaactaaattctgaa

SEQ ID NO: 42 - DNA, 21 bp, Homo sapiens
aaggaagaagagaaacggtta

SEQ ID NO: 43 - DNA, 21 bp, Homo sapiens
ctgccctttaataaagcatta

SEQ ID NO: 44 - DNA, 21 bp, Homo sapiens
cagccatggattgcaaagata

SEQ ID NO: 45 - DNA, 21 bp, Homo sapiens
aggcaggtgtctcatatgtaa

SEQ ID NO: 46 - DNA, 21 bp, Homo sapiens
cagggtttccactagacatta

SEQ ID NO: 47 - DNA, 21 bp, Homo sapiens
atgcgaattactatatataat

SEQ ID NO: 48 - DNA, 21 bp, Homo sapiens
aatgcgaattactatatataa

SEQ ID NO: 49 - DNA, 21 bp, Homo sapiens
aaggtgttaaatacacatgaa

SEQ ID NO: 50 - DNA, 21 bp, Homo sapiens
aatgatgatttcaaacataaa

SEQ ID NO: 51 - DNA, 21 bp, Homo sapiens
aaagataaggttgaccatata

SEQ ID NO: 52 - DNA, 21 bp, Homo sapiens
tggcagcactattgtaattaa

SEQ ID NO: 53 - DNA, 21 bp, Homo sapiens
ccggagcatctcagcaagcaa

SEQ ID NO: 54 - DNA, 21 bp, Homo sapiens
tgccttgaactgggacataaa

SEQ ID NO: 55 - DNA, 21 bp, Homo sapiens
ctggcagaggatggcaccata

SEQ ID NO: 56 - DNA, 21 bp, Homo sapiens
cagcatcatcctcctatcaga

SEQ ID NO: 57 - DNA, 21 bp, Homo sapiens
ttgagacgagggattatttca

SEQ ID NO: 58 - DNA, 21 bp, Homo sapiens
tagggaccaggtagactttaa

SEQ ID NO: 59 - DNA, 21 bp, Homo sapiens
acccattgggaatgagtttaa

SEQ ID NO: 60 - DNA, 21 bp, Homo sapiens
tccgtgctgttggcaacttta

SEQ ID NO: 61 - DNA, 21 bp, Homo sapiens
aggtatcgggctaatcgagaa

SEQ ID NO: 62 - DNA, 21 bp, Homo sapiens
aacgaatggattcctatataa

SEQ ID NO: 63 - DNA, 21 bp, Homo sapiens
acccgggagattgctgacaaa

SEQ ID NO: 64 - DNA, 21 bp, Homo sapiens
gcggatttacgacattaccaa

SEQ ID NO: 65 - DNA, 21 bp, Homo sapiens
cagggtcagaccagtaacaaa

SEQ ID NO: 66 - DNA, 21 bp, Homo sapiens
caggaggaactttctgactta

SEQ ID NO: 67 - DNA, 21 bp, Homo sapiens
cagatcgtcattgcagttaaa

SEQ ID NO: 68 - DNA, 21 bp, Homo sapiens
accacttagattactgagtaa

SEQ ID NO: 69 - DNA, 21 bp, Homo sapiens
gaggatgacatcgccctgcaa

SEQ ID NO: 70 - DNA, 21 bp, Homo sapiens
tcccagtttgcgaattaataa

SEQ ID NO: 71 - DNA, 21 bp, Homo sapiens
tcgttggagactgaagagaaa

SEQ ID NO: 72 - DNA, 21 bp, Homo sapiens
ctgctgtgacaacgacatcaa

SEQ ID NO: 73 - DNA, 21 bp, Homo sapiens
cagcatccctttagctttcaa

SEQ ID NO: 74 - DNA, 21 bp, Homo sapiens
tcggcgcttgcggaagatgaa

SEQ ID NO: 75 - DNA, 21 bp, Homo sapiens
ccaggagaagaagatcaagaa

SEQ ID NO: 76 - DNA, 21 bp, Homo sapiens
cacggagaagatccaggagaa

SEQ ID NO: 77 - DNA, 21 bp, Homo sapiens
aacaattgggtacccagttaa

SEQ ID NO: 78 - DNA, 21 bp, Homo sapiens
ctgcgccaacacagaaattat

SEQ ID NO: 79 - DNA, 21 bp, Homo sapiens
ctgattgtatggaaatataaa

SEQ ID NO: 80 - DNA, 21 bp, Homo sapiens
ctggttgaaacttgtttatta

SEQ ID NO: 81 - DNA, 21 bp, Homo sapiens
ccgccacacggtcacctgcaa

SEQ ID NO: 82 - DNA, 21 bp, Homo sapiens
ccggcgggtctatgactatca

SEQ ID NO: 83 - DNA, 21 bp, Homo sapiens
aaagggagtcattaacaacta

SEQ ID NO: 84 - DNA, 21 bp, Homo sapiens
tacatctacaatggaccagta

SEQ ID NO: 85 - DNA, 21 bp, Homo sapiens
aagatgatgcctagtaaatta

SEQ ID NO: 86 - DNA, 21 bp, Homo sapiens
tggaaagatgatgcctagtaa

SEQ ID NO: 87 - DNA, 21 bp, Homo sapiens
ctcgcgagtgtaactttcata

SEQ ID NO: 88 - DNA, 21 bp, Homo sapiens
acccttcaggtgttactagaa

SEQ ID NO: 89 - DNA, 21 bp, Homo sapiens
cccggagatggagcacgtcta

SEQ ID NO: 90 - DNA, 21 bp, Homo sapiens
gccggtgaccaagattaccaa

SEQ ID NO: 91 - DNA, 21 bp, Homo sapiens
ccagacggcgtccatcttcaa

SEQ ID NO: 92 - DNA, 21 bp, Homo sapiens
cgggaagaagttccgcagcaa

SEQ ID NO: 93 - DNA, 21 bp, Homo sapiens
aagcttctcatcgctactata

SEQ ID NO: 94 - DNA, 21 bp, Homo sapiens
ccgccgaatgacctccgcaaa

SEQ ID NO: 95 - DNA, 21 bp, Homo sapiens
aagagaatctgtgtgtaataa

SEQ ID NO: 96 - DNA, 21 bp, Homo sapiens
ccgaatgacctccgcaaagaa

SEQ ID NO: 97 - DNA, 21 bp, Homo sapiens
cacgatttgacttgctcttca

SEQ ID NO: 98 - DNA, 21 bp, Homo sapiens
cggcaggtatgaccagtataa

SEQ ID NO: 99 - DNA, 21 bp, Homo sapiens
atccaggttgaaggcattcaa

SEQ ID NO: 100 - DNA, 21 bp, Homo sapiens
cagggaatttatcagagcaaa

SEQ ID NO: 101 - DNA, 21 bp, Homo sapiens
cacattgatgtcattcattat

SEQ ID NO: 102 - DNA, 21 bp, Homo sapiens
ctcgacagctagagtcattaa

SEQ ID NO: 103 - DNA, 21 bp, Homo sapiens
ctgcatggccttgatgaagaa

SEQ ID NO: 104 - DNA, 21 bp, Homo sapiens
ccaagcatttatgaacatgaa

SEQ ID NO: 105 - DNA, 21 bp, Homo sapiens
ctggaacaatttaaccagcaa

SEQ ID NO: 106 - DNA, 21 bp, Homo sapiens
tacaatgaagacataaatcaa

SEQ ID NO: 107 - DNA, 21 bp, Homo sapiens
cccagtgaagttggaaccaaa

SEQ ID NO: 108 - DNA, 21 bp, Homo sapiens
tccggttactgaataaatcaa

SEQ ID NO: 109 - DNA, 21 bp, Homo sapiens
ccagaacacgttggaagtgta

SEQ ID NO: 110 - DNA, 21 bp, Homo sapiens
ctggtgctgccagctttataa

SEQ ID NO: 111 - DNA, 21 bp, Homo sapiens
atggctgtcctctctaaggaa

SEQ ID NO: 112 - DNA, 21 bp, Homo sapiens
caagatggctgtcctctctaa

SEQ ID NO: 113 - DNA, 21 bp, Homo sapiens
caaccgaggcatgttcatgaa

SEQ ID NO: 114 - DNA, 21 bp, Homo sapiens
cagggtgtttgtgcctcataa

SEQ ID NO: 115 - DNA, 21 bp, Homo sapiens
ctggcacaggatgaagctgta

SEQ ID NO: 116 - DNA, 21 bp, Homo sapiens
cccgctttgcagatgaagaaa

SEQ ID NO: 117 - DNA, 21 bp, Homo sapiens
cacattaaatatatgagttaa

SEQ ID NO: 118 - DNA, 21 bp, Homo sapiens
ccgccgaaacagacagttaaa

SEQ ID NO: 119 - DNA, 21 bp, Homo sapiens
agggcataaatatatcattca

SEQ ID NO: 120 - DNA, 21 bp, Homo sapiens
gccgccgaaacagacagttaa

SEQ ID NO: 121 - DNA, 21 bp, Homo sapiens
atgcagtgaggtctaacataa

SEQ ID NO: 122 - DNA, 21 bp, Homo sapiens
tcgctcatattaattgatgaa

SEQ ID NO: 123 - DNA, 21 bp, Homo sapiens
atcaattgtcttggatgccaa

SEQ ID NO: 124 - DNA, 21 bp, Homo sapiens
aaccattaacatgagattaga

SEQ ID NO: 125 - DNA, 21 bp, Homo sapiens
ctggagatcctcatacctcaa

SEQ ID NO: 126 - DNA, 21 bp, Homo sapiens
ctgcaaggaatcgatagccaa

SEQ ID NO: 127 - DNA, 21 bp, Homo sapiens
ccgcctcatccttctgcataa

SEQ ID NO: 128 - DNA, 21 bp, Homo sapiens
gagaagatgatcaaacaataa

SEQ ID NO: 129 - DNA, 21 bp, Homo sapiens
aagattaatgttcttcataaa

SEQ ID NO: 130 - DNA, 21 bp, Homo sapiens
caggtaagtataagtattcaa

SEQ ID NO: 131 - DNA, 21 bp, Homo sapiens
ccgcacggctctgaaacttaa

SEQ ID NO: 132 - DNA, 21 bp, Homo sapiens
tacgcgtgccatccaaattaa

SEQ ID NO: 133 - DNA, 21 bp, Homo sapiens
cccaaggacttctaacaataa

SEQ ID NO: 134 - DNA, 21 bp, Homo sapiens
ctcgactaacagggactataa

SEQ ID NO: 135 - DNA, 21 bp, Homo sapiens
gcccaaggacttctaacaata

SEQ ID NO: 136 - DNA, 21 bp, Homo sapiens
cacgtggaaatctccgttatt

SEQ ID NO: 137 - DNA, 21 bp, Homo sapiens
aacgagctgctttaagtgaaa

SEQ ID NO: 138 - DNA, 21 bp, Homo sapiens
cccattggaacaagtaagaaa

SEQ ID NO: 139 - DNA, 21 bp, Homo sapiens
ctgctacttcagtaagaataa

SEQ ID NO: 140 - DNA, 21 bp, Homo sapiens
ctggaaatattgggaagtata

SEQ ID NO: 141 - DNA, 21 bp, Homo sapiens
aacctggtagtgaaacaataa

SEQ ID NO: 142 - DNA, 21 bp, Homo sapiens
aagcaaagcgaagatttccaa

SEQ ID NO: 143 - DNA, 21 bp, Homo sapiens
aaggaggacttgggccaataa

SEQ ID NO: 144 - DNA, 21 bp, Homo sapiens
acagctgtgatcgatggagaa

SEQ ID NO: 145 - DNA, 21 bp, Homo sapiens
aagacctgcaataatccagaa

SEQ ID NO: 146 - DNA, 21 bp, Homo sapiens
cagaatggctactctgatcta

SEQ ID NO: 147 - DNA, 21 bp, Homo sapiens
taaagcattcttcaacagaaa

SEQ ID NO: 148 - DNA, 21 bp, Homo sapiens
tcagatgaatgcggctgttaa

SEQ ID NO: 149 - DNA, 21 bp, Homo sapiens
cagagagaagacagattctta

SEQ ID NO: 150 - DNA, 21 bp, Homo sapiens
cccggcatgggtagcaagaaa

SEQ ID NO: 151 - DNA, 21 bp, Homo sapiens
cacaagtaggatcaagaacaa

SEQ ID NO: 152 - DNA, 21 bp, Homo sapiens
cccagttatcttgacgaatca

SEQ ID NO: 153 - DNA, 21 bp, Homo sapiens
tggataaagaataaagttcta

SEQ ID NO: 154 - DNA, 21 bp, Homo sapiens
ccacatcttcatcaccagcaa

SEQ ID NO: 155 - DNA, 21 bp, Homo sapiens
ctccaccaagtgcatcattaa

SEQ ID NO: 156 - DNA, 21 bp, Homo sapiens
cagcttccagttagtggatat

SEQ ID NO: 157 - DNA, 21 bp, Homo sapiens
accgagcaatataatgagcaa

SEQ ID NO: 158 - DNA, 21 bp, Homo sapiens
ctcaggaaccgtggaccttaa

SEQ ID NO: 159 - DNA, 21 bp, Homo sapiens
tacgccttacaccatgagcta

SEQ ID NO: 160 - DNA, 21 bp, Homo sapiens
cagacttgaccgagcaatata

SEQ ID NO: 161 - DNA, 21 bp, Homo sapiens
cagcttgaaactattaacgta

SEQ ID NO: 162 - DNA, 21 bp, Homo sapiens
ttggagatatattcagaataa

SEQ ID NO: 163 - DNA, 21 bp, Homo sapiens
catgctgaaatggtaaataaa

SEQ ID NO: 164 - DNA, 21 bp, Homo sapiens
aagaaagaacataacagagta

SEQ ID NO: 165 - DNA, 21 bp, Homo sapiens
ctggtgggtctctagaagcaa

SEQ ID NO: 166 - DNA, 21 bp, Homo sapiens
aacggatatattgagaatcaa

SEQ ID NO: 167 - DNA, 21 bp, Homo sapiens
ctgagagtatataacaacaca

SEQ ID NO: 168 - DNA, 21 bp, Homo sapiens
ccgagatttctcttacaatta

SEQ ID NO: 169 - DNA, 21 bp, Homo sapiens
ccggtagacttaggtctgtta

SEQ ID NO: 170 - DNA, 21 bp, Homo sapiens
aagaagtgttgaagtaaccta

SEQ ID NO: 171 - DNA, 21 bp, Homo sapiens
aacccaggatgggacactaaa

SEQ ID NO: 172 - DNA, 21 bp, Homo sapiens
ctgcttggatttatttgtgtt

SEQ ID NO: 173 - DNA, 21 bp, Homo sapiens
cagagtgtacttacctaacaa

SEQ ID NO: 174 - DNA, 21 bp, Homo sapiens
tacatatattatctacatcaa

SEQ ID NO: 175 - DNA, 21 bp, Homo sapiens
acaactaatgttaacgcataa

SEQ ID NO: 176 - DNA, 21 bp, Homo sapiens
taccttctacgatacaactaa

SEQ ID NO: 177 - DNA, 21 bp, Homo sapiens
aacgatttaaaggtacaacaa

SEQ ID NO: 178 - DNA, 21 bp, Homo sapiens
ctcgagatgatagtcaactaa

SEQ ID NO: 179 - DNA, 21 bp, Homo sapiens
accggataacacggccttcaa

SEQ ID NO: 180 - DNA, 21 bp, Homo sapiens
atcgatggcgatgaactataa

SEQ ID NO: 181 - DNA, 21 bp, Homo sapiens
aacaccctctatgaaatcaaa

SEQ ID NO: 182 - DNA, 21 bp, Homo sapiens
aagcgtgtttctgcaatagta

SEQ ID NO: 183 - DNA, 21 bp, Homo sapiens
ccctaagtgaataaactcaat

SEQ ID NO: 184 - DNA, 21 bp, Homo sapiens
atggaacatttaaacttacaa

SEQ ID NO: 185 - DNA, 21 bp, Homo sapiens
cccgatggcatcaaggtcttt

SEQ ID NO: 186 - DNA, 21 bp, Homo sapiens
tccctccaactctgtctctaa

SEQ ID NO: 187 - DNA, 21 bp, Homo sapiens
ccggccggccgcagccatgaa

SEQ ID NO: 188 - DNA, 21 bp, Homo sapiens
ccgcctgctcttggagaacta

SEQ ID NO: 189 - DNA, 21 bp, Homo sapiens
cacgtacaacacgcaggtcaa

SEQ ID NO: 190 - DNA, 21 bp, Homo sapiens
ccgcgtgtacaagtcactgaa

SEQ ID NO: 191 - DNA, 21 bp, Homo sapiens
cagctacgtttgtacatcaaa

SEQ ID NO: 192 - DNA, 21 bp, Homo sapiens
ccggaccttgtcttcgaggaa

SEQ ID NO: 193 - DNA, 21 bp, Homo sapiens
cagggtactacgcaagcctta

SEQ ID NO: 194 - DNA, 21 bp, Homo sapiens
ttgcaacgacacaaactataa

SEQ ID NO: 195 - DNA, 21 bp, Homo sapiens
agggtactacgcaagccttaa

SEQ ID NO: 196 - DNA, 21 bp, Homo sapiens
cacgatgtatacttcccaaat

SEQ ID NO: 197 - DNA, 21 bp, Homo sapiens
aacactctaagcataactaaa

SEQ ID NO: 198 - DNA, 21 bp, Homo sapiens
acgcacggacatcaccatgaa

SEQ ID NO: 199 - DNA, 21 bp, Homo sapiens
ccagtggagataacactctaa

SEQ ID NO: 200 - DNA, 21 bp, Homo sapiens
ctgggcgaatgtcttatttaa

SEQ ID NO: 201 - DNA, 21 bp, Homo sapiens
aagggcaatggagataaatta

SEQ ID NO: 202 - DNA, 21 bp, Homo sapiens
cagtgaaggtatggaatatta

SEQ ID NO: 203 - DNA, 21 bp, Homo sapiens
ccgcattctgatgcttcgcaa

SEQ ID NO: 204 - DNA, 21 bp, Homo sapiens
taggcagagtataaagtatta

SEQ ID NO: 205 - DNA, 21 bp, Homo sapiens
atcatcagatgtggtctataa

SEQ ID NO: 206 - DNA, 21 bp, Homo sapiens
cagctctgagcagatcatgaa

SEQ ID NO: 207 - DNA, 21 bp, Homo sapiens
cagggtttcatccaggatcga

SEQ ID NO: 208 - DNA, 21 bp, Homo sapiens
ccgagtggcagctgacatgtt

SEQ ID NO: 209 - DNA, 21 bp, Homo sapiens
cagcctgacaaggtccgcaaa

SEQ ID NO: 210 - DNA, 21 bp, Homo sapiens
gcccgctcatttgttaataaa

SEQ ID NO: 211 - DNA, 21 bp, Homo sapiens
aaggctgagtaccatgggcta

SEQ ID NO: 212 - DNA, 21 bp, Homo sapiens
caccctgagtcttgcgtccaa

SEQ ID NO: 213 - DNA, 21 bp, Homo sapiens
aagaatcgcgtggctcatcaa

SEQ ID NO: 214 - DNA, 21 bp, Homo sapiens
ttcgtgataaaccacatgcta

SEQ ID NO: 215 - DNA, 21 bp, Homo sapiens
tcagcagaatctacaaatata

SEQ ID NO: 216 - DNA, 21 bp, Homo sapiens
acgtggctggacaaacatcta

SEQ ID NO: 217 - DNA, 21 bp, Homo sapiens
cactgtagatgctataataaa

SEQ ID NO: 218 - DNA, 21 bp, Homo sapiens
tagcgtccttgttgacataaa

SEQ ID NO: 219 - DNA, 21 bp, Homo sapiens
ccacttctgtgattaagtaaa

SEQ ID NO: 220 - DNA, 21 bp, Homo sapiens
ccgcgataatcgtgtggtgta

SEQ ID NO: 221 - DNA, 21 bp, Homo sapiens
atgattcttagtgactttaaa

SEQ ID NO: 222 - DNA, 21 bp, Homo sapiens
cagtttgtgtgtcttaattat

SEQ ID NO: 223 - DNA, 21 bp, Homo sapiens
ctggcattagaattatttaaa

SEQ ID NO: 224 - DNA, 21 bp, Homo sapiens
ctctggcattagaattattta

SEQ ID NO: 225 - DNA, 21 bp, Homo sapiens
cacaatcaagatctgtatcaa

SEQ ID NO: 226 - DNA, 21 bp, Homo sapiens
tcgggacaaattagctgcaca

SEQ ID NO: 227 - DNA, 21 bp, Homo sapiens
caccagtgttatcaacttgaa

SEQ ID NO: 228 - DNA, 21 bp, Homo sapiens
ctagcataatttgtattgaaa

SEQ ID NO: 229 - DNA, 21 bp, Homo sapiens
cgggtgctggatgacaaggaa

SEQ ID NO: 230 - DNA, 21 bp, Homo sapiens
gagagtcaccttcgacttgta

SEQ ID NO: 231 - DNA, 21 bp, Homo sapiens
acgcatttcatgatcttggaa

SEQ ID NO: 232 - DNA, 21 bp, Homo sapiens
cacgcatttcatgatcttgga

SEQ ID NO: 233 - DNA, 21 bp, Homo sapiens
gagccttgtgctgtcaataaa

SEQ ID NO: 234 - DNA, 21 bp, Homo sapiens
ctggcacaagttctgcctcaa

SEQ ID NO: 235 - DNA, 21 bp, Homo sapiens
cccacctgccagtgttattta

SEQ ID NO: 236 - DNA, 21 bp, Homo sapiens
acggtttgaggattgcagaaa

SEQ ID NO: 237 - DNA, 21 bp, Homo sapiens
aacgtagttatcagcgattca

SEQ ID NO: 238 - DNA, 21 bp, Homo sapiens
accgacagcactcaaattaaa

SEQ ID NO: 239 - DNA, 21 bp, Homo sapiens
ctcaggattgatacatttcaa

SEQ ID NO: 240 - DNA, 21 bp, Homo sapiens
cgggttcgagtacacctctaa

SEQ ID NO: 241 - DNA, 21 bp, Homo sapiens
cagcctaacttacattcttaa

SEQ ID NO: 242 - DNA, 21 bp, Homo sapiens
ccagcctaacttacattctta

SEQ ID NO: 243 - DNA, 21 bp, Homo sapiens
cgccgatggcactgccattaa

SEQ ID NO: 244 - DNA, 21 bp, Homo sapiens
cccacaggagaagattgtcaa

SEQ ID NO: 245 - DNA, 21 bp, Homo sapiens
cccatcggtttccgcgcgaaa

SEQ ID NO: 246 - DNA, 21 bp, Homo sapiens
tcgcttatcaactaatgattt

SEQ ID NO: 247 - DNA, 21 bp, Homo sapiens
tcccgagtatgcgcccatatt

SEQ ID NO: 248 - DNA, 21 bp, Homo sapiens
cccaatgagactgacatcaaa

SEQ ID NO: 249 - DNA, 21 bp, Homo sapiens
cagcacctcgatgtttataaa

SEQ ID NO: 250 - DNA, 21 bp, Homo sapiens
ctcgccgtgtaacaaatgaaa

SEQ ID NO: 251 - DNA, 21 bp, Homo sapiens
agcaatgacattagttccaaa

SEQ ID NO: 252 - DNA, 21 bp, Homo sapiens
cgcaagagtatcagtgattta

SEQ ID NO: 253 - DNA, 21 bp, Homo sapiens
aacgccgaggagagcttgtaa

SEQ ID NO: 254 - DNA, 21 bp, Homo sapiens
tagcctgaggataaaggctga

SEQ ID NO: 255 - DNA, 21 bp, Homo sapiens
ctgacttgaataatttatcaa

SEQ ID NO: 256 - DNA, 21 bp, Homo sapiens
tcgaatctggatgatgctaaa

SEQ ID NO: 257 - DNA, 21 bp, Homo sapiens
aagcaggaatggcttgcttaa

SEQ ID NO: 258 - DNA, 21 bp, Homo sapiens
ctgattaaaggtcagcactta

SEQ ID NO: 259 - DNA, 21 bp, Homo sapiens
ctgaggccttgtgtcctttaa

SEQ ID NO: 260 - DNA, 21 bp, Homo sapiens
cccgtgggtgctggctgccaa

SEQ ID NO: 261 - DNA, 21 bp, Homo sapiens
cgcgctgaaatttggcgtgaa

SEQ ID NO: 262 - DNA, 21 bp, Homo sapiens
acggtgcccatcaatctcata

SEQ ID NO: 263 - DNA, 21 bp, Homo sapiens
ctgaaatttggcgtgaattaa

SEQ ID NO: 264 - DNA, 21 bp, Homo sapiens
taccatcagctccatcaacaa

SEQ ID NO: 265 - DNA, 21 bp, Homo sapiens
atggaccatttaggaattata

SEQ ID NO: 266 - DNA, 21 bp, Homo sapiens
caggagcgagtgggtgaatta

SEQ ID NO: 267 - DNA, 21 bp, Homo sapiens
cacgtctttgtaatagcagaa

SEQ ID NO: 268 - DNA, 21 bp, Homo sapiens
ttcccttacccagaaatgaaa

SEQ ID NO: 269 - DNA, 21 bp, Homo sapiens
acgaattggcatggtggtgaa

SEQ ID NO: 270 - DNA, 21 bp, Homo sapiens
ccgggctggtttgattccgaa

SEQ ID NO: 271 - DNA, 21 bp, Homo sapiens
accagtggtggaacagttgaa

SEQ ID NO: 272 - DNA, 21 bp, Homo sapiens
cagattcaagaacaagggaaa

SEQ ID NO: 273 - DNA, 21 bp, Homo sapiens
caggaccactattaagccata

SEQ ID NO: 274 - DNA, 21 bp, Homo sapiens
ttccatcaggaccactattaa

SEQ ID NO: 275 - DNA, 21 bp, Homo sapiens
ctgctgggacaagacactgta

SEQ ID NO: 276 - DNA, 21 bp, Homo sapiens
cacgtttacccgcaagccgaa

SEQ ID NO: 277 - DNA, 21 bp, Homo sapiens
aagaaagatattgtttcttta

SEQ ID NO: 278 - DNA, 21 bp, Homo sapiens
caccttcatgatcgacctcaa

SEQ ID NO: 279 - DNA, 21 bp, Homo sapiens
taggaagactcccggattcta

SEQ ID NO: 280 - DNA, 21 bp, Homo sapiens
ttgccagacattagtggataa

SEQ ID NO: 281 - DNA, 21 bp, Homo sapiens
ctgggtatacttcatgtgaca

SEQ ID NO: 282 - DNA, 21 bp, Homo sapiens
gacgccatctatgacagtaaa

SEQ ID NO: 283 - DNA, 21 bp, Homo sapiens
accgtgtaaaccaaagcccta

SEQ ID NO: 284 - DNA, 21 bp, Homo sapiens
cgcggtgacaggagacgtgaa

SEQ ID NO: 285 - DNA, 21 bp, Homo sapiens
aagatttatgatgcacttatt

SEQ ID NO: 286 - DNA, 21 bp, Homo sapiens
caatttgagattctacagtaa

SEQ ID NO: 287 - DNA, 21 bp, Homo sapiens
acgggaagacaagttcatgta

SEQ ID NO: 288 - DNA, 21 bp, Homo sapiens
tcggcttctcctgaaagggaa

SEQ ID NO: 289 - DNA, 21 bp, Homo sapiens
cccgtggacttcaagaagata

SEQ ID NO: 290 - DNA, 21 bp, Homo sapiens
ccgcgctacaaccagatgaaa

SEQ ID NO: 291 - DNA, 21 bp, Homo sapiens
tcactggatgtcaaacagtaa

SEQ ID NO: 292 - DNA, 21 bp, Homo sapiens
ccgcagtttggagtcactgta

SEQ ID NO: 293 - DNA, 21 bp, Homo sapiens
atccaccgtgttgcagatata

SEQ ID NO: 294 - DNA, 21 bp, Homo sapiens
caggctgaacctgtggcgcta

SEQ ID NO: 295 - DNA, 21 bp, Homo sapiens
acggtggactacattagacca

SEQ ID NO: 296 - DNA, 21 bp, Homo sapiens
tcgaaagaagctgattgggaa

SEQ ID NO: 297 - DNA, 21 bp, Homo sapiens
aaggacagacgaagagtttaa

SEQ ID NO: 298 - DNA, 21 bp, Homo sapiens
cacggtcaaactgaaatggat

SEQ ID NO: 299 - DNA, 21 bp, Homo sapiens
tcctttctacttgtcgctaaa

SEQ ID NO: 300 - DNA, 21 bp, Homo sapiens
ccgcgagaaacttgcattgga

SEQ ID NO: 301 - DNA, 21 bp, Homo sapiens
cagttacctaatcatgttgaa

SEQ ID NO: 302 - DNA, 21 bp, Homo sapiens
ctgaatcaaggatttaattaa

SEQ ID NO: 303 - DNA, 21 bp, Homo sapiens
ctgaagtgccttctgaatcaa

SEQ ID NO: 304 - DNA, 21 bp, Homo sapiens
caggttgaagtcaagatgaca

SEQ ID NO: 305 - DNA, 21 bp, Homo sapiens
caccgttattgctaaagtaaa

SEQ ID NO: 306 - DNA, 21 bp, Homo sapiens
gaggaacagcgtttccgtaaa

SEQ ID NO: 307 - DNA, 21 bp, Homo sapiens
acaccgttattgctaaagtaa

SEQ ID NO: 308 - DNA, 21 bp, Homo sapiens
aagccgattggtgctggtgaa

SEQ ID NO: 309 - DNA, 21 bp, Homo sapiens
atcaaacttcatgatcaatca

SEQ ID NO: 310 - DNA, 21 bp, Homo sapiens
ccgggtccacaagaccttcaa

SEQ ID NO: 311 - DNA, 21 bp, Homo sapiens
caggcaatggacataatatat

SEQ ID NO: 312 - DNA, 21 bp, Homo sapiens
caggaactttccggaatgaca

SEQ ID NO: 313 - DNA, 21 bp, Homo sapiens
aaggactctctcacacaggaa

SEQ ID NO: 314 - DNA, 21 bp, Homo sapiens
acccatatcaagggtgactaa

SEQ ID NO: 315 - DNA, 21 bp, Homo sapiens
tagggtgtggtcactctcaat

SEQ ID NO: 316 - DNA, 21 bp, Homo sapiens
cactaggaggctgatcttgta

SEQ ID NO: 317 - DNA, 21 bp, Homo sapiens
cagcatcttatccgagtggaa

SEQ ID NO: 318 - DNA, 21 bp, Homo sapiens
ttgcagttaagggttagttta

SEQ ID NO: 319 - DNA, 21 bp, Homo sapiens
ttggtcgaccttagtacctaa

SEQ ID NO: 320 - DNA, 21 bp, Homo sapiens
cagagtgcattgtgagggtta

SEQ ID NO: 321 - DNA, 21 bp, Homo sapiens
ccggctggtattgtctctaca

SEQ ID NO: 322 - DNA, 21 bp, Homo sapiens
tagcaaatggcttctatcgaa

SEQ ID NO: 323 - DNA, 21 bp, Homo sapiens
tcggagggcgatgaaacgttt

SEQ ID NO: 324 - DNA, 21 bp, Homo sapiens
ccagtggagatagagattgaa

SEQ ID NO: 325 - DNA, 21 bp, Homo sapiens
cagaattattgctgcaattaa

SEQ ID NO: 326 - DNA, 21 bp, Homo sapiens
gagacacttaagggaaattaa

SEQ ID NO: 327 - DNA, 21 bp, Homo sapiens
ccgctgtgaatttgacagcca

SEQ ID NO: 328 - DNA, 21 bp, Homo sapiens
aagccaaactgaaatcggtaa

SEQ ID NO: 329 - RNA, 19 nt, Artificial Sequence
  siRNA
gaaccaauuuaucacccau

SEQ ID NO: 330 - RNA, 19 nt, Artificial Sequence
  siRNA
ccacaugugccgagaauuc

SEQ ID NO: 331 - RNA, 19 nt, Artificial Sequence
  siRNA
gcacugaagccgagcucaa

SEQ ID NO: 332 - RNA, 19 nt, Artificial Sequence
  siRNA
auacuucccugacuuggua

SEQ ID NO: 333 - RNA, 19 nt, Artificial Sequence
  siRNA
gcgaugaacuauaacgcga

SEQ ID NO: 334 - RNA, 19 nt, Artificial Sequence
  siRNA
ccaucgucguuacgugaaa

SEQ ID NO: 335 - RNA, 19 nt, Artificial Sequence
  siRNA
gcacagaggaugucgcuaa

SEQ ID NO: 336 - RNA, 19 nt, Artificial Sequence
  siRNA
gcgagaucgagauugauua

SEQ ID NO: 337 - RNA, 19 nt, Artificial Sequence
  siRNA
cugauugccuguaauguua

SEQ ID NO: 338 - RNA, 19 nt, Artificial Sequence
  siRNA
gcagguaugaccaguauaa

SEQ ID NO: 339 - RNA, 19 nt, Artificial Sequence
  siRNA
gaccauagagcgacguuau

SEQ ID NO: 340 - RNA, 19 nt, Artificial Sequence
  siRNA
cuaaccggcuucugaacaa

SEQ ID NO: 341 - RNA, 19 nt, Artificial Sequence
  siRNA
guuauaagauggagacuuc

SEQ ID NO: 342 - RNA, 19 nt, Artificial Sequence
  siRNA
agacugggcuggcuuagau

SEQ ID NO: 343 - RNA, 19 nt, Artificial Sequence
  siRNA
guuacgagcugaacuagag

SEQ ID NO: 344 - RNA, 19 nt, Artificial Sequence
  siRNA
caauguagaggaugacuuc

SEQ ID NO: 345 - RNA, 19 nt, Artificial Sequence
  siRNA
gaagauuugauguguacuu

SEQ ID NO: 346 - RNA, 19 nt, Artificial Sequence
  siRNA
ggaacagaaugccguaagu

SEQ ID NO: 347 - RNA, 19 nt, Artificial Sequence
  siRNA
gaagauaccaucccacgaa

SEQ ID NO: 348 - RNA, 19 nt, Artificial Sequence
  siRNA
uaacuuuacuuccacucau

SEQ ID NO: 349 - RNA, 19 nt, Artificial Sequence
  siRNA
gggcuacgcugauucgaga

SEQ ID NO: 350 - RNA, 19 nt, Artificial Sequence
  siRNA
gagggagcuagcaacguua

SEQ ID NO: 351 - RNA, 19 nt, Artificial Sequence
  siRNA
ccuucagauucuucguaaa

SEQ ID NO: 352 - RNA, 19 nt, Artificial Sequence
  siRNA
agaaagaguccacggguac

SEQ ID NO: 353 - RNA, 19 nt, Artificial Sequence
  siRNA
uaacacaggccgagcgaau

SEQ ID NO: 354 - RNA, 19 nt, Artificial Sequence
  siRNA
gcuacgacauccugguuaa

SEQ ID NO: 355 - RNA, 19 nt, Artificial Sequence
  siRNA
agaaauaugggaaugauca

SEQ ID NO: 356 - RNA, 19 nt, Artificial Sequence
  siRNA
ggacaucacgguuucucau

SEQ ID NO: 357 - RNA, 19 nt, Artificial Sequence
  siRNA
gcugugacauagauauuua

SEQ ID NO: 358 - RNA, 19 nt, Artificial Sequence
  siRNA
ugggagaucucaaguuuca

SEQ ID NO: 359 - RNA, 19 nt, Artificial Sequence
  siRNA
gggaauccaaucuguugca

SEQ ID NO: 360 - RNA, 19 nt, Artificial Sequence
  siRNA
guugaauugccaccuguuu

SEQ ID NO: 361 - RNA, 19 nt, Artificial Sequence
  siRNA
uaaauaaccuccuugauga

SEQ ID NO: 362 - RNA, 19 nt, Artificial Sequence
  siRNA
cagaagucauuaauugcua

SEQ ID NO: 363 - RNA, 19 nt, Artificial Sequence
  siRNA
cggaaagagugcaucugua

SEQ ID NO: 364 - RNA, 19 nt, Artificial Sequence
  siRNA
gcuauuggccgacauaugu

SEQ ID NO: 365 - RNA, 19 nt, Artificial Sequence
  siRNA
agucaagagucaucgaaua

SEQ ID NO: 366 - RNA, 19 nt, Artificial Sequence
  siRNA
gauaguauggucaacaaua

SEQ ID NO: 367 - RNA, 19 nt, Artificial Sequence
  siRNA
uaacccaagucgcuauaua

SEQ ID NO: 368 - RNA, 19 nt, Artificial Sequence
  siRNA
gugcaaagccugggauaua

SEQ ID NO: 369 - RNA, 19 nt, Artificial Sequence
  siRNA
ggacuaugguguauaccua

SEQ ID NO: 370 - RNA, 19 nt, Artificial Sequence
  siRNA
cgugggaaauacuugguuu

SEQ ID NO: 371 - RNA, 19 nt, Artificial Sequence
  siRNA
ggauuccacuucuuucaga

SEQ ID NO: 372 - RNA, 19 nt, Artificial Sequence
  siRNA
gaugagacacuacguuugg

SEQ ID NO: 373 - RNA, 19 nt, Artificial Sequence
  siRNA
cgacugugaugcgcuaaug

SEQ ID NO: 374 - RNA, 19 nt, Artificial Sequence
  siRNA
ccuaauccgcccacaggaa

SEQ ID NO: 375 - RNA, 19 nt, Artificial Sequence
  siRNA
cgucagaacccaugcggca

SEQ ID NO: 376 - RNA, 19 nt, Artificial Sequence
  siRNA
agaccagcaugacagauuu

SEQ ID NO: 377 - RNA, 19 nt, Artificial Sequence
  siRNA
gaaauuugcguguggagua

SEQ ID NO: 378 - RNA, 19 nt, Artificial Sequence
  siRNA
gugcagcuguggguugauu

SEQ ID NO: 379 - RNA, 19 nt, Artificial Sequence
  siRNA
gcagucagauccuagcguc

SEQ ID NO: 380 - RNA, 19 nt, Artificial Sequence
  siRNA
ggagaauauuucacccuuc

(19) United States
(12) Patent Application Publication
(10) Pub. No.: US 2012/0046186 A1
(43) Pub. Date: Feb. 23, 2012

(54) Gene Expression Markers for Prediction of Response to Platinum-Based Chemotherapy Drugs

(76) Inventors: ROBERT J. PELHAM, BELMONT, CA (US); KELLY A. HARRADINE, PACIFICA, CA (US); JOFFRE B. BAKER, MONTARA, CA (US)

(21) Appl. No.: 13/211,148

(22) Filed: Aug. 16, 2011

Related U.S. Application Data
(60) Provisional application No. 61/375,782, filed on Aug. 20, 2010.

(51) Int. Cl. C40B 30/04; G01N 33/566; G01N 33/577; C12Q 1/68

(52) U.S. Cl. 506/9; 435/6.11; 435/7.1; 435/7.9

(57) ABSTRACT

The present invention provides methods for predicting a likelihood that a patient with cancer will exhibit a positive response to a treatment with a platinum-based chemotherapy drug. The methods generally involve determining an expression level of a gene product that correlates with responsiveness to treatment with a platinum-based chemotherapy drug. In an embodiment of the invention, the platinum-based chemotherapy drug is oxaliplatin, and the cancer is colorectal cancer.

This application claims priority under 35 U.S.C. §119(e) to U.S. Provisional Application No. 61/375,782, filed on Aug. 20, 2010, which is hereby incorporated by reference in its entirety.

### FIELD OF THE INVENTION

The present invention relates to genes, the expression levels of which are useful for predicting response of cancer cells and cancer patients to a platinum-based chemotherapy drug.

### BACKGROUND

Platinum-based cancer chemotherapies have had a major clinical impact in the treatment of patients with cancer. Furthermore, an emerging clinical strategy is that the optimal efficacy of novel targeted therapies may be in combination with existing cytotoxic DNA-damaging agents, including oxaliplatin. Given the expanding role of oxaliplatin in cancer treatment, it has become increasingly important to understand molecular predictors of oxaliplatin response in order to provide for more personalized administration of chemotherapy.

Oxaliplatin is a third-generation platinum-based chemotherapeutic agent that has significant activity in colorectal cancer (CRC). Adjuvant therapy with oxaliplatin, combined with fluoropyrimidine-based chemotherapy, results in significant increases in disease-free survival rates in patients with stage II/III colon cancer (Andre, T., et al., “Oxaliplatin, Fluorouracil, and Leucovorin as Adjuvant Treatment for Colon Cancer,” N. Engl. J. Med., 2004. 350(23): p. 2343-51). In the metastatic setting, combination therapy with 5-FU and oxaliplatin is the most commonly used front-line regimen, with superior response rates and longer survival than 5-FU alone (Rothenberg, M. L., et al., “Superiority of Oxaliplatin and Fluorouracil-Leucovorin Compared with Either Therapy Alone in Patients with Progressive Colorectal Cancer After Irinotecan and Fluorouracil-Leucovorin: Interim Results of a Phase III Trial,” J. Clin. Oncol., 2003. 21(11): p. 2059-69; de Gramont, A., et al., “Reintroduction of Oxaliplatin is Associated With Improved Survival in Advanced Colorectal Cancer,” J. Clin. Oncol., 2007. 25(22): p. 3224-9). However, it is apparent that not all patients benefit from oxaliplatin treatment, and in the face of significant side-effects associated with oxaliplatin, most notably prolonged neurotoxicity, there is a need for clinical tools to guide use of oxaliplatin in those patients who are most likely to derive benefit.

Oxaliplatin induces cytotoxicity through the formation of platinum-DNA adducts, which in turn, activate multiple signaling pathway (Kelland, L., “The Resurgence of Platinum-Based Cancer Chemotherapy,” Nat. Rev. Cancer, 2007. 7(8): p. 573-84). Alterations in drug efflux and uptake, DNA repair and inactivation of the apoptosis pathways have been hypothesized to promote resistance to platinum agents such as carboplatin and cisplatin (Wang, D. and S. J. Lippard, “Cellular Processing of Platinum Anticancer Drugs,” Nat. Rev. Drug Discov., 2005. 4(4): p. 307-320; Siddick, Z. H., “Cisplatin: Mode of Cytotoxic Action and Molecular Basis of Resistance,” Oncogene, 2003. 22(47): p. 7265-79). None of these putative markers of oxaliplatin sensitivity and resistance have been clinically validated, and at present, there are no markers established in clinical use for selecting CRC patients for oxaliplatin therapy.

The current clinical practice used for making CRC treatment decisions is determined by clinical and pathological staging. However, these prognostic tools do not predict drug response in an individual patient. Recent insights into the genomics of cancers have enabled development of diagnostic tests that inform clinical decisions for cancer patients (Harris, L., et al., “American Society of Clinical Oncology 2007 Update of Recommendations for the Use of Tumor Markers in Breast Cancer,” J. Clin. Oncol., 2007. 25(33): p. 5287-312; Dunn., L. and A. Demichele, “Genomic Predictors of Outcome and Treatment Response in Breast Cancer,” Mol. Diagn. Ther., 2009. 13(2): p. 73-90; Paik, S., et al., “A Multigene Assay to Predict Recurrence of Tamoxifen-Treated, Node-Negative Breast Cancer,” N. Engl. J. Med., 2004. 351(27): p. 2817-25; Paik, S., et al., “Gene Expression and Benefit of Chemotherapy in Women With Node-Negative, Estrogen Receptor-Positive Breast Cancer,” J. Clin. Oncol., 2006. 24(23): p. 3726-34). To further advance the personalization of CRC treatment, there is a need for a greater understanding of the genetic alterations in CRC tumors that are associated with patient sensitivity or resistance to oxaliplatin.

### SUMMARY

The present invention provides response indicator genes for platinum-based chemotherapy drugs. These genes are provided in Tables 1-4. The present invention also provides gene subsets of the response indicator genes based on their known function. These gene subsets include, but are not limited to, a drug resistance group, drug transporter group, apoptosis group, DNA damage repair group, cell cycle group, p53 pathway group, and nucleotide excision repair (NER) group. Table 1 provides a gene subset in which each gene may be grouped. The present invention also provides methods of identifying gene cliques, i.e. genes that co-express with a response indicator gene and exhibit correlation of expression with the response indicator gene, and thus may be substituted for that response indicator gene in an assay.

In an embodiment of the invention, increased expression level of one or more response indicator genes selected from ATP6V0C, BCL10, BCL2L10, BFAR, BRIP1, CARD6, CCND1, CDC20, CDC25A, CFLAR, CHAF1A, CRADD, CUL4B, DFFA, E2F2, E2F4, E2F6, GADD45B, HMG20B, IL8, LTBR, MBD2, MBD3, MBD4, MCM3, MCM4, MCM6, MGST3, MPG, MRPL3, MSH4, NHEJ1, OGT, PAICS, PPP2R5C, PRDX4, PTTG1, RAD51L1, RARA, RBM4, RECQL, RRM1, SHFM1, SPO11, TMEM30A, UBE2A, UBE2S, XAB2, and XRCC2 is negatively correlated with a likelihood of a positive response to a platinum-based chemotherapy drug.

In another embodiment of the invention, increased expression level of one or more response indicator genes selected from ABL1, APAF1, BAX, CARD4, CASP5, CCT5, CDKN1A, CDKN3, CIDEA, CRIP2, CUL1, CYP1A2, DNMT1, ERCC4, FANCE, GSTT1, GSTZ1, GTF2H5, KPNA2, MRPS12, MSH5, NFKB1, PTEN, SMARCA4, SND1, SOX4, SUMO1, TARS, TNFRSF10A, TNFSF8, TP53, XPC, and XRCC3 is positively correlated with a likelihood of a positive response to a platinum-based chemotherapy drug.

In a specific embodiment of the invention, increased expression level of one or more genes selected from BCL10, BCL2L10, BFAR, BRIP1, CHAF1A, CUL4B, DFFA, IL8, LTBR, MBD2, MBD4, MCM3, MCM4, MCM6, MPG, MSH4, NHEJ1, PRDX4, PTTG1, RAD51L1, RRM1, SHFM1, and TMEM30A is negatively correlated with a likelihood of a positive response to a platinum-based chemotherapy drug, and increased expression level of one or more genes selected from CDKN1A, KPNA2, SUMO1, and TP53 is positively correlated with a likelihood of a positive response to a platinum-based chemotherapy drug.

The present invention further provides methods and compositions for predicting the likelihood that a patient with cancer will exhibit a positive response to a treatment comprising a platinum-based chemotherapy drug based on the expression level of one or more response indicator genes in a tumor sample obtained from the patient. Specifically, the method comprises assaying or measuring an expression level of one or more response indicator gene products. The response indicator gene is selected from any one of the genes listed in Tables 1-4. In an embodiment of the invention, the response indicator gene is one or more selected from ABL1, APAF1, ATP6V0C, BAX, BCL10, BCL2L10, BFAR, BRIP1, CARD4, CARD6, CASP5, CCND1, CCT5, CDC20, CDC25A, CDKN1A, CDKN3, CFLAR, CHAF1A, CIDEA, CRADD, CRIP2, CUL1, CUL4B, CYP1A2, DFFA, DNMT1, E2F2, E2F4, E2F6, ERCC4, FANCE, GADD45B, GSTT1, GSTZ1, GTF2H5, HMG20B, IL8, KPNA2, LTBR, MBD2, MBD3, MBD4, MCM3, MCM4, MCM6, MGST3, MPG, MRPL3, MRPS12, MSH4, MSH5, NFKB1, NHEJ1, OGT, PAICS, PPP2R5c, PRDX4, PTEN, PTTG1, RAD51L1, RARA, RBM4, RECQL, RRM1, SHFM1, SMARCA4, SND1, SOX4, SPO11, SUMO1, TARS, TMEM30A, TNFRSF10A, TNFSF8, TP53, UBE2A, UBE2S, XAB2, XPC, XRCC2, and XRCC3. In another embodiment of the invention, the response indicator gene is one or more selected from BCL10, BCL2L10, BFAR, BRIP1, CDKN1A, CHAF1A, CUL4B, DFFA, IL8, KPNA2, LTBR, MBD2, MBD4, MCM3, MCM4, MCM6, MPG, MSH4, NHEJ1, PRDX4, PTTG1, RAD51L1, RRM1, SHFM1, SUMO1, TMEM30A, and TP53. In a further embodiment, the expression level of the response indicator gene is normalized. The expression level or the normalized expression level is used to predict the likelihood of a positive response, wherein increased expression level or increased normalized expression level of one or more response indicator genes selected from ATP6V0C, BCL10, BCL2L10, BFAR, BRIP1, CARD6, CCND1, CDC20, CDC25A, CFLAR, CHAF1A, CRADD, CUL4B, DFFA, E2F2, E2F4, E2F6, GADD45B, HMG20B, IL8, LTBR, MBD2, MBD3, MBD4, MCM3, MCM4, MCM6, MGST3, MPG, MRPL3, MSH4, NHEJ1, OGT, PAICS, PPP2R5c, PRDX4, PTTG1, RAD51L1, RARA, RBM4, RECQL, RRM1, SHFM1, SPO11, TMEM30A, UBE2A, UBE2S, XAB2, and XRCC2 is negatively correlated with a likelihood that the patient will exhibit a positive response to a treatment comprising a platinum-based chemotherapy drug, and increased expression level or increased normalized expression level of one or more response indicator genes selected from ABL1, APAF1, BAX, CARD4, CASP5, CCT5, CDKN1A, CDKN3, CIDEA, CRIP2, CUL1, CYP1A2, DNMT1, ERCC4, FANCE, GSTT1, GSTZ1, GTF2H5, KPNA2, MRPS12, MSH5, NFKB1, PTEN, SMARCA4, SND1, SOX4, SUMO1, TARS, TNFRSF10A, TNFSF8, TP53, XPC, and XRCC3 is positively correlated with a likelihood that the patient will exhibit a positive response to a treatment comprising a platinum-based chemotherapy drug. In yet another embodiment of the invention, increased expression level of one or more genes selected from BCL10, BCL2L10, BFAR, BRIP1, CHAF1A, CUL4B, DFFA, IL8, LTBR, MBD2, MBD4, MCM3, MCM4, MCM6, MPG, MSH4, NHEJ1, PRDX4, PTTG1, RAD51L1, RRM1, SHFM1, and TMEM30A is negatively correlated with a likelihood that the patient will exhibit a positive response to a treatment comprising a platinum-based chemotherapy drug, and increased expression level of one or more genes selected from CDKN1A, KPNA2, SUMO1, and TP53 is positively correlated with a likelihood that the patient will exhibit a positive response to treatment comprising a platinum-based chemotherapy drug. In a further embodiment of the invention, a report is generated based on the predicted likelihood of response.

The methods of the present invention contemplate determining the expression level of at least one response indicator gene or its gene product. For all aspects of the present invention, the methods may further include determining the expression levels of at least two response indicator genes, or their expression products. It is further contemplated that the methods of the present disclosure may further include determining the expression levels of at least three response indicator genes, or their expression products. It is contemplated that the methods of the present disclosure may further include determining the expression levels of at least four response indicator genes, or their expression products. It is contemplated that the methods of the present disclosure may further include determining the expression levels of at least five response indicator genes, or their expression products. It is contemplated that the methods of the present disclosure may further include determining the expression levels of at least six response indicator genes, or their expression products. It is contemplated that the methods of the present disclosure may further include determining the expression levels of at least seven response indicator genes, or their expression products. It is contemplated that the methods of the present disclosure may further include determining the expression levels of at least eight response indicator genes, or their expression products. It is contemplated that the methods of the present disclosure may further include determining the expression levels of at least nine response indicator genes, or their expression products. The methods may involve determination of the expression levels of at least ten (10) or at least fifteen (15) of the response indicator genes, or their expression products.

The expression level, or normalized expression level, of the response indicator gene, or its expression product, is used to predict the likelihood of a positive response. In an embodiment of the invention, a likelihood score (e.g., a score predicting a likelihood of a positive response to treatment with a platinum-based chemotherapy drug) can be calculated based on the expression level or normalized expression level. A score may be calculated using weighted values based on the expression level or normalized expression level of a response indicator gene and its contribution to response to a platinum-based chemotherapy drug.

In an embodiment of the invention, the expression product of the response indicator gene to be assayed or measured is an RNA transcript. In one aspect, the RNA transcripts are fragmented. In another embodiment, the expression product is a polypeptide. Determining the expression level of one or more response indicator gene products may be accomplished by, for example, a method of gene expression profiling. The method of gene expression profiling may be, for example, a PCR-based method. The expression level of said genes can be determined, for example, by RT-PCR (reverse transcriptase PCR), quantitative RT-PCR (qRT-PCR), or other PCR-based methods, immunohistochemistry, proteomics techniques, an array-based method, or any other methods known in the art or their combination.

The tumor sample may be, for example, a tissue sample containing cancer cells, or portion(s) of cancer cells, where the tissue can be fixed, paraffin-embedded or fresh or frozen tissue. For example, the tissue may be from a biopsy (fine needle, core or other types of biopsy) or obtained by fine needle aspiration, or by obtaining body fluid containing a cancer cell, e.g. urine, blood, etc. In an embodiment of the invention, the tumor sample is obtained from a patient with colorectal cancer. In a specific embodiment of the invention, the patient has stage II (Dukes B) or stage III (Dukes C) colorectal cancer.

In another embodiment of the invention, the platinum-based chemotherapy drug is selected from cisplatin, carboplatin, and oxaliplatin. In a particular embodiment, the platinum-based chemotherapy drug is oxaliplatin. Oxaliplatin may be provided alone, or in combination, with one or more additional anti-cancer agents. In a specific embodiment, oxaliplatin is provided in combination with fluorouracil (5-FU) and leucovorin.

### BRIEF DESCRIPTION OF THE DRAWINGS

FIGS. 1A-1B show the quality control metrics of the siRNA screen. FIG. 1A shows the deviation between biological replicates of the siRNA screen by plotting the log₂ fold shift IC₅₀ of the first replicate against the log₂ fold shift IC₅₀ of the second replicate, and the R² value is as indicated. FIG. 1B shows the Z′-factor for each plate in the siRNA screen.

FIGS. 2A-2B show the identification and functional classification of genes modulating HCT116 tumor cell sensitivity to oxaliplatin. FIG. 2A shows the results of a 500-gene siRNA screen for genes that modulate sensitivity to oxaliplatin. The median log₂ fold shift in the IC₅₀ of oxaliplatin following siRNA-treatment is plotted for each gene in the screen. Genes with a median IC₅₀ shift>median IC₅₀±3 MAD and an RSA P value<0.05 are indicated in large dark circles above 0 log₂ fold shift IC₅₀ (increased resistance to oxaliplatin) or large dark circles below 0 log₂ fold shift IC₅₀ (increased sensitivity to oxaliplatin). FIG. 2B groups the genes according to biological process using PANTHER®.

FIGS. 3A-3B show the functional classification of genes from the siRNA screen into statistically significant gene subsets. FIG. 3A shows the classification of genes from the siRNA screen based on gene ontology (GO) biological processes. FIG. 3B shows the classification of genes from the siRNA screen based on the Ingenuity® Pathway Analysis. Threshold for statistical significance is indicated as a horizontal dotted line (p<0.05).

FIGS. 4A-4B show the validation of siRNA knockdown and cDNA overexpression. FIG. 4A shows the validation of decreased mRNA following transfection of HCT116 cells with siRNAs targeting the genes identified in the siRNA screen. Plotted is mean±SEM (n=3) fraction of mRNA remaining relative to media-alone treated cells. FIG. 4B shows the validation of increased mRNA following transfection of HCT116 cells with full-length LTBR and TMEM30A open reading frames cloned into pCMV-XL4. Plotted is mean±SEM (n=3) fraction of mRNA relative to pCMV-XL4 (empty vector) alone transfected cells.

FIGS. 5A-5C show the validation of genes identified in the siRNA screen for genes regulating sensitivity or resistance to oxaliplatin. The effect of siRNA-silencing or cDNA overexpression on the IC₅₀ of oxaliplatin was expressed as the log₂ fold-shift of the mean IC₅₀ of siRNA-treated (or cDNA-overexpressing) cells relative to the mean IC₅₀ of non-silencing siRNA control-treated (or vector-alone) cells. Cell viability was assayed and IC₅₀ of oxaliplatin was calculated 72 hrs after cDNA transfection and addition of an 11-point, 2-fold serial dilution of oxaliplatin (50 μM maximum). Data represent mean±SEM (n=3). FIG. 5A shows siRNA-silencing of 12 genes from the primary screen in the HCT116 tumor cell line with ON-TARGETplus® siRNAs, each containing pools of 4 siRNAs per target gene. FIG. 5B shows the siRNA-silencing of selected genes using the SW480 tumor cell line. FIG. 5C shows the effect of cDNA overexpression of full-length LTBR and TMEM30A on the IC₅₀ of oxaliplatin.

FIGS. 6A-6C show functional analyses of genes modulating sensitivity to oxaliplatin. FIG. 6A shows increased levels of DNA damage, as determined by quantification of apurinic/apyrimidinic sites (as % of non-silencing siRNA-treated cells), in CUL4B- and NHEJ1-silenced HCT 116 tumor cells. Cells were transfected, treated with 1.56 μM oxaliplatin, and DNA damage was measured after 72 hr. Dashed line indicates 100% of control. Data represent mean±SEM (n=3); *, P<0.05. FIG. 6B shows hierarchical clustering of relative activities of pathway signaling nodes in cells with altered sensitivity to oxaliplatin. The heat map indicates the normalized log₂ ratio of the phosphorylation levels of AKT1 (Ser437), MEK1 (Ser217/222), p38 MAPK (Thr 180/Tyr182), STAT3 (Tyr705), and NFκB p65 (Ser536) in test siRNA-treated cells (+1.56 μM oxaliplatin) relative to non-silencing siRNA-treated cells (+1.56 μM oxaliplatin), as assessed by quantitative analysis using a sandwich ELISA with epitope-specific antibodies 72 hr post transfection and addition of oxaliplatin. FIG. 6C shows hierarchical clustering of relative activities of key apoptotic regulators, in cells with altered sensitivity to oxaliplatin. The heat map indicates the normalized log₂ ratio of the phosphorylation levels of p53 (Ser15), and Bad (Ser112), as well as the cleavage status of PARP and Caspase-3 in test siRNA-treated cells (+1.56 μM oxaliplatin) relative to non-silencing siRNA-treated cells (+1.56 μM oxaliplatin), as assessed by quantitative analysis using a sandwich ELISA with epitope-specific antibodies 72 hr post transfection and addition of oxaliplatin. Color bar indicates log₂ of relative activity (phosphorylation or cleavage).

FIG. 7 shows alterations in cell cycle distribution in cells with altered sensitivity to oxaliplatin. X-axis indicates DNA content (as determined by propidium iodide staining), and Y-axis indicates cell count. Coding indicates G1, S, or G2/M phases of the cell cycle. Percentages of each stage are indicated (first percentage, G1; second percentage, S; third percentage, G2/M). Cells were transfected, treated with 1.56 μM oxaliplatin, and processed for FACS after 72 hr.

FIG. 8 shows a network modeling of the genes in the siRNA screen and shows multiple pathways linked to oxaliplatin sensitivity. Networks of interacting proteins were identified using Ingenuity Pathway Analysis. CDKN1A, KPNA2, SUMO1, and TP53 are genes that exhibited increased resistance to oxaliplatin. The remaining genes shown with filled shapes exhibited increased sensitivity to oxaliplatin.

### DETAILED DESCRIPTION

### Definitions

Unless defined otherwise, technical and scientific terms used herein have the same meaning as commonly understood by one of ordinary skill in the art to which this invention belongs. Singleton et al., Dictionary of Microbiology and Molecular Biology, 2^{nd} ed., J. Wiley & Sons (New York, N.Y. 1994), and March, Advanced Organic Chemistry Reactions, Mechanisms and Structure, 4th ed., J. Wiley & Sons (New York, N.Y. 1992), provide one skilled in the art with a general guide to many of the terms used in the present application.

One skilled in the art will recognize many methods and materials similar or equivalent to those described herein that may be used in the practice of the present invention. Indeed, the present invention is in no way limited to the methods and materials described herein. For purposes of the invention, the following terms are defined below.

As used herein, the term “amplicon” refers to a piece of DNA that has been synthesized using an amplification technique, such as the polymerase chain reaction (PCR) and ligase chain reaction.

The term “anti-cancer agent” as used herein refers to any molecule, compound, chemical, or composition that has an anti-cancer effect, such as a “positive response” as defined below. Anti-cancer agents include, without limitation, chemotherapeutic agents, radiotherapeutic agents, cytokines, anti-angiogenic agents, apoptosis-inducing agents or anti-cancer immunotoxins, such as antibodies. Examples of anti-cancer agents include, without limitation, methotrexate, taxol, mercaptopurine, thioguanine, hydroxyurea, cytarabine, cyclophosphamide, ifosfamide, nitrosoureas, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, fluorouracil (5-FU), and leucovorin. Other anti-cancer agents are known in the art. In an embodiment of the invention, the anti-cancer agent is 5-FU and leucovorin.

The terms “assay” or “assaying” as used herein refer to performing a quantitative or qualitative analysis of a component in a sample. The terms include laboratory or clinical observations, and/or measuring the level of the component in the sample.

The terms “cancer” and “cancerous” as used herein, refer to or describe the physiological condition that is typically characterized by unregulated cell growth. Examples of cancer in the present application include cancer of the gastrointestinal tract, such as invasive colorectal cancer or Stage II (Dukes B) or Stage III (Dukes C) colorectal cancer.

The term “co-expressed” as used herein refers to a statistical correlation between the expression level of one gene and the expression level of another gene. Pairwise co-expression may be calculated by various methods known in the art, e.g., by calculating Pearson correlation coefficients or Spearman correlation coefficient. Co-expressed gene cliques may also be identified using a graph theory. An analysis of co-expression may be calculated using normalized expression data.

The terms “colon cancer” and “colorectal cancer” are used interchangeably herein and refer in the broadest sense to (1) all stages and all forms of cancer arising from epithelial cells of the large intestine and/or rectum and/or (2) all stages and all forms of cancer affecting the lining of the large intestine and/or rectum. In the staging systems used for classification of colorectal cancer, the colon and rectum are treated as one organ.

The term “correlates” or “correlating” as used herein refers to a statistical association between instances of two events, where events may include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases. The present invention provides genes and gene subsets, the expression levels of which are correlated with a particular outcome measure, such as between the expression level of a gene and the likelihood of a positive response to treatment with a drug. For example, the increased expression level of a gene product may be positively correlated with a likelihood of a good clinical outcome for the patient, such as an increased likelihood of long-term survival without recurrence and/or a positive response to a chemotherapy, and the like. Such a positive correlation may be demonstrated statistically in various ways, e.g. by a low hazard ratio. In another example, the increased expression level of a gene product may be negatively correlated with a likelihood of good clinical outcome for the patient. In this case, for example, the patient may have a decreased likelihood of long-term survival without recurrence of the cancer and/or a positive response to a chemotherapy, and the like. Such a negative correlation indicates that the patient likely has a poor prognosis or will respond poorly to a chemotherapy, and this may be demonstrated statistically in various ways, e.g., a high hazard ratio.

The term “Ct” as used herein refers to threshold cycle, the cycle number in quantitative polymerase chain reaction (qPCR) at which the fluorescence generated within a reaction well exceeds the defined threshold, i.e. the point during the reaction at which a sufficient number of amplicons have accumulated to meet the defined threshold.

The term “expression level” as used herein refers to qualitative or quantitative determination of an expression product or gene product. Expression level may be determined for the RNA expression level of a gene or for the polypeptide expression level of a gene. The term “normalized” expression level as used herein refers to an expression level of a response indicator gene relative to the level of an expression product of a reference gene(s), which might be all measured expression products in the sample, a single reference expression product, or a particular set of expression products. A gene exhibits an “increased expression level” when the expression level of an expression product is higher in a first sample, such as in a clinically relevant subpopulation of patients (e.g., patients who are responsive to a platinum-based chemotherapy drug), than in a second sample, such as in a related subpopulation (e.g., patients who are not responsive to the platinum-based chemotherapy drug). Similarly, a gene exhibits an “increased normalized expression level” when the normalized expression level of an expression product is higher in a first sample, such as in a clinically relevant subpopulation of patients (e.g., patients who are responsive to a platinum-based chemotherapy drug), than in a second sample, such as in a related subpopulation (e.g., patients who are not responsive to the platinum-based cheMotherapy drug).

In the context of an analysis of an expression level of a gene in tissue obtained from an individual subject, a gene exhibits “increased expression,” or “increased normalized expression” when the expression level or normalized expression level of the gene in the subject trends toward, or more closely approximates, the expression level or normalized expression level characteristic of a clinically relevant subpopulation of patients.

Thus, for example, when the gene analyzed is a gene that shows increased expression in responsive subjects as compared to non-responsive subjects, then “increased expression” or “increased normalized” expression level of a given gene can be described as being positively correlated with a likelihood of a positive response to a platinum-based chemotherapy drug. If the expression level of the gene in the individual subject trends toward a level of expression characteristic of a responsive subject, then the gene expression level supports a determination that the individual subject is more likely to be a responder. If the expression level of the gene in the individual subject trends toward a level of expression characteristic of a non-responsive subject, then the gene expression level supports a determination that the individual subject is more likely to be a non-responder.

Similarly, where the gene analyzed is a gene that is increased in expression in non-responsive patients as compared to responsive patients, then “increased expression” or “increased normalized” expression level of a given gene can be described as being negatively correlated with a likelihood of a positive response to a platinum-based chemotherapy drug. If the expression level of the gene in the individual sample trends toward a level of expression characteristic of a non-responsive subject, then the gene expression level supports a determination that the individual patient will more likely to be non-responsive. If the expression level of the gene in the individual sample trends toward a level of expression characteristic of a responsive subject, then the gene expression level supports a determination that the individual patient will more likely to be responsive.

Of course, the same meaning can be derived by changing the terms “increased” with “decreased” as long as the association of the relationship between the gene expression level and likelihood of a positive response remains the same. For instance, the phrase “increased expression level of a gene is positively correlated with a likelihood of a positive response” can be rephrased as “decreased expression level of a gene is negatively correlated with a likelihood of a positive response” to mean the same thing. It can also be rephrased to “increased expression level of a gene is negatively correlated with a decreased likelihood of a positive response” to mean the same thing.

The term “expression product” or “gene product” are used herein to refer to the RNA transcription products (transcripts) of a gene, including mRNA, and the polypeptide translation products of such RNA transcripts. An expression product may be, for example, an unspliced RNA, an mRNA, a splice variant mRNA, a microRNA, a fragmented RNA, a polypeptide, a post-translationally modified polypeptide, a splice variant polypeptide, etc.

The term “long-term” survival is used herein to refer to survival for a particular time period. In an embodiment of the invention, the time period of long-term survival is for at least 3 years. In another embodiment, the time period of long-term survival is for at least 5 years.

The term “measuring” as used herein refers to performing a physical act of determining the dimension, quantity, or capacity of a component in a sample.

The term “microarray” as used herein refers to an ordered arrangement of hybridizable array elements, e.g., oligonucleotide or polynucleotide probes, on a substrate.

The term “polynucleotide” generally refers to any polyribonucleotide or polydeoxyribonucleotide, which may be unmodified RNA or DNA or modified RNA or DNA. Thus, for instance, polynucleotides as used herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” also includes DNAs (including cDNAs) and RNAs and those that contain one or more modified bases. Thus, DNAs or RNAs with backbones modified for stability or for other reasons, are “polynucleotides” as that term is used herein. Moreover, DNAs or RNAs comprising unusual basbs, such as inosine, or modified bases, such as tritiated bases, are included within the term “polynucleotides” as used herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA/DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “primer” or “oligonucleotide primer” as used herein, refers to an oligonucleotide that acts to initiate synthesis of a complementary nucleic acid strand when placed under conditions in which synthesis of a primer extension product is induced, e.g., in the presence of nucleotides and a polymerization-inducing agent such as a DNA or RNA polymerase and at suitable temperature, pH, metal ion concentration, and salt concentration. Primers are generally of a length compatible with their use in synthesis of primer extension products, and can be in the range of between about 8 nucleotides and about 100 nucleotides (nt) in length, such as about 10 nt to about 75 nt, about 15 nt to about 60 nt, about 15 nt to about 40 nt, about 18 nt to about 30 nt, about 20 nt to about 40 nt, about 21 nt to about 50 nt, about 22 nt to about 45 nt, about 25 nt to about 40 nt, and so on, e.g., in the range of between about 18 nt and about 40 nt, between about 20 nt and about 35 nt, between about 21 and about 30 nt in length, inclusive, and any length between the stated ranges. Primers can be in the range of between about 10-50 nucleotides long, such as about 15-45, about 18-40, about 20-30, about 21-25 nt and so on, and any length between the stated ranges. In some embodiments, the primers are not more than about 10, 12, 15, 20, 21, 22, 23, 24, 25, 26, 27, 28, 29, 30, 35, 40, 45, 50, 55, 60, 65, or 70 nucleotides in length. In this context, the term “about” may be construed to mean 1, 2, 3, 4, 5, 6, 7, 8, 9, 10, 11, 12, 13, 14, 15, 16, 17, 18, 19 or 20 more nucleotides either 5′ or 3′ from either termini or from both termini.

Primers are in many embodiments single-stranded for maximum efficiency in amplification, but may alternatively be double-stranded. If double-stranded, the primer is in many embodiments first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the covalent addition of bases at its 3′ end.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

As used herein, the term “probe” or “oligonucleotide probe”, used interchangeably herein, refers to a structure comprised of a polynucleotide, as defined above, that contains a nucleic acid sequence complementary to a nucleic acid sequence present in the target nucleic acid analyte (e.g., a nucleic acid amplification product). The polynucleotide regions of probes may be composed of DNA, and/or RNA, and/or synthetic nucleotide analogs. Probes are generally of a length compatible with their use in specific detection of all or a portion of a target sequence of a target nucleic acid, and are in many embodiments in the range of between about 8 nt and about 100 nt in length, such as about 8 to about 75 nt, about 10 to about 74 nt, about 12 to about 72 nt, about 15 to about 60 nt, about 15 to about 40 nt, about 18 to about 30 nt, about 20 to about 40 nt, about 21 to about 50 nt, about 22 to about 45 nt, about 25 to about 40 nt in length, and so on, e.g., in the range of between about 18-40 nt, about 20-35 nt, or about 21-30 nt in length, and any length between the stated ranges. In some embodiments, a probe is in the range of between about 10-50 nucleotides long, such as about 15-45, about 18-40, about 20-30, about 21-28, about 22-25 and so on, and any length between the stated ranges. In some embodiments, the probes are not more than about 10, 12, 15, 20, 21, 22, 23, 24, 25, 26, 27, 28, 29, 30, 35, 40, 45, 50, 55, 60, 65, or 70 nucleotides in length. In this context, the term “about” may be construed to mean 1, 2, 3, 4, 5, 6, 7, 8, 9, 10, 11, 12, 13, 14, 15, 16, 17, 18, 19 or 20 more nucleotides either 5′ or 3′ from either termini or from both termini.

As used herein, the term “pathology” of cancer includes all phenomena that comprise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes.

The term “platinum-based chemotherapy drug” as used herein refers to a molecule or a composition comprising a molecule containing a coordination complex comprising the chemical element platinum and useful as a chemotherapy drug. Platinum-based chemotherapy drugs generally act by inhibiting DNA synthesis and have some alkylating activity. Examples of platinum-based chemotherapy drugs include cisplatin, carboplatin, and oxaliplatin. Platinum-based chemotherapy drugs encompass those that are currently being used as part of a chemotherapy regimen, those that are currently in development, and those that may be developed in the future. The platinum-based chemotherapy drug may be administered as a monotherapy, or in combination with other anti-cancer agents, or as prodrugs, or together with local therapies such as surgery and radiation, or as adjuvant or neoadjuvant chemotherapy, or as part of a multimodal approach to the treatment of neoplastic disease. For example, oxaliplatin may be administered alone, or in combination with fluorouracil (5-FU) and/or leucovorin for the treatment of colorectal cancer.

The term “positive response” as used herein refers to a favorable response to a drug as opposed to an unfavorable response, such as adverse events. A positive response may include, without limitation, (1) inhibition, to some extent, of tumor growth, including slowing down to complete growth arrest; (2) reduction in the number of tumor cells; (3) reduction in tumor size; (4) inhibition (i.e., reduction, slowing down or complete cessation) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. In individual patients, a positive response can be expressed in terms of a number of clnical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor, relief, to some extent, of one or more symptoms associated with the tumor, increase in the length of survival following treatment; and/or decreased mortality at a given point of time following treatment. Continued increase in tumor size and/or cancer cell number and/or tumor metastasis is indicative of lack of a positive response to treatment.

In a population, a positive response of a drug can be evaluated on the basis of one or more endpoints. For example, analysis of overall response rate (ORR) classifies as responders those patients who experience CR or PR after treatment with a drug. Analysis of disease control (DC) classifies as responders those patients who experience CR, PR or SD after treatment with drug.

The term “progression free survival” as used herein refers to the time interval from treatment of the patient until the progression of cancer or death of the patient, whichever occurs first.

The term “responder” as used herein refers to a patient who has cancer, and who exhibits a positive response following treatment with a platinum-based chemotherapy drug.

The term “non-responder” as used herein refers to a patient who has cancer, and who has not shown a positive response following treatment with a platinum-based chemotherapy drug.

The term “prediction” is used herein to refer to the likelihood that a cancer cell or a cancer patient will have a particular response to treatment, whether positive or negative. In the context of a cancer patient, “prediction” refers to a particular response to treatment following surgical removal of the primary tumor. For example, treatment could include chemotherapy.

The predictive methods of the present invention can be used clinically to make treatment decisions by choosing the most appropriate treatment modalities for any particular patient. The predictive methods of the present invention are useful tools in predicting if a patient is likely to exhibit a positive response to a treatment regimen, such as chemotherapy, surgical intervention, or both.

The term “reference gene” as used herein refers to a gene whose expression level can be used to compare the expression level of a gene product in a test sample. In an embodiment of the invention, reference genes include housekeeping genes, such as beta-globin, alcohol dehydrogenase, or any other gene, the expression of which does not vary depending on the disease status of the cell containing the gene. In another embodiment, all of the assayed genes or a large subset thereof may serve as reference genes.

The term “response indicator gene” as used herein refers to a gene, the expression of which correlates positively or negatively with a positive response to a platinum-based chemotherapy drug, such as oxaliplatin. The expression of a response indicator gene may be determined by assaying or measuring the expression level of an expression product of the response indicator gene.

The term “RNA transcript” as used herein refers to the RNA transcription product of a gene, including, for example, mRNA, an unspliced RNA, a splice variant mRNA, a microRNA, and a fragmented RNA.

Unless indicated otherwise, each gene name used herein corresponds to the Official Symbol assigned to the gene and provided by Entrez Gene (URL: www.ncbi.nlm.nih.gov/sites/entrez) as of the filing date of this application.

The term “tumor sample” as used herein refers to a sample comprising tumor material obtained from a cancerous patient. The term encompasses tumor tissue samples, for example, tissue obtained by surgical resection and tissue obtained by biopsy, such as for example, a core biopsy or a fine needle biopsy. Additionally, the term “tumor sample” encompasses a sample comprising tumor cells obtained from sites other than the primary tumor, e.g., circulating tumor cells. The term also encompasses cells that are the progeny of the patient's tumor cells, e.g. cell culture samples derived from primary tumor cells or circulating tumor cells. The term further encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer (e.g., colorectal cancer or other cancer referenced herein), including those who have undergone or are candidates for resection (surgery) to remove cancerous tissue (e.g., cancerous colorectal tissue or other cancer referenced herein).

As used herein, the term “surgery” applies to surgical methods undertaken for removal of cancerous tissue, including resection, laparotomy, colectomy (with or without lymphadenectomy), ablative therapy, endoscopic removal, excision, dissection, and tumor biopsy/removal. The tumor tissue or sections used for gene expression analysis may have been obtained from any of these methods.

The terms “threshold” or “thresholding” refer to a procedure used to account for non-linear relationships between gene expression measurements and clinical response as well as to further reduce variation in reported patient scores. When thresholding is applied, all measurements below or above a threshold are set to that threshold value. Non-linear relationship between gene expression and outcome could be examined using smoothers or cubic splines to model gene expression in Cox PH regression on recurrence free interval or logistic regression on recurrence status. Variation in reported patient scores could be examined as a function of variability in gene expression at the limit of quantitation and/or detection for a particular gene.

The terms “treatment” and “treating” refer to administering or contacting an agent, or carrying out a procedure (e.g., radiation, a surgical procedure, etc.), for the purpose of obtaining an effect. In a subject, the effect may be prophylactic in terms of completely or partially preventing a disease or symptom thereof and/or may be therapeutic in terms of effecting a partial or complete cure for a disease and/or symptoms of the disease. The terms cover any treatment of a disease in a mammal, particularly in a human, and includes: (a) preventing the disease or a symptom of a disease from occurring in a subject that may be predisposed to the disease but has not yet been diagnosed as having it (e.g., including diseases that may be associated with or caused by a primary disease); (b) inhibiting the disease, i.e., arresting its development; and (c) relieving the disease, i.e., causing regression of the disease.

The term “tumor” as used herein, refers to all neoplastic cell growth and proliferation, whether malignant or benign, and all pre-cancerous and cancerous cells and tissues.

Two main staging systems are known in the art for colorectal cancer. According to the tumor, node, metastasis (TNM) staging system of the American Joint Committee on Cancer (AJCC) (Green et al. (eds.), ‘AJCC Cancer Staging Manual, 6^{th} ed., Springer: New York, N.Y., 2002), the various stages of colorectal cancer are defined as follows:

Tumor: T1: tumor invades submucosal; T2: tumor invades muscularis propria; T3: tumor invades through the muscularis propria into the subserose, or into the pericolic or perirectal tissues; T4: tumor directly invades and/or perforates other organs or structures.

Node: N0: no regional lymph node metastasis; N1: metastasis in 1 to 3 regional lymph nodes; N2: metastasis in 4 or more regional lymph nodes.

Metastasis: M0: no distant metastasis; M1: distant metastasis present.

Stage groupings: Stage I: T1, N0, M0 or T2, N0, M0; Stage II: T3, N0, M0 or T4, N0, M0; Stage III: any T, N1-2, M0; Stage IV: any T, any N, M1.

According to the Modified Duke Staging System, the various stages of colorectal cancer are defined as follows:

Stage A: the tumor penetrates into the mucosa of the bowel wall but not further. Stage B: tumor penetrates into and through the muscularis propria of the bowel wall. Stage C: tumor penetrates into but not through the muscularis propria of the bowel wall and there is pathologic evidence of colorectal cancer in the lymph nodes; or tumor penetrates into and through the muscularis propria of the bowel wall and there is pathologic evidence of cancer in the lymph nodes. Stage D: tumor has spread beyond the confines of the lymph nodes, into other organs, such as the liver, lung, or bone.

The term “computer-based system”, as used herein refers to the hardware means, software means, and data storage means used to analyze information. The minimum hardware of a patient computer-based system comprises a central processing unit (CPU), input means, output means, and data storage means. A skilled artisan can readily appreciate that many of the currently available computer-based system are suitable for use in the present invention and may be programmed to perform the specific measurement and/or calculation functions of the present invention.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” or “computing means” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

Before the present invention and specific exemplary embodiments of the invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

As used herein and in the appended claims, the singular forms “a,” “an,” and “the” include plural referents unless the context clearly dictates otherwise. Thus, for example, reference to “a reference gene” includes a plurality of such genes and reference to “a platinum-based chemotherapy drug” includes reference to one or more platinum-based chemotherapy drug, and so forth.

### DETAILED DESCRIPTION

The practice of the methods and compositions of the present disclosure will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

The present invention provides response indicator genes of platinum-based chemotherapy drugs. These genes are listed in Tables 1-4. The response indicator genes may be further grouped into gene subsets, depending on their known function. For example, the gene subsets may include a “drug resistance group,” “drug transporter group,” “apoptosis group,” “DNA damage repair group,” “cell cycle group,” “p53 pathway group,” and “nucleotide excision repair (NER) group.” Table 1 indicates which gene subset in which each gene may be grouped. The present invention further provides methods for determining genes that co-express with the response indicator genes. The co-expressed genes themselves are useful response indicator genes. The co-expressed genes may be substituted for the response indicator gene with which they co-express.

The present invention provides a number of methods that utilize the response indicator genes and associated information. In a first aspect, the present invention provides a method of determining whether a cancer cell is likely to exhibit a positive response to a platinum-based chemotherapy drug. In another aspect, the present invention provides a method of predicting a likelihood that a patient with cancer will exhibit a positive response to a treatment comprising a platinum-based chemotherapy drug. The methods of the invention comprise assaying or measuring the expression level of the response indicator gene(s) in a sample comprising cancer cells or in a tumor sample, and determining the likelihood of a positive response based on the correlation between the expression level of the response indicator gene(s) and a positive response to the platinum-based chemotherapy drug.

The response indicator genes and associated information provided by the present invention also have utility in the development of therapies to treat cancers and screening patients for inclusion in clinical trials that test the efficacy of platinum-based chemotherapy drugs. The response indicator genes and associated information may further be used to design or produce a reagent that modulates the level or activity of the expression product. Such reagents may include, but are not limited to, an antisense RNA, a small inhibitory RNA (siRNA), a ribozyme, a small molecule, a monoclonal antibody, and a polyclonal antibody.

In various embodiments of the methods of the present invention, various technological approaches are available for assaying or measuring the expression levels of the response indicator genes, including, without limitation, RT-PCR, microarrays, serial analysis of gene expression (SAGE), and nucleic acid sequence, which are described in more detail below.

### Correlating Expression Level of a Response Indicator Gene Product to a Positive Response to a Platinum-Based Chemotherapy Drug

One skilled in the art will recognize that there are many statistical methods that may be used to determine whether there is a correlation between an outcome of interest (e.g., likelihood of survival, likelihood of response to chemotherapy) and expression levels of a gene product as described here. This relationship can be presented as a continuous recurrence score (R^{S}), or patients may be stratified into risk groups (e.g., low, intermediate, high). For example, a Cox proportional hazards regression model may fit to a particular clinical endpoint (e.g., RFI, DFS, OS). One assumption of the Cox proportional hazards regression model is the proportional hazards assumption, i.e. the assumption that effect parameters multiply the underlying hazard. Assessments of model adequacy may be performed including, but not limited to, examination of the cumulative sum of martingale residuals. One skilled in the art would recognize that there are numerous statistical methods that may be used (e.g., Royston and Parmer (2002), smoothing spline, etc.) to fit a flexible parametric model using the hazard scale and the Weibull distribution with natural spline smoothing of the log cumulative hazards function, with effects for treatment (chemotherapy or observation) and R^{S} allowed to be time-dependent. (See, e.g., P. Royston, M. Parmer, Statistics in Medicine 21(15:2175-2197 (2002).)

Many statistical methods may be used to determine if there is a correlation between expression levels of response indicator genes and positive response to treatment. For example, this relationship can be presented as a continuous treatment score (TS), or patients may stratified into benefit groups (e.g., low, intermediate, high). The interaction studied may vary, e.g. standard of care vs. new treatment, or surgery alone vs. surgery followed by chemotherapy. For example, a Cox proportional hazards regression could be used to model the follow-up data, i.e. censoring time to recurrence at a certain time (e.g., 3 years) after randomization for patients who have not experienced a recurrence before that time, to determine if the TS is associated with the magnitude of chemotherapy benefit. One might use the likelihood ratio test to compare the reduced model with RS, TS and the treatment main effect, with the full model that includes RS, TS, the treatment main effect, and the interaction of treatment and TS. A pre-determined p-value cut-off (e.g., p<0.05) may be used to determine significance.

Alternatively, the method of Royston and Parmer (2002) can be used to fit a flexible parametric model using the hazard scale and the Weibull distribution with natural spline smoothing of the log cumulative hazards function, with effects for treatment (chemotherapy or observation), RS, TS and the interaction of TS with treatment, allowing the effects of RS, TS and TS interaction with treatment to be time dependent. To assess relative chemotherapy benefit across the benefit groups, pre-specified cut-points for the RS and TS may be used to define low, intermediate, and high chemotherapy benefit groups. The relationship between treatment and (1) benefit groups; and (2) clinical/pathologic covariates may also be tested for significance. For example, one skilled in the art could identify significant trends in absolute chemotherapy benefit for recurrence at 3 years across the low, intermediate, and high chemotherapy benefit groups for surgery alone or surgery followed by chemotherapy groups. An absolute benefit of at least 3-6% in the high chemotherapy benefit group would be considered clinically significant.

In an exemplary embodiment, power calculations are carried out for the Cox proportional hazards model with a single non-binary covariate using the method proposed by F. Hsieh and P. Lavori, Control Clin Trials 21:552-560 (2000) as implemented in PASS 2008.

Any of the methods described may group the expression levels of response indicator genes. The grouping of genes may be performed at least in part based on knowledge of the contribution of the genes according to physiologic functions or component cellular characteristics, such as in the gene subsets described herein. The formation of groups, in addition, can facilitate the mathematical weighting of the contribution of various expression levels to the recurrence and/or treatment scores. The weighting of a gene group representing a physiological process or component cellular characteristic can reflect the contribution of that process or characteristic to the pathology of the cancer and clinical outcome. Accordingly, the present invention provides gene subsets of the response indicator genes identified herein for use in the methods disclosed herein.

The response indicator genes of platinum-based chemotherapy drugs of the present invention are listed in Tables 1-4. In an embodiment of the invention, increased expression level of one or more genes selected from ATP6V0C, BCL10, BCL2L10, BFAR, BRIP1, CARD6, CCND1, CDC20, CDC25A, CFLAR, CHAF 1A, CRADD, CUL4B, DFFA, E2F2, E2F4, E2F6, GADD45B, HMG20B, IL8, LTBR, MBD2, MBD3, MBD4, MCM3, MCM4, MCM6, MGST3, MPG, MRPL3, MSH4, NHEJ1, OGT, PAICS, PPP2R5C, PRDX4, PTTG1, RAD51L1, RARA, RBM4, RECQL, RRM1, SHFM1, SPO11, TMEM30A, UBE2A, UBE2S, XAB2, and XRCC2 is negatively correlated with a likelihood of a positive response to a platinum-based chemotherapy drug.

In another embodiment of the invention, increased expression level of one or more genes selected from ABL1, APAF1, BAX, CARD4, CASP5, CCT5, CDKN1A, CDKN3, CIDEA, CRIP2, CUL1, CYP1A2, DNMT1, ERCC4, FANCE, GSTT1, GSTZ1, GTF2H5, KPNA2, MRPS12, MSH5, NFKB1, PTEN, SMARCA4, SND1, SOX4, SUMO1, TARS, TNFRSF10A, TNFSF8, TP53, XPC, and XRCC3 is positively correlated with a likelihood of a positive response to a platinum-based chemotherapy drug.

In a specific embodiment of the invention, increased expression level of one or more genes selected from BCL10, BCL2L10, BFAR, BRIP1, CHAF1A, CUL4B, DFFA, IL8, LTBR, MBD2, MBD4, MCM3, MCM4, MCM6, MPG, MSH4, NHEJ1, PRDX4, PTTG1, RAD51 L1, RRM1, SHFM1, and TMEM30A is negatively correlated with a likelihood of a positive response to platinum-based chemotherapy drug, and increased expression level of one or more genes selected from CDKN1A, KPNA2, SUMO1, and TP53 is positively correlated with a likelihood of a positive response to a platinum-based chemotherapy drug.

In a particular embodiment of the invention, the platinum-based chemotherapy drug is oxaliplatin and the response indicator gene(s) is assayed or measured in colorectal cancer cells. Oxaliplatin may be provided in combination with one or more anti-cancer agents, such as 5-FU and leucovorin. The colorectal cancer cells may be a tumor sample obtained from a human patient with colorectal cancer, such as stage II (Dukes B) or stage III (Dukes C) colorectal cancer. In another embodiment, the expression level of the response indicator gene(s) is normalized as described in more detail below.

Thus, in an embodiment of the invention, increased expression level of one or more genes selected from ATP6V0C, BCL10, BCL2L10, BFAR, BRIP1, CARD6, CCND1, CDC20, CDC25A, CFLAR, CHAF1A, CRADD, CUL4B, DFFA, E2F2, E2F4, E2F6, GADD45B, HMG20B, IL8, LTBR, MBD2, MBD3, MBD4, MCM3, MCM4, MCM6, MGST3, MPG, MRPL3, MSH4, NHEJ1, OGT, PAICS, PPP2R5C, PRDX4, PTTG1, RAD51L1, RARA, RBM4, RECQL, RRM1, SHFM1, SPO11, TMEM30A, UBE2A, UBE2S, XAB2, and XRCC2 is negatively correlated with a likelihood of a positive response to oxaliplatin in colorectal cancer cells or in a human patient with colorectal cancer, such as such as stage II (Dukes B) or stage III (Dukes C) colorectal cancer.

In another embodiment of the invention, increased expression level of one or more genes selected from ABL1, APAF1, BAX, CARD4, CASP5, CCT5, CDKN1A, CDKN3, CIDEA, CRIP2, CUL1, CYP1A2, DNMT1, ERCC4, FANCE, GSTT1, GSTZ1, GTF2H5, KPNA2, MRPS12, MSH5, NFKB1, PTEN, SMARCA4, SND1, SOX4, SUMO1, TARS, TNFRSF10A, TNFSF8, TP53, XPC, and XRCC3 is positively correlated with a likelihood of a positive response to oxaliplatin in colorectal cancer cells or in a human patient with colorectal cancer, such as such as stage II (Dukes B) or stage III (Dukes C) colorectal cancer.

In a particular embodiment of the invention, increased expression level of one or more genes selected from BCL10, BCL2L10, BFAR, BRIP1, CHAF1A, CUL4B, DFFA, IL8, LTBR, MBD2, MBD4, MCM3, MCM4, MCM6, MPG, MSH4, NHEJ1, PRDX4, PTTG1, RAD51L1, RRM1, SHFM1, and TMEM30A is negatively correlated with a likelihood of a positive response to oxaliplatin in colorectal cancer cells or in a human patient with colorectal cancer, such as stage II (Dukes B) or stage III (Dukes C) colorectal cancer. In another embodiment, increased expression level of one or more genes selected from CDKN1A, KPNA2, SUMO1, and TP53 is positively correlated with a likelihood of a positive response to oxaliplatin in colorectal cancer cells or in a human patient with colorectal cancer, such as stage II (Dukes B) or stage III (Dukes C) colorectal cancer.

### Methods to Predict Likelihood of a Positive Response to a Platinum-Based Chemotherapy Drug

As described above, a number of response indicator genes were identified. Expression levels or normalized expression levels of these indicator gene products can then be determined in cancer cells or in a tumor sample obtained from an individual patient who has cancer and for whom treatment with a platinum-based chemotherapy drug is being contemplated. Depending on the outcome of the assessment, treatment with a platinum-based chemotherapy drug may be indicated, or an alternative treatment regimen may be indicated.

In carrying out the method of the present invention, cancer cells or a tumor sample is assayed or measured for an expression level of a response indicator gene product(s). The tumor sample can be obtained from a solid tumor, e.g., via biopsy, or from a surgical procedure carried out to remove a tumor; or from a tissue or bodily fluid that contains cancer cells. In an embodiment of the invention, the tumor sample is obtained from a patient with colorectal cancer, such as stage II (Duke's B) or stage III (Duke's C) colorectal cancer. In another embodiment, the expression level of a response indicator gene is normalized relative to the level of an expression product of one or more reference genes. In a particular embodiment of the invention, the platinum-based chemotherapy drug is oxaliplatin. Oxaliplatin may be provided in combination with one or more anti-cancer agents, such as 5-FU and leucovorin

The likelihood of a positive response to treatment with a platinum-based chemotherapy drug in an individual patient is predicted by comparing, directly or indirectly, the expression level or normalized expression level of the response indicator gene in the tumor sample from the individual patient to the expression level or normalized expression level of the response indicator gene in a clinically relevant subpopulation of patients. Thus, as explained above, when the response indicator gene analyzed is a gene that shows increased expression in responsive subjects as compared to non-responsive subjects, then if the expression level of the gene in the individual subject trends toward a level of expression characteristic of a responsive subject, then the gene expression level supports a determination that the individual subject is more likely to be a responder. Similarly, where the response indicator gene analyzed is a gene that is increased in expression in non-responsive patients as compared to responsive patients, then if the expression level of the gene in the individual subject trends toward a level of expression characteristic of a non-responsive subject, then the gene expression level supports a determination that the individual patient will more likely to be non-responsive. Thus, increased expression or increased normalized expression level of a given gene can be described as being positively correlated with a likelihood of a positive response to a platinum-based chemotherapy drug, or as being negatively correlated with a likelihood of a positive response to a platinum-based chemotherapy drug.

It is understood that the expression level or normalized expression level of a response indicator gene from an individual patient can be compared, directly or indirectly, to the expression level or normalized expression level of the response indicator gene in a clinically relevant subpopulation of patients. For example, when compared indirectly, the expression level or normalized expression level of the response indicator gene from the individual patient may be used to calculate a likelihood of a positive response, such as a recurrence score (R^{S}) or treatment score (TS) as described above, and compared to a calculated score in the clinically relevant subpopulation of patients.

It is also understood that it can be useful to measure the expression level of a response indicator gene product at multiple time points, for example, prior to and during the course of treatment with a platinum-based chemotherapy drug. For example, an initial assessment of the likelihood that a patient will respond to treatment with a platinum-based chemotherapy drug can be made prior to initiation of treatment in order to optimize treatment choice.

Development of drug resistance is a well-known phenomenon in chemotherapeutic treatment of cancer patients. As they proliferate, tumor cells can accumulate mutations that confer drug resistance through a variety of mechanisms, including resistance to a platinum-based chemotherapy drug. Tests that utilize the measurement of response indicator genes to assess the likelihood of a positive response can be carried out at time intervals to monitor changes indicative of the onset of drug resistance that may arise from changes in the tumor over time. It is not necessary to know what mutations or changes have taken place in the tumor in order to monitor consequent changes in the gene expression level of response indicator genes and assess the likelihood of a continuing positive response.

### Methods of Assaying Expression Levels of a Gene Product

The methods and compositions of the present disclosure will employ, unless otherwise indicated, conventional techniques of molecular biology (including recombinant techniques), microbiology, cell biology, and biochemistry, which are within the skill of the art. Exemplary techniques are explained in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction” (Mullis et al., eds., 1994).

Methods of gene expression profiling include methods based on hybridization analysis of polynucleotides, methods based on sequencing of polynucleotides, and proteomics-based methods. Exemplary methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription PCT (RT-PCR) (Weis et al., Trends in Genetics 8:263-264 (1992)). Antibodies may be employed that can recognize sequence-specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

### Reverse Transcriptase PCR(RT-PCR)

Typically, mRNA is isolated from a sample. The starting material is typically total RNA isolated from a human tumor, usually from a primary tumor. Optionally, normal tissues from the same patient can be used as an internal control. mRNA can be extracted from a tissue sample, e.g., from a sample that is fresh, frozen (e.g. fresh frozen), or paraffin-embedded and fixed (e.g. formalin-fixed).

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from a tumor sample can be isolated, for example, by cesium chloride density gradient centrifugation.

The sample containing the RNA is then subjected to reverse transcription to produce cDNA from the RNA template, followed by exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

PCR-based methods use a thermostable DNA-dependent DNA polymerase, such as a Taq DNA polymerase. For example, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction product. A third oligonucleotide, or probe, can be designed to facilitate detection of a nucleotide sequence of the amplicon located between the hybridization sites of the two PCR primers. The probe can be detectably labeled, e.g., with a reporter dye, and can further be provided with both a fluorescent dye, and a quencher fluorescent dye, as in a Taqman® probe configuration. Where a Taqman® probe is used, during the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 384-well format on a thermocycler. The RT-PCR may be performed in triplicate wells with an equivalent of 2 ng RNA input per 10 μL-reaction volume. During amplification, laser-induced fluorescent signal is collected in real-time through fiber optics cables for all wells, and detected at the CCD. The system includes software for running the instrument and for analyzing the data.

5′-Nuclease assay data are generally initially expressed as a threshold cycle (“Cₜ”). Fluorescence values are recorded during every cycle and represent the amount of product amplified to that point in the amplification reaction. The threshold cycle (Cₜ) is generally described as the point when the fluorescent signal is first recorded as statistically significant.

To minimize errors and the effect of sample-to-sample variation, RT-PCR is usually performed using an internal standard. The ideal internal standard gene (also referred to as a reference gene) is expressed at a constant level among cancerous and non-cancerous tissue of the same origin (i.e., a level that is not significantly different among normal and cancerous tissues), and is not significantly affected by the experimental treatment (i.e., does not exhibit a significant difference in expression level in the relevant tissue as a result of exposure to chemotherapy). For example, reference genes useful in the methods disclosed herein should not exhibit significantly different expression levels in cancerous colon as compared to normal colon tissue. RNAs most frequently used to normalize patterns of gene expression are mRNAs for the housekeeping genes glyceraldehyde-3-phosphate-dehydrogenase (GAPDH) and β-actin. Exemplary reference genes used for normalization comprise one or more of the following genes: ATP5E, GPX1, PGK1, UBB, and VDAC2. Gene expression measurements can be normalized relative to the mean of one or more (e.g., 2, 3, 4, 5, or more) reference genes. Reference-normalized expression measurements can range from 0 to 15, where a one unit increase generally reflects a 2-fold increase in RNA quantity.

Real time PCR is compatible both with quantitative competitive PCR, where an internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for use in the methods of the present disclosure use fixed, paraffin-embedded tissues as the RNA source. mRNA isolation, purification, primer extension and amplification can be preformed according to methods available in the art. (see, e.g., Godfrey et al. J. Molec. Diagnostics 2: 84-91 (2000); Specht et al., Am. J. Pathol. 158: 419-29 (2001)). Briefly, a representative process starts with cutting about 10 μM thick sections of paraffin-embedded tumor tissue samples. The RNA is then extracted, and protein and DNA are depleted from the RNA-containing sample. After analysis of the RNA concentration, RNA is reverse transcribed using gene specific primers followed by RT-PCR to provide for cDNA amplification products.

### Design of PCR Primers and Probes

PCR primers and probes can be designed based upon exon or intron sequences present in the mRNA transcript of the gene of interest. Primer/probe design can be performed using publicly available software, such as the DNA BLAT software developed by Kent, W. J., Genome Res. 12(4):656-64 (2002), or by the BLAST software including its variations.

Where necessary or desired, repetitive sequences of the target sequence can be masked to mitigate non-specific signals. Exemplary tools to accomplish this include the Repeat Masker program available on-line through the Baylor College of Medicine, which screens DNA sequences against a library of repetitive elements and returns a query sequence in which the repetitive elements are masked. The masked sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Rrawetz S, Misener S (eds) Bioinformatics Methods and Protocols: Methods in Molecular Biology. Humana Press, Totowa, N. J., pp 365-386).

Other factors that can influence PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, and exhibit Tm's between 50 and 80° C., e.g. about 50 to 70° C.

For further guidelines for PCR primer and probe design see, e.g. Dieffenbach, C W. et al, “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

Table 1 provides the GeneBank accession numbers and Entrez ID numbers for each of the response indicator genes of the invention. Based on these sequences, primers, probes, and amplicon sequences can be determined using methods known in the art.

### MassARRAY® System

In MassARRAY-based methods, such as the exemplary method developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derived PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

### Other PCR-Based Methods

Further PCR-based techniques that can find use in the methods disclosed herein include, for example, BeadArray® technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression® (BADGE), using the commercially available LuminexlOO LabMAP® system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003).

### Microarrays

Expression levels of a gene of interest can also be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from mRNA of a sample. As in the RT-PCR method, the source of mRNA typically is total RNA isolated from a tumor sample, and optionally from normal tissue of the same patient as an internal control or cell lines. mRNA can be extracted, for example, from frozen or archived paraffin-embedded and fixed (e.g. formalin-fixed) tissue samples.

For example, PCR amplified inserts of cDNA clones of a gene to be assayed are applied to a substrate in a dense array. Usually at least 10,000 nucleotide sequences are applied to the substrate. For example, the microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After washing under stringent conditions to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et at, Proc. Natl. Acad. Sci. USA 93(2):106-149 (1996)). Microarray analysis can be performed on commercially available equipment, following the manufacturer's protocols, such as by using the Affymetrix GenChip® technology, or Incyte's microarray technology.

### Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

### Gene Expression Analysis by Nucleic Acid Sequencing

Nucleic acid sequencing technologies are suitable methods for analysis of gene expression. The principle underlying these methods is that the number of times a cDNA sequence is detected in a sample is directly related to the relative expression of the mRNA corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). More recently, the advent of “next-generation” sequencing technologies has made DGE simpler, higher throughput, and more affordable. As a result, more laboratories are able to utilize DGE to screen the expression of more genes in more individual patient samples than previously possible. See, e.g., J. Marioni, Genome Research 18(9):1509-1517 (2008); R. Morin, Genome Research 18(4):610-621 (2008); A. Mortazavi, Nature Methods 5(7):621-628 (2008); N. Cloonan, Nature Methods 5(7):613-619 (2008).

Isolating RNA from Body Fluids

Methods of isolating RNA for expression analysis from blood, plasma and serum (see for example, Tsui N B et al. (2002) Clin. Chem. 48, 1647-53 and references cited therein) and from urine (see for example, Boom R et al. (1990) J Clin Microbiol. 28, 495-503 and reference cited therein) have been described.

### Immunohistochemistry

Immunohistochemistry methods are also suitable for detecting the expression levels of genes and applied to the method disclosed herein. Antibodies (e.g., monoclonal antibodies) that specifically bind a gene product of a gene of interest can be used in such methods. The antibodies can be detected by direct labeling of the antibodies themselves, for example, with radioactive labels, fluorescent labels, hapten labels such as biotin, or an enzyme such as horse radish peroxidase or alkaline phosphatase. Alternatively, unlabeled primary antibody can be used in conjunction with a labeled secondary antibody specific for the primary antibody. Immunohistochemistry protocols and kits are well known in the art and are commercially available.

### Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics.

General Description of the mRNA Isolation, Purification and Amplification

The steps of a representative protocol for profiling gene expression using fixed, paraffin-embedded tissues as the RNA source, including mRNA isolation, purification, primer extension and amplification are provided in various published journal articles. (See, e.g., T. E. Godfrey et al., J. Molec. Diagnostics 2: 84-91 (2000); K. Specht et al., Am. J. Pathol. 158: 419-29 (2001), M. Cronin, et al., Am J Pathol 164:35-42 (2004)). Briefly, a representative process starts with cutting a tissue sample section (e.g. about 10 μm thick sections of a paraffin-embedded tumor tissue sample). The RNA is then extracted, and protein and DNA are removed. After analysis of the RNA concentration, RNA repair is performed if desired. The sample can then be subjected to analysis, e.g., by reverse transcription using gene specific promoters followed by PCR.

### Coexpression Analysis

The present invention provides genes that co-express with particular response indicator genes that have been identified as having a correlation with a positive response to a platinum-based chemotherapy drug. To perform particular biological processes, genes often work together in a concerted way, i.e. they are co-expressed. Co-expressed gene groups identified for a disease process like cancer can also serve as response indicator genes. Such co-expressed genes can be assayed in lieu of, or in addition to, assaying of the response indicator gene with which they co-express.

One skilled in the art will recognize that many co-expression analysis methods now known or later developed will fall within the scope and spirit of the present invention. These methods may incorporate, for example, correlation coefficients, co-expression network analysis, clique analysis, etc., and may be based on expression data from RT-PCR, microarrays, sequencing, and other similar technologies. For example, gene expression clusters can be identified using pair-wise analysis of correlation based on Pearson or Spearman correlation coefficients. (See e.g, Pearson K. and Lee A., Biometrika 2:357 (1902); C. Spearman, Amer. J. Psychol. 15:72-101 (1904); J. Myers, A. Well, Research Design and Statistical Analysis, p. 508 (2^{nd} Ed., 2003).) In general, a correlation coefficient of equal to or greater than 0.3 is considered to be statistically significant in a sample size of at least 20. (See e.g., G. Norman, D. Streiner, Biostatistics: The Bare Essentials, 137-138 (3^{rd} Ed. 2007).)

### Reference Normalization

In order to minimize expression measurement variations due to non-biological variations in samples, e.g., the amount and quality of expression product to be measured, raw expression level data measured for a gene product (e.g., cycle threshold (Ct) measurements obtained by qRT-PCR) may be normalized relative to the mean expression level data obtained for one or more reference genes. Examples of reference genes include housekeeping genes, such as GAPDH. Alternatively, all of the assayed genes or a large subset thereof may also concurrently serve as reference genes and normalization can be based on the mean or median signal (Ct) of all of the assayed genes or a subset thereof (often referred to as “global normalization” approach). On a gene-by-gene basis, measured normalized amount of a patient tumor mRNA may be compared to the amount found in a cancer tissue reference set. See e.g., Cronin, M. et al., Am. Soc. Investigative Pathology 164:35-42 (2004). The normalization may be carried out such that a one unit increase in normalized expression level of a gene product generally reflects a 2-fold increase in quantity of expression product present in the sample. For further information on normalization techniques applicable to qRT-PCR data from tumor tissue, see e.g., Silva, S. et al. (2006) BMC Cancer 6, 200; deKok, J. et al. (2005) Laboratory Investigation 85, 154-159.

### Kits of the Invention

The materials for use in the methods of the present invention are suited for preparation of kits produced in accordance with well known procedures. The present invention thus provides kits comprising agents, which may include gene-specific or gene-selective probes and/or primers, for quantitating the expression of the disclosed genes for predicting prognostic outcome or response to treatment. Such kits may optionally contain reagents for the extraction of RNA from tumor samples, in particular, fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microliter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase). Mathematical algorithms used to estimate or quantify prognostic or predictive information are also properly potential components of kits.

### Reports

The methods of this invention are suited for the preparation of reports summarizing the predictions resulting from the methods of the present invention. A “report” as described herein, is an electronic or tangible document that includes elements that provide information of interest relating to a likelihood assessment and its results. A subject report includes at least a likelihood assessment, e.g., an indication as to the likelihood that a cancer patient will exhibit a positive response to a treatment regimen with a platinum-based chemotherapy drug. A subject report can be completely or partially electronically generated, e.g., presented on an electronic display (e.g., computer monitor). A report can further include one or more of: 1) information regarding the testing facility; 2) service provider information; 3) patient data; 4) sample data; 5) an interpretive report, which can include various information including: a) indication; b) test data, where test data can include a normalized level of one or more genes of interest, and 6) other features.

The present invention therefore provides methods of creating reports and the reports resulting therefrom. The report may include a summary of the expression levels of the RNA transcripts, or the expression products of such RNA transcripts, for certain genes in the cells obtained from the patient's tumor tissue. The report may include a prediction that the patient has an increased likelihood of a positive response to treatment with a particular chemotherapy or the report may include a prediction that the subject has a decreased likelihood of a positive response to the chemotherapy. The report may include a recommendation for a treatment modality such as surgery alone or surgery in combination with chemotherapy. The report may be presented in electronic format or on paper.

Thus, in some embodiments, the methods of the present invention further include generating a report that includes information regarding the patient's likelihood of a positive response to chemotherapy, particularly a treatment with a platinum-based chemotherapy drug, such as oxaliplatin. For example, the methods of the present invention can further include a step of generating or outputting a report providing the results of a patient response likelihood assessment, which can be provided in the form of an electronic medium (e.g., an electronic display on a computer monitor), or in the form of a tangible medium (e.g., a report printed on paper or other tangible medium).

A report that includes information regarding the likelihood that a patient will exhibit a positive response to treatment with a platinum-based chemotherapy drug, such as oxaliplatin, is provided to a user. An assessment as to the likelihood that a cancer patient will respond to treatment•with a platinum-based chemotherapy drug, such as oxaliplatin, is referred to as a “response likelihood assessment” or “likelihood assessment.” A person or entity who prepares a report (“report generator”) may also perform the likelihood assessment. The report generator may also perform one or more of sample gathering, sample processing, and data generation, e.g., the report generator may also perform one or more of: a) sample gathering; b) sample processing; c) measuring a level of a response indicator gene expression product(s); d) measuring a level of a reference gene product(s); and e) determining a normalized level of a response indicator gene expression product(s). Alternatively, an entity other than the report generator can perform one or more sample gathering, sample processing, and data generation.

The term “user” or “client” refers to a person or entity to whom a report is transmitted, and may be the same person or entity who does one or more of the following: a) collects a sample; b) processes a sample; c) provides a sample or a processed sample; and d) generates data (e.g., level of a predictive gene expression product(s); level of a reference gene product(s); normalized level of a predictive gene expression product(s)) for use in the likelihood assessment. In some cases, the person or entity who provides sample collection and/or sample processing and/or data generation, and the person who receives the results and/or report may be different persons, but are both referred to as “users” or “clients.” In certain embodiments, e.g., where the methods are completely executed on a single computer, the user or client provides for data input and review of data output. A “user” can be a health professional (e.g., a clinician, a laboratory technician, a physician (e.g., an oncologist, surgeon, pathologist), etc.).

In embodiments where the user only executes a portion of the method, the individual who, after computerized data processing according to the methods of the invention, reviews data output (e.g., results prior to release to provide a complete report, a complete, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

Where government regulations or other restrictions apply (e.g., requirements by health, malpractice, or liability insurance), all results, whether generated wholly or partially electronically, are subjected to a quality control routine prior to release to the user.

### Computer-Based Systems and Methods

The methods and systems described herein can be implemented in numerous ways. In one embodiment of the invention, the methods involve use of a communications infrastructure, for example, the internet. Several embodiments of the invention are discussed below. The present invention may also be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

In an embodiment of the invention, during or after data input by the user, portions of the data processing can be performed in the user-side computing environment. For example, the user-side computing environment can be programmed to provide for defined test codes to denote a likelihood “score,” where the score is transmitted as processed or partially processed responses to the reviewer's computing environment in the form of test code for subsequent execution of one or more algorithms to provide a result and/or generate a report in the reviewer's computing environment. The score can be a numerical score (representative of a numerical value) or a non-numerical score representative of a numerical value or range of numerical values (e.g., “A”: representative of a 90-95% likelihood of a positive response; “High”: representative of a greater than 50% chance of a positive response (or some other selected threshold of likelihood); “Low”: representative of a less than 50% chance of a positive response (or some other selected threshold of likelihood), and the like.

As a computer system, the system generally includes a processor unit. The processor unit operates to receive information, which can include test data (e.g., level of a predictive gene product(s); level of a reference gene product(s); normalized level of a predictive gene product(s); and may also include other data such as patient data. This information received can be stored at least temporarily in a database, and data analyzed to generate a report as described above.

Part or all of the input and output data can also be sent electronically. Certain output data (e.g., reports) can be sent electronically or telephonically (e.g., by facsimile, using devices such as fax back). Exemplary output receiving devices can include a display element, a printer, a facsimile device and the like. Electronic forms of transmission and/or display can include email, interactive television, and the like. In an embodiment of the invention, all or a portion of the input data and/or output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record that may exist in a confidential database as the healthcare facility.

The present invention also contemplates a computer-readable storage medium (e.g., CD-ROM, memory key, flash memory card, diskette, etc.) having stored thereon a program which, when executed in a computing environment, provides for implementation of algorithms to carry out all or a portion of the results of a response likelihood assessment as described herein. Where the computer-readable medium contains a complete program for carrying out the methods described herein, the program includes program instructions for collecting, analyzing and generating output, and generally includes computer readable code devices for interacting with a user as described herein, processing that data in conjunction with analytical information, and generating unique printed or electronic media for that user.

Where the storage medium includes a program that provides for implementation of a portion of the methods described herein (e.g., the user-side aspect of the methods (e.g., data input, report receipt capabilities, etc.)), the program provides for transmission of data input by the user (e.g., via the interne, via an intranet, etc.) to a computing environment at a remote site. Processing or completion of processing of the data is carried out at the remote site to generate a report. After review of the report, and completion of any needed manual intervention, to provide a complete report, the complete report is then transmitted back to the user as an electronic document or printed document (e.g., fax or mailed paper report). The storage medium containing a program according to the invention can be packaged with instructions (e.g., for program installation, use, etc.) recorded on a suitable substrate or a web address where such instructions may be obtained. The computer-readable storage medium can also be provided in combination with one or more reagents for carrying out a response likelihood assessment (e.g., primers, probes, arrays, or such other kit components).

Having described the invention, the same will be more readily understood through reference to the following Examples, which are provided by way of illustration, and are not intended to limit the invention in any way. All citations through the disclosure are hereby expressly incorporated by reference.

### EXAMPLES

### Example 1

In this study, a synthetic-lethal small interfering RNA (siRNA) screen was performed on human CRC cells to identify genes whose loss-of-function (LOF) modulates tumor cell response to oxaliplatin. The screen targeted 500 genes involved in DNA repair, drug transport, metabolism, apoptosis, and regulation of the cell cycle (Table 1). Four unique siRNA duplexes were used over seven different oxaliplatin concentrations per gene. By this method, 82 genes were shown to modify the response to oxaliplatin (Table 2). Of these, 27 genes were chosen for further study whose loss of expression significantly altered the response to oxaliplatin, by either increased sensitivity or increased resistance (Table 3).

Cell Lines and Antibodies

Colon cancer cell lines HCT116 (ATCC# CCL-247) and SW480 (ATCC# CCL-228) were obtained from the American Type Culture Collection (Manassas, Va.), and were maintained in McCoy's 5A media supplemented with 10% fetal bovine serum, 1.5 mM L-glutamine, and 1% Antibiotic-Antimycotic (Invitrogen, Carlsbad, Calif.).

siRNA Screening and Drug Treatments

Four siRNA sequences were selected for each targeted gene from the Whole Human Genome V1.00 and Druggable Genome V2.0® siRNA libraries (Qiagen, Valencia; CA) to create six (6) custom 384-well assay plates. All assay plates included negative control siRNAs (Non-Silencing, All-Star Non-Silencing, and GFP, all from Qiagen), and two positive control siRNAs (UBBs1 and All-Star Cell Death Control® from Qiagen). Selected siRNAs were printed individually into white solid 384-well plates (1 μl of 0.667 μM siRNA per well for a total of 9 ng siRNA) using a Biomek FX® (Beckman Coulter, Brea, Calif.). Lipofectamine 2000® (Invitrogen, Carlsbad, Calif.) was diluted in serum-free McCoy's 5A media and 20 μl was transferred into each well of the 384-well plate containing siRNAs (final ratio of 7.4n1 lipid per ng siRNA). After an incubation period of 30 minutes at room temperature to allow the siRNA and lipid to form complexes, 20 μl of HCT 116 cells (2.5×10⁴ cells/ml) in antibiotic-free McCoy's 5A media were added into each well. Transfected cells were incubated for 24 hours prior to the addition of 10 μl per well of different concentrations of oxaliplatin (35.0, 3.75, 3.0, 2.0, and 1.5 μM) and vehicle control (DMSO) for a total assay volume of 50 μl. Oxaliplatin was obtained from Sigma (St. Louis, Mo.). Cell viability was measured 72 h post drug treatment using the CellTiter-Glo® assay (Promega, Madison, Wis.), measured on an Analyst GT Multimode reader (Molecular Devices, Sunnyvale, Calif.). A repliCate of the screen was also performed, resulting in a total of 56 data points per gene. Cell viability data was normalized to the median value of All-Star NS negative control siRNA and IC₅₀ values were calculated using Prism 5.0® (GraphPad, La Jolla, Calif.).

Statistical Analysis

The effect of siRNA treatment on the IC₅₀ of oxaliplatin was expressed as the log₂ fold-shift of the median IC₅₀ of siRNA-treated cells relative to the median IC₅₀ of non-silencing siRNA control-treated cells. Hits were identified as those with a median IC₅₀ shift greater than the median IC₅₀+3 median absolute deviation (median±3MAD) (Chung, N., et al., Median absolute deviation to improve hit selection for genome-scale RNAi screens. J Biomol Screen, 2008. 13(2): p. 149-58; Birmingham, A., et al., Statistical methods for analysis of high-throughput RNA interference screens. Nat Methods, 2009. 6(8): p. 569-75).

To assign statistical significance to siRNA hits identified from the siRNA screen, collective activities of the 4 individual siRNAs used for each gene were modeled using the redundant siRNA activity (RSA) analysis. Briefly, the normalized, log₂ transformed IC₅₀ shifts of each siRNA were rank ordered. Subsequently, the rank distribution of all siRNAs targeting the same gene was examined and a P value was calculated based on an iterative hypergeometric distribution formula (Konig, R., et al., A probability-based approach for the analysis of large-scale RNAi screens. Nat Methods, 2007. 4(10): p. 847-9). siRNAs with P-values<0.05 were considered as significant. Subsequently, only genes with a median IC₅₀ shift>median IC₅₀±3 MAD and an RSA P value<0.05 were considered robust hits and analyzed further. All other tests of significance were two-sided, and P values<0.05 were considered significant.

Results

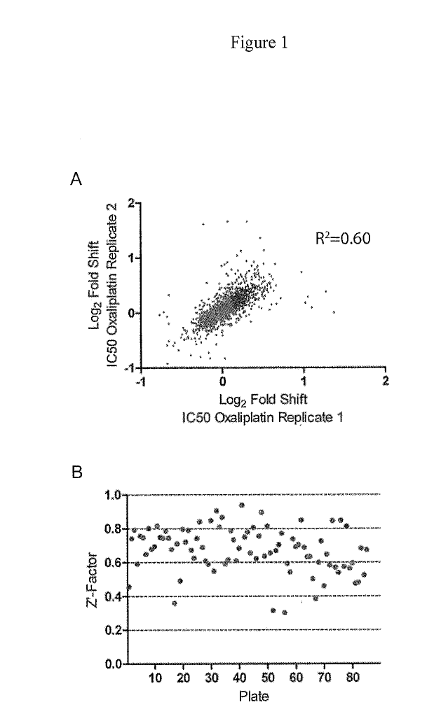

A custom siRNA library targeting 500 genes with putative roles in DNA damage repair, apoptosis, regulation of the cell cycle, drug metabolism and transport, was screened using the colorectal cancer tumor cell line, HCT 116 (Table 1). The siRNA library contained four siRNAs targeting each of the 500 genes, with each siRNA transfected individually. The screen was performed in duplicate, with a non-silencing siRNA negative control. siRNAs were used at 17 nM to reduce off-target effects. Twenty-four hours after transfection, 5 different concentrations of oxaliplatin (35.0, 3.75, 3.0, 2.0, and 1.5 μM) and vehicle control (DMSO) were added and cell viability was measured 72 hours after addition of drug. The deviation between the replicates in the siRNA screen is shown in FIG. 1A by plotting the log₂ fold shift IC₅₀ of the first replicate against the log₂ fold shift IC₅₀ of the second replicate. The R² value was 0.60, as indicated. Moreover, the mean Z′ factor for the screen was 0.67, suggesting that that assay had a robust signal-to-noise ratio (FIG. 1B).

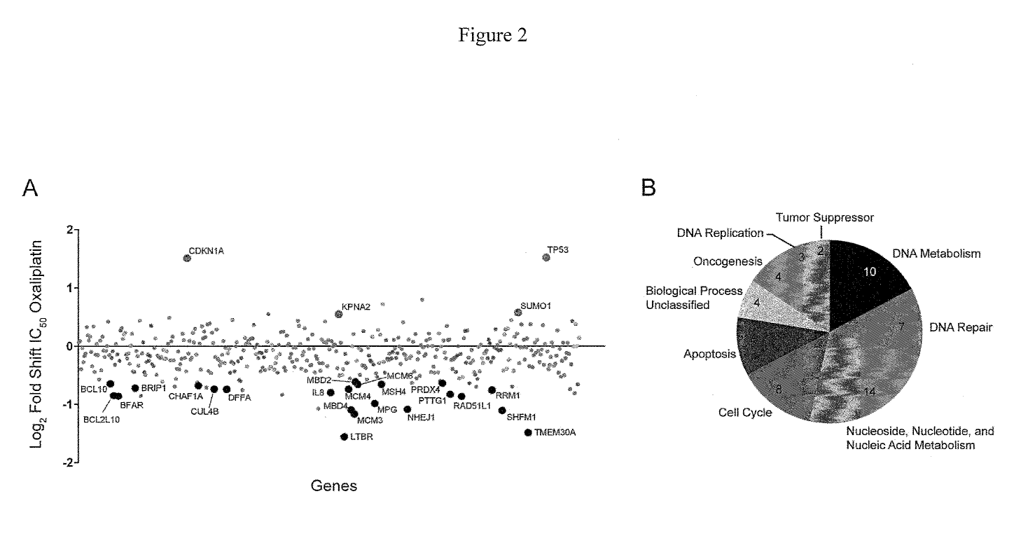

Two criteria were used to limit the discovery of false positives. First, all genes whose silencing shifted the IC₅₀ of oxaliplatin≧±3 median absolute deviations from the median IC₅₀ of oxaliplatin in control cells were identified. This approach (median±k MAD) has been shown to be robust to outliers and effective in controlling the false positive rate in siRNA screens (Chung, N., et al., Median absolute deviation to improve hit selection for genome-scale RNAi screens. J Biomol Screen, 2008. 13(2): p. 149-58; Birmingham, A., et al., Statistical methods for analysis of high-throughput RNA interference screens. Nat Methods, 2009. 6(8): p. 569-75). Second, the collective activities of the 4 individual siRNAs used for each gene were modelled using the redundant siRNA activity (RSA) analysis (Konig, R., et al., A probability-based approach for the analysis of large-scale RNAi screens. Nat Methods, 2007. 4(10): p. 847-9). siRNAs with P-values<0.05 were considered significant (Table 2). 27 genes that satisfied both these criteria were identified (FIG. 2A; Table 3) and analyzed further.

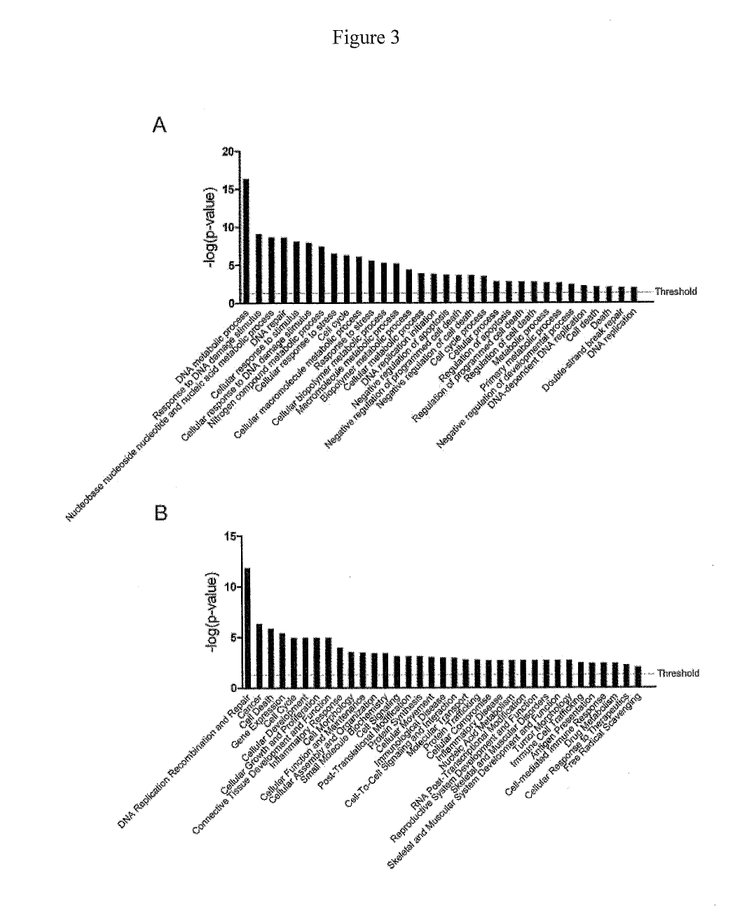

To survey the biological pathways and processes represented by these twenty-seven genes, the PANTHER® database was utilized (Thomas, P. D., et al., PANTHER: a browsable database of gene products organized by biological function, using curated protein family and subfamily classification. Nucleic Acids Res, 2003. 31(1): p. 334-41). The predominant biological process of identified genes is DNA repair and DNA metabolism, as well as nucleoside, nucleotide, and nucleic acid metabolism (FIG. 2B). Additionally, to determine if any of the hits were enriched for known biological processes or canonical pathways in a statistically significant manner, the 27 genes were categorized using Gene Ontology® (GOTermFinder®) (Boyle, E. I., et al., GO::TermFinder—open source software for accessing Gene Ontology information and finding significantly enriched Gene Ontology terms associated with a list of genes. Bioinformatics, 2004. 20(18): p. 3710-5) (FIG. 3A), and Ingenuity® Pathway Analysis (www.ingenuity.com) (FIG. 3B). This analysis also revealed that many of these genes functioned in DNA metabolism, response to DNA damage, cell cycle, and apoptosis. It is noteworthy that there was no significant association with drug metabolism, drug transport, or generalized resistance to chemotherapies amongst these gene hits.

### Example 2

Twelve out of the 27 genes from Example 1 were selected for validation using additional siRNAs. These genes (BRIP1, CDKN1A, CUL4B, LTBR, MBD4, MCM3, NHEJ1, PRDX4, PTTG1, SFHM1, TMEM30A, and TP53) were selected based on the significance analysis and/or functional categorization.

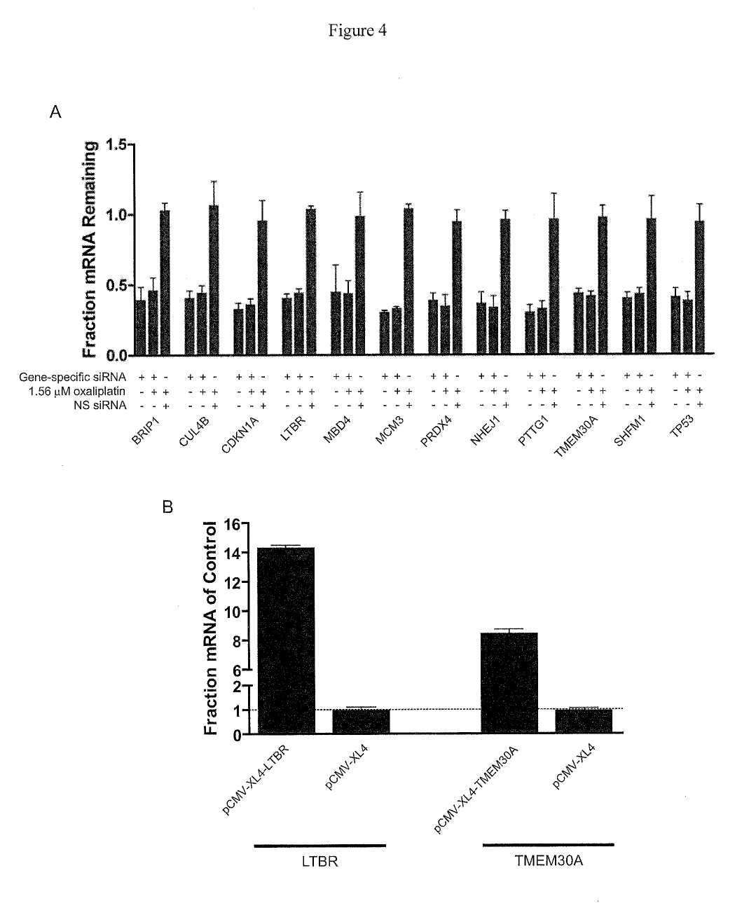

For validation of siRNA hits, ON-TARGETplus® siRNAs (Thermo Scientific, Waltham Mass.), containing pools of 4 siRNAs per gene, were utilized (Table 4). 70 μl of HCT 116 or SW480 cells (1.0×10⁵ cells/ml) were plated in black, clear-bottomed 96-well plates in antibiotic-free McCoy's 5A medium and allowed to adhere overnight. Cells were then transfected with 25 nM siRNA using DharmaFECT® transfection reagent (Thermo Scientific, Waltham, Mass.). Following a 4 hr incubation, 10 μl per well of an 11-point, 2-fold serial dilution of oxaliplatin (50 μM maximum) was then added, for a total assay volume of 100 μl. Assays were performed in triplicate, with ON-TARGETplus Non-Targeting siRNA® (Thermo Scientific, Waltham, Mass.) as a negative control, with biological replicates. Cell viability was measured 72 h later using the CellTiter 96® AQueous One Solution Cell Proliferation Assay (Promega, Madison, Wis.), and IC₅₀ values calculated using Prism 5.0® (GraphPad, La Jolla, Calif.). siRNA knockdown was validated by qRT-PCR using the High-Capacity cDNA Reverse Transcription Kite (Life Technologies, Carlsbad, Calif.) and qPCR using the 7900 HT Fast Real-Time PCR System® (Life Technologies, Carlsbad, Calif.) with gene-specific primers (ABI, Carlsbad, Calif.). (FIG. 4A).

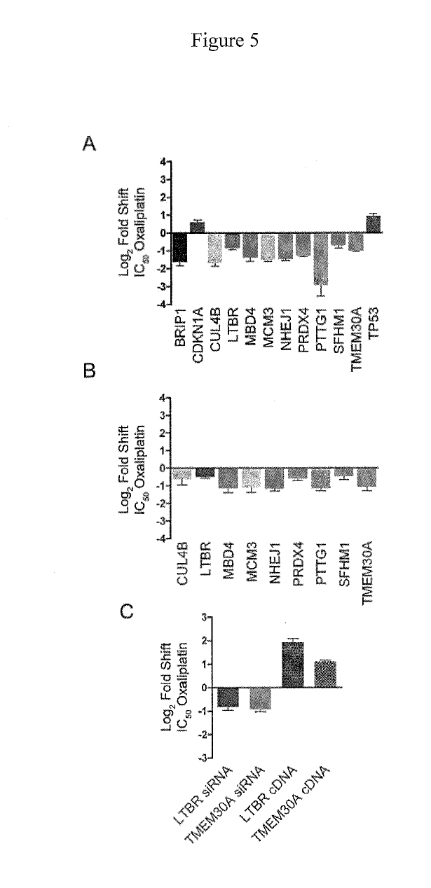

The retested genes were considered to be validated if the resulting IC₅₀ of oxaliplatin shifted >50% from the IC₅₀ of oxaliplatin in cells treated with non-silencing siRNAs. All twelve of the genes selected for validation exceeded this 50% threshold (FIG. 5A).

Nine of these genes (CUL4B, LTBR, MBD4, MCM3, NHEJ1, PRDX4, PTTG1, SFHM1, and TMEM30A) were then examined in the oxaliplatin-resistant SW480 colorectal tumor cell line (Rixe, O., et al., Oxaliplatin, tetraplatin, cisplatin, and carboplatin: spectrum of activity in drug-resistant cell lines and in the cell lines of the National Cancer Institute's Anticancer Drug Screen panel. Biochem Pharmacol, 1996. 52(12): p. 1855-65). Silencing of each of these 9 genes, all of which conferred increased sensitivity to the HCT 116 tumor cell line, also increased sensitivity of the SW480 tumor cell line to oxaliplatin (FIG. 5B).

### Example 3

To independently test whether the expression of the identified genes relates to tumor cell sensitivity to oxaliplatin, the effects of overexpression of two genes, LTBR and TMEM30A, on response to oxaliplatin were assayed.

Full-length LTBR and TMEM30A open reading frames were cloned into pCMV-XL4 (Origene, Rockville, Md.) and validated by sequencing. Transfection was performed using Turbofectin 8.0® (Origene, Rockville, Md.) in a 96-well format as per manufacturer's instructions using 100 ng cDNA per well. Following a 4 hr incubation, 10 μl per well of an 11-point, 2-fold serial dilution of oxaliplatin (50 μM maximum) was then added. Assays were performed in triplicate, using the empty pCMV-XL4 vector as negative control, with biological replicates. Cell viability was measured 72 h later using the CellTiter 96® AQueous One Solution Cell Proliferation Assay (Promega, Madison, Wis.), and IC₅₀ values calculated using Prism 5.0® (GraphPad, La Jolla, Calif.). Overexpression of cDNA was validated by qRT-PCR using the High-Capacity cDNA Reverse Transcription Kit® (Life Technologies, Carlsbad, Calif.) and qPCR using the 7900 HT Fast Real-Time PCR System® (Life Technologies, Carlsbad, Calif.) with gene-specific primers (ABI, Carlsbad, Calif.).

Transient overexpression of full-length LTBR or TMEM30A (validated by qPCR; FIG. 4B) increased the IC₅₀ of oxaliplatin >2-fold (FIG. 5C), significantly increasing the resistance of the HCT 116 cell line to oxaliplatin, as predicted by the results with siRNA silencing.

### Example 4

To begin to address the cellular mechanisms responsible for modulated cell sensitivity to oxaliplatin, it was asked if siRNA silencing of the identified genes altered the amount of DNA damage acquired by tumor cells treated with oxaliplatin. DNA damage was assessed by quantification of apurinic/apyrimidinic (AP) sites (BioVision, Mountain View, Calif.) following manufacturer's instruction.

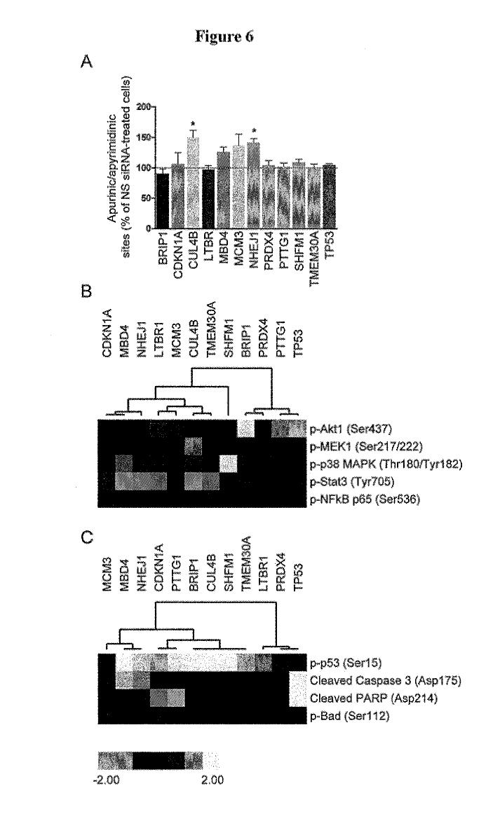

Platinum-DNA adducts formed upon exposure to platinum-based chemotherapies are thought to be primarily removed through the nucleotide excision repair pathway (NER). Using the in vitro assay that measures the number of apurinic/apyrimidinic sites on the DNA of oxaliplatin-treated cells, it was found that siRNA-silencing of CUL4B and NHEJ1, both with known roles in the repair of DNA damage via the NER (Guerrero-Santoro, J., et al., The cullin 4B-based UV-damaged DNA-binding protein ligase binds to UV-damaged chromatin and ubiquitinates histone H2A. Cancer Res, 2008. 68(13): p. 5014-22; Valencia, M., et al., NEJ1 controls non-homologous end joining in Saccharomyces cerevisiae. Nature, 2001. 414(6864): p. 666-9) significantly increased the amount of DNA damage relative to oxaliplatin-treated control cells (FIG. 6A). siRNA silencing of two other genes with known roles in DNA replication and repair, MBD4 and MCM3 (Riccio, A., et al., The DNA repair gene MBD4 (MEDI) is mutated in human carcinomas with microsatellite instability. Nat Genet, 1999. 23(3): p. 266-8; Madine, M. A., et al., MCM3 complex required for cell cycle regulation of DNA replication in vertebrate cells. Nature, 1995. 375(6530): p. 421-4) also increased the amount of DNA damage accumulated upon treatment with oxaliplatin (FIG. 6A), although the increase did not reach statistical significance (P<0.05).

Second, alterations in the phosphorylation of signaling nodes of several pathways whose activity may contribute significantly to changes in cell proliferation were studied, including the mitogen-activated protein kinase cascade, JAK/STAT, and NFκB pathways. To this end, phosphorylation status of AKT1 (Ser437), MEK1 (Ser217/222), p38 MAPK (Thr180/Tyr182), STAT3 (Tyr705), and NFκB p65 (Ser536), was determined using the PathScan Signaling Nodes Multi-Target Sandwich ELISA® (Cell Signaling Technology, Danvers, Mass.) as per manufacturer's instructions. In addition, the phosphorylation status of p53 (Ser15), Bad (Ser112), PARP (Asp214), and cleavage status of Caspase-3 were determined using the PathScan Apoptosis Multi-Target Sandwich ELISA® (Cell Signaling Technology, Danvers, Mass.) following manufacturer's instructions. Raw signal intensity was normalized to either total Akt or Bad protein levels. Assays were performed in duplicate, and the log₂ fold-change (OD₄₅₀ siRNA-treated cells/OD₄₅₀ non-silencing siRNA-treated cells), following median normalization, was converted into a heatmap using Java TreeView.

Quantitative analyses to determine the activity of p-Akt1, p-Mek1, p-p38 MAPK, p-Stat3, and p-NFκB p65 were performed. Hierarchical clustering of phosphorylation levels (relative to control cells) revealed diverse and non-overlapping clusters of pathway signaling following siRNA silencing of the 12 selected genes of Example 2, with the noticeable exception of pNFκB p65, suggesting that distinct cellular mechanisms for each gene are likely responsible for altered cell survival (FIG. 6B). Similarly, when the activities of several gene regulators of apoptosis were probed, including p-p53, p-Bad, cleaved caspase 3 and cleaved PARP, distinct clusters of pathway activity were observed, suggesting that upon siRNA silencing of the genes, both caspase-dependent and caspase-independent pathways regulating changes in apoptosis and/or cell death are modulated in response to DNA damage upon treatment with oxaliplatin (FIG. 6C).

### Example 5

The effects that siRNA silencing of the 12 genes of Example 2 would have on cell cycle were also evaluated.

Transfections were performed as described in Example 2, using six-well plates (5×10⁵ cells/well). Cells were collected by gentle trypsinization, followed by centrifugation at 500 rpm for 5 min, fixed with 70% ethanol at −20° C., washed with PBS, and re-suspended in 0.5 ml of PBS containing propidium iodide (10 μg/ml). After a final incubation at 37° C. for 30 min with RNase A (Sigma, St. Louis, Mo.), cells were analyzed by flow cytometry using a LSR II flow cytometer (Becton Dickinson, Franklin Lakes, N. J.) at ˜200 events/sec using the DNA QC Particles Kit® following manufacturer's instructions (Becton. Dickinson, Franklin Lakes, N. J.). Data were analyzed using FlowJo software (Tree Star, Ashland, Oreg.).

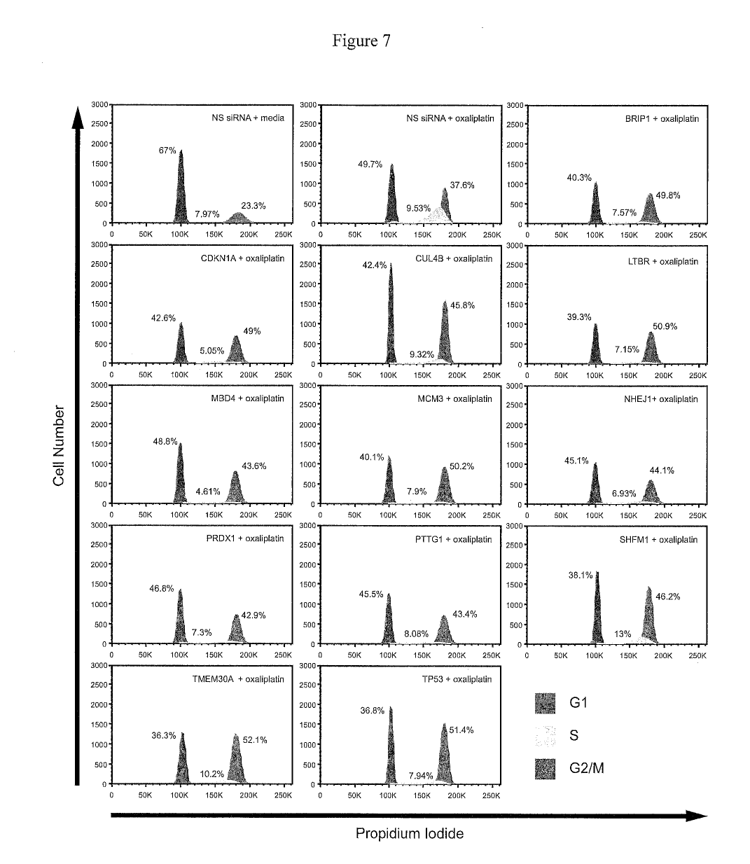

Cell cycle analysis indicates that upon treatment with oxaliplatin, all siRNA-treated cells, including those with increased siRNA-mediated resistance to oxaliplatin (CDKN1A and p53), exhibited a significant decrease in the percentage of cells in G1 with a concomitant increase in the percentage of cells in G2/M as compared to control cells (FIG. 7). This is consistent with previous observations that G2/M arrest facilitates platinum-mediated cell death (Sorenson, C. M. and A. Eastman, “Influence of cis-diamminedichloroplatinum(II) on DNA Synthesis sand Cell Cycle Progression in Excision Repair Proficient and Deficient Chinese Hamster Ovary Cells,” Cancer Res., 1988. 48(23): p. 6703-7; Sorenson, C. M. and A. Eastman, “Mechanism of cis-diamminedichloroplatinum(II)-Induced Cytotoxicity: Role of G2 Arrest and DNA Double-Strand Breaks,” Cancer Res., 1988. 48(16): p. 4484-8), although it is of note that there were no gross differences between oxaliplatin-sensitive and -resistant cells.

### Example 6

To further understand the functional relationships between those genes whose loss of expression altered the sensitivity of tumor cells to oxaliplatin, an extensive bioinformatic analysis was performed using the statistically significant genes validated in the initial screen to identify relevant networks of interacting proteins.

Data were analyzed through the use of Ingenuity® Pathways Analysis (Ingenuity Systems, www.ingenuity.com), PANTHER® (www.panther.org) (Thomas, P. D., et al., PANTHER: a browsable database of gene products organized by biological function, using curated protein family and subfamily classification. Nucleic Acids Res, 2003. 31(1): p. 334-41), or GOTermFinder® (go.princeton.edu/cgi-bin/GOTermFinder) (Boyle, E. L, et al., GO::TermFinder—open source software for accessing Gene Ontology information and finding significantly enriched Gene Ontology terms associated with a list of genes. Bioinformatics, 2004. 20(18): p. 3710-5). Briefly, the functional analysis of siRNA hits identified the biological functions that were most significantly associated with identified genes. The network-associated genes with biological functions in the Ingenuity Pathways Knowledge Base were considered for the analysis. Fischer's exact test was used to calculate the probability that each biological function assigned to that network is due to chance alone.

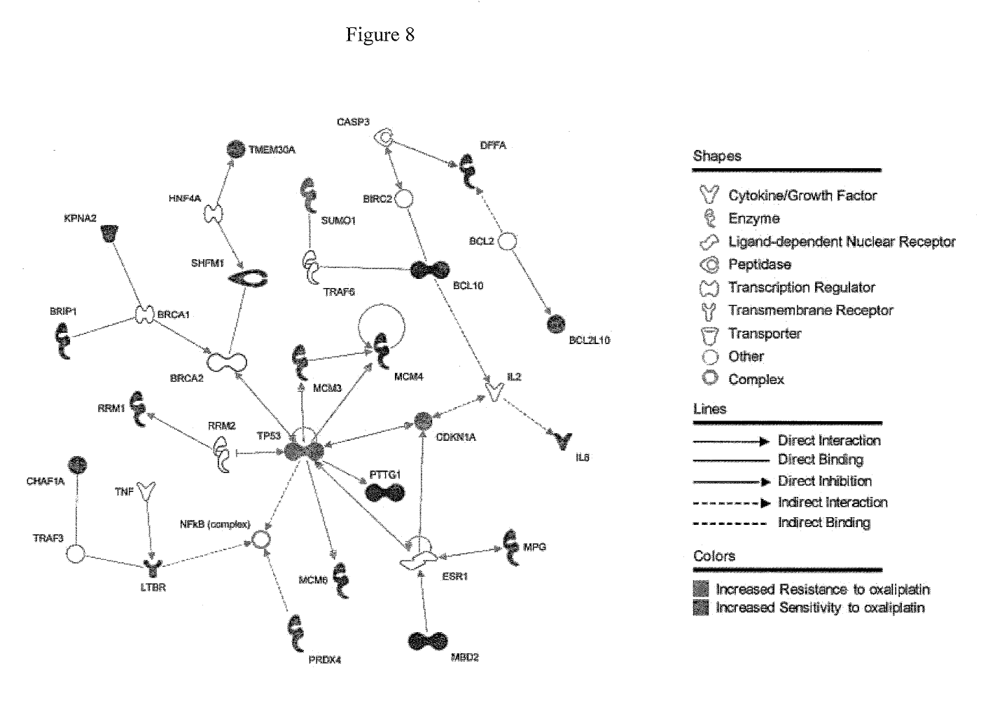

The most significantly enriched interaction network is heavily populated with genes that have roles in DNA replication, recombination, repair and cell cycle progression (FIG. 8). It is, however, of interest that this interaction network contains nodes previously not associated with response to oxaliplatin, which link it to proteins from the canonical (BCL10, TRAF6) and non-canonical NFkB pathways (LTBR, TRAF3, PRDX4) (Perkins, N. D., “Integrating Cell-Signalling Pathways with NF-kappaB and IKK Function,” Nat Rev Mol Cell Biol, 2007. 8(1): p. 49-62), as well as the estrogen signaling (ESR1, MPG, MDB2), apoptosis (BCL2, BCL2L10, BCL10, DFFA, CASP3, and BIRC2), and BRCA1/2-signaling pathways (BRCA1, BRCA2, SHFM1, and BRIP1).

### Example 7

The genes listed in any of Tables 1-4, as well as any of the gene subsets identified in Examples 1 and Example 2, are studied on tissue samples obtained from human patients with colorectal cancer enrolled in the National Surgical Adjuvant Breast and Bowel Project (NSABP) protocol C-07 (NSABP C-07) phase III clinical trial. See Kuebler J. P. et al., “Oxaliplatin Combined with Weekly Bolus Fluorouracil and Leucovorin as Surgical Adjuvant Chemotherapy for Stage II and III Colon Cancer: Results from NSABP C-07,” J. Clin. Oncol. 25:2198-2204 (2007). An objective of the study is to determine whether there is a significant relationship between the expression of the genes and clinical outcome in the patient who received oxaliplatin after colon resection surgery. Improvement in a clinical endpoint, such as recurrence-free interval (RFI), distant recurrence-free interval (DRFI), overall survival (OS), and disease-free survival (DFS), reflects an increased likelihood of response to treatment with oxaliplatin and a likelihood of a positive response.

Patients in the NSABP C-07 study had either stage II or stage III colorectal cancer and had undergone a potentially curative resection. Their tissue samples were archived, formalin-fixed, and paraffin-embedded prior to treatment. Patients were then randomly assigned to one of the following treatment regimens: (1) FULV: 5-fluorouracil (5-FU) 500 mg/m² intravenous (IV) bolus weekly for 6 weeks plus leucovorin 500 mg/m² IV weekly for 6 weeks during each 8-week cycle for three cycles; or (2) FLOX: the same FULV regimen with oxaliplatin 85 mg/m² IV administered on weeks 1, 3, and 5 of each 8-week cycle for three cycles. Data regarding the clinical responses of each patient are available. See id.

The expression of one or more of the 500 genes, or any gene subset, is quantitatively measured for each patient from the archived, formalin-fixed paraffin-embedded tissue (FPET) samples by RT-PCR. The primers and probes for each of the 500 genes and reference genes may be readily determined by methods known in the art. The Accession Number as given in the Entrez Gene online database by the National Center for Biotechnology Information for each gene is provided in Table 1. For normalization of extraneous effects, cycle threshold (Ct) measurements obtained by RT-PCR are normalized relative to the mean expression of a set of reference genes.

For each of the genes, the Cox proportional hazard model is used to examine the relationship between gene expression and recurrence-free interval (RFI). The likelihood ratio is used as a test of statistical significance. The method of Benjamini and Hochberg (Benajmini™, Y. and Hochberg, Y. (1995), Controlling the False Discovery Rate: A Practical and Powerful Approach to Multiple Testing. J. R. Statist. Soc. B. 57:289-300), as well as resampling and permuation based methods (Tusher, V. G. et al. (2001), Significance Analysis of Microarrays Applied to the Ionizing Radiation Response, PNAS 98:5116-5121; Storey J. D. et al. (2001), Estimating False Discovery Rates Under Dependence, With Applications to DNA Microarrays, Stanford: Stanford University, Department of Statistics, Technical Report 2001-28; Korn E. L. et al. (2001), Controlling the Number of False Discoveries: Application to High-Dimensional Genomic Data, Technical Report 003, National Cancer Institute) may be applied to the resulting set of p-values to estimate false discovery rates. A gene with a p-value of <0.05 is generally considered to have a significant correlation between its gene expression and a positive response to treatment.

A hazard ratio (HR) is calculated for each gene from the Cox proportion hazards regression model for the FLOX group. A gene with HR>1 indicates higher recurrence risk after treatment and therefore, a decreased likelihood of a positive response as gene expression increases. A gene with HR<1 indicates lower recurrence risk after treatment and therefore, an increased likelihood of a positive response as gene expression increases. Additionally, the hazard ratios provide an assessment of the contribution of the instantaneous risk of recurrence at time t conditional on a recurrence not occurring by time t. For an individual with gene expression measurement X, the instantaneous risk of recurrence at time t, λ(t|X) is given by the relationship λ(t|X)=λₒ(t)·exp[β·X] where λₒ(t) is the baseline hazard at time t and p is the log hazard ration (β=ln [HR]). Furthermore, the survivor function at time t is given by S(t|X)=Sₒ(t)^{exP[β·X]}, where Sₒ(t) is the baseline survivor function at time t. Consequently, the risk of recurrence at time t for a patient with a gene expression measurement of X is given by 1−S(t|X). In this way, an individual patient's estimated risk of recurrence may be derived from an observed gene expression measurement.

A hazard ratio may also be calculated for each gene for the FULV group to identify genes whose expression is associated specifically with response to oxaliplatin. A test can be performed to evaluate whether the HR associated with gene expression in the FULV group (received only 5-FU and leucovorin) is sufficiently different from the HR associated with gene expression in the FLOX group (received oxaliplatin in addition to 5-FU and leucovorin).

Accordingly, increased expression level of the one or more genes selected from the group ATP6V0C, BCL10, BCL2L10, BFAR, BRIP1, CARD6, CCND1, CDC20, CDC25A, CFLAR, CHAF1A, CRADD, CUL4B, DFFA, E2F2, E2F4, E2F6, GADD45B, HMG20B, IL8, LTBR, MBD2, MBD3, MBD4, MCM3, MCM4, MCM6, MGST3, MPG, MRPL3, MSH4, NHEJ1, OGT, PAICS, PPP2R5C, PRDX4, PTTG1, RAD51L1, RARA, RBM4, RECQL, RRM1, SHFM1, SPO11, TMEM30A, UBE2A, UBE2S, XAB2, and XRCC2 is negatively correlated with a likelihood that a patient with colorectal cancer will exhibit a positive response to treatment comprising oxaliplatin, and increased expression level of one or more genes selected from ABL1, APAF1, BAX, CARD4, CASP5, CCT5, CDKN1A, CDKN3, CIDEA, CRIP2, CUL1, CYP1A2, DNMT1, ERCC4, FANCE, GSTT1, GSTZ1, GTF2H5, KPNA2, MRPS12, MSH5, NFKB1, PTEN, SMARCA4, SND1, SOX4, SUMO1, TARS, TNFRSF10A, TNFSF8, TP53, XPC, and XRCC3 is positively correlated with a likelihood that a patient with colorectal cancer will exhibit a positive response to treatment comprising oxaliplatin.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Table 1

TABLE 1

| Symbol | Entrez ID | GeneBank | Description | Exemplary Pathway |
|---|---|---|---|---|
| BAG4 | 9530 | NM_004874 | BCL2-associated athanogene 4 | Apoptosis |
| BAK1 | 578 | NM_001188 | BCL2-antagonist/killer 1 | Apoptosis |
| BAX | 581 | NM_004324 | BCL2-associated X protein | Drug Resistance |
| BCCIP | 56647 | NM_016567 | BRCA2 and CDKN1A interacting protein | Cell Cycle |
| BCL10 | 8915 | NM_003921 | B-cell CLL/lymphoma 10 | Apoptosis |
| BCL2 | 596 | NM_000633 | B-cell CLL/lymphoma 2 | Drug Resistance |
| BCL2A1 | 597 | NM_004049 | BCL2-related protein A1 | Apoptosis |
| BCL2L1 | 598 | NM_138578 | BCL2-like 1 | Apoptosis |
| BCL2L10 | 10017 | NM_020396 | BCL2-like 10 (apoptosis facilitator) | Apoptosis |
| BCL2L11 | 10018 | NM_006538 | BCL2-like 11 (apoptosis facilitator) | Apoptosis |
| BCL2L2 | 599 | NM_004050 | BCL2-like 2 | Apoptosis |
| BCLAF1 | 9774 | NM_014739 | BCL2-associated transcription factor 1 | Apoptosis |
| BFAR | 51283 | NM_016561 | Bifunctional apoptosis regulator | Apoptosis |
| BGN | 633 | BC004244 | Biglycan | Colon ODX |
| BID | 637 | NM_001196 | BH3 interacting domain death agonist | Apoptosis |
| BIK | 638 | NM_001197 | BCL2-interacting killer (apoptosis-inducing) | Apoptosis |
| BIRC2 | 329 | NM_001166 | Baculoviral IAP repeat-containing 2 | Apoptosis |
| BIRC3 | 330 | NM_001165 | Baculoviral IAP repeat-containing 3 | Apoptosis |
| BIRC5 | 332 | NM_001168 | Baculoviral IAP repeat-containing 5 (survivin) | p53 Pathway |
| BIRC6 | 57448 | NM_016252 | Baculoviral IAP repeat-containing 6 (apollon) | Apoptosis |
| BIRC8 | 112401 | NM_033341 | Baculoviral IAP repeat-containing 8 | Apoptosis |
| BLM | 641 | NM_000057 | Bloom syndrome | DNA Damage Repair |
| BLMH | 642 | NM_000386 | Bleomycin hydrolase | Drug Resistance |
| BNIP1 | 662 | NM_001205 | BCL2/adenovirus E1B 19kDa interacting protein 1 | Apoptosis |
| BNIP2 | 663 | NM_004330 | BCL2/adenovirus E1B 19kDa interacting protein 2 | Apoptosis |
| BNIP3 | 664 | NM_004052 | BCL2/adenovirus E1B 19kDa interacting protein 3 | Apoptosis |
| BNIP3L | 665 | NM_004331 | BCL2/adenovirus E1B 19kDa interacting protein 3-like | Apoptosis |
| BRAF | 673 | NM_004333 | V-raf murine sarcoma viral oncogene homolog B1 | Apoptosis |
| BRCA1 | 672 | NM_007294 | Breast cancer 1, early onset | p53 Pathway |
| BRCA2 | 675 | NM_000059 | Breast cancer 2, early onset | p53 Pathway |
| BRIP1 | 83990 | AF360549 | BRCA1 interacting protein C-terminal helicase 1 | DNA Damage Repair |
| BTG2 | 7832 | NM_006763 | BTG family, member 2 | p53 Pathway |
| C13orf15 | 28984 | NM_014059 | Chromosome 13 open reading frame 15 | Cell Cycle |
| C18orf37 | 125476 | NM_001098817 | chromosome 18 open reading frame 37 | DNA Damage Repair |
| CANX | 821 | NM_001746 | calnexin | DNA Damage Repair |
| CARD6 | 84674 | NM_032587 | Caspase recruitment domain family, member 6 | Apoptosis |
| CARD8 | 22900 | NM_014959 | Caspase recruitment domain family, member 8 | Apoptosis |
| CARM1 | 10498 | NM_199141 | coactivator-associated arginine methyltransferase 1 | DNA Damage Repair |
| CASP1 | 834 | NM_033292 | Caspase 1, apoptosis-related cysteine peptidase | Apoptosis |
| | | | (interleukin 1, beta, convertase) | |
| CASP10 | 843 | NM_001230 | Caspase 10, apoptosis-related cysteine peptidase | Apoptosis |
| CASP14 | 23581 | NM_012114 | Caspase 14, apoptosis-related cysteine peptidase | Apoptosis |
| CASP2 | 835 | NM_032982 | Caspase 2, apoptosis-related cysteine peptidase | Apoptosis |
| | | | (neural precursor cell expressed, developmentally | |
| | | | down-regulated 2) | |
| CASP3 | 836 | NM_004346 | Caspase 3, apoptosis-related cysteine peptidase | Apoptosis |
| CASP4 | 837 | NM_001225 | Caspase 4, apoptosis-related cysteine peptidase | Apoptosis |
| CASP5 | 838 | NM_004347 | Caspase 5, apoptosis-related cysteine peptidase | Apoptosis |
| CASP6 | 839 | NM_032992 | Caspase 6, apoptosis-related cysteine peptidase | Apoptosis |
| CASP7 | 840 | NM_001227 | Caspase 7, apoptosis-related cysteine peptidase | Apoptosis |
| CASP8 | 841 | NM_001228 | Caspase 8, apoptosis-related cysteine peptidase | Apoptosis |
| CASP9 | 842 | NM_001229 | Caspase 9, apoptosis-related cysteine peptidase | Apoptosis |
| CBX3 | 11335 | BX647444 | chromobox homolog 3 (HP1 gamma homolog, Drosophila) | DNA Damage Repair |
| CCNA1 | 8900 | NM_003914 | Cyclin A1 | Cell Cycle |
| CCNA2 | 890 | NM_001237 | Cyclin A2 | Cell Cycle |
| CCNB1 | 891 | NM_031966 | Cyclin B1 | Cell Cycle |
| CCNC | 892 | NM_005190 | Cyclin C | Cell Cycle |
| CCND1 | 595 | NM_053056 | Cyclin D1 | Drug Resistance |
| CCND2 | 894 | NM_001759 | Cyclin D2 | Cell Cycle |
| CCNE1 | 898 | NM_001238 | Cyclin E1 | Drug Resistance |
| CCNF | 899 | NM_001761 | Cyclin F | Cell Cycle |
| CCNG1 | 900 | NM_004060 | Cyclin G1 | Cell Cycle |
| CCT4 | 10575 | NM_006430 | chaperonin containing TCP1, subunit 4 (delta) | DNA Damage Repair |
| CCT5 | 22948 | NM_012073 | chaperonin containing TCP1, subunit 5 (epsilon) | DNA Damage Repair |
| CD27 | 939 | NM_001242 | CD27 molecule | Apoptosis |
| CD40 | 958 | NM_001250 | CD40 molecule, TNF receptor superfamily member 5 | Apoptosis |
| CD40LG | 959 | NM_000074 | CD40 ligand (TNF superfamily, member 5, hyper-IgM syndrome) | Apoptosis |
| CDC16 | 8881 | NM_003903 | Cell division cycle 16 homolog (S. cerevisiae) | Cell Cycle |
| CDC2 | 983 | NM_001786 | Cell division cycle 2, G1 to S and G2 to M | p53 Pathway |
| CDC20 | 991 | NM_001255 | Cell division cycle 20 homolog (S. cerevisiae) | Cell Cycle |
| CDC25A | 993 | NM_001789 | Cell division cycle 25 homolog A (S. pombe) | p53 Pathway |
| CDC25C | 995 | NM_001790 | Cell division cycle 25 homolog C (S. pombe) | p53 Pathway |
| CDC34 | 997 | NM_004359 | Cell division cycle 34 homolog (S. cerevisiae) | Cell Cycle |
| CDC37 | 11140 | NM_007065 | Cell division cycle 37 homolog (S. cerevisiae) | Cell Cycle |
| CDC6 | 990 | NM_001254 | Cell division cycle 6 homolog (S. cerevisiae) | Cell Cycle |
| CDC7 | 8317 | NM_003503 | Cell division cycle 7 homolog (S. cerevisiae) | Cell Cycle |
| CDK2 | 1017 | NM_001798 | Cyclin-dependent kinase 2 | Drug Resistance |
| CDK4 | 1019 | NM_000075 | Cyclin-dependent kinase 4 | Drug Resistance |
| CDK7 | 1022 | NM_001799 | cyclin-dependent kinase 7 | NER |
| CDK8 | 1024 | NM_001260 | Cyclin-dependent kinase 8 | Cell Cycle |
| CDKN1A | 1026 | NM_000389 | Cyclin-dependent kinase inhibitor 1A (p21, Cip1) | Drug Resistance |
| CDKN1B | 1027 | NM_004064 | Cyclin-dependent kinase inhibitor 1B (p27, Kip1) | Drug Resistance |
| CDKN1C | 1028 | NM_000076 | Cyclin-dependent kinase inhibitor 1C (p57, Kip2) | Cell Cycle |
| CDKN2A | 1029 | NM_000077 | Cyclin-dependent kinase inhibitor 2A (melanoma, p16, inhibits CDK4) | Drug Resistance |
| CDKN2B | 1030 | NM_004936 | Cyclin-dependent kinase inhibitor 2B (p15, inhibits CDK4) | Cell Cycle |
| CDKN2C | 1031 | NM_078626 | Cyclin-dependent kinase inhibitor 2C (p18, inhibits CDK4) | Cell Cycle |
| CDKN2D | 1032 | NM_001800 | Cyclin-dependent kinase inhibitor 2D (p19, inhibits CDK4) | Drug Resistance |
| CDKN3 | 1033 | BQ056337 | cyclin-dependent kinase inhibitor 3 (CDK2-associated dual | DNA Damage Repair |
| | | | specificity phosphatase) | |
| CETN2 | 1069 | BG567463 | centrin, EF-hand protein, 2 | NER |
| CFLAR | 8837 | NM_003879 | CASP8 and FADD-like apoptosis regulator | Apoptosis |
| CHAF1A | 10036 | NM_005483 | chromatin assembly factor 1, subunit A (p150) | DNA Damage Repair |
| CHEK1 | 1111 | NM_001274 | CHK1 checkpoint homolog (S. pombe) | p53 Pathway |
| CHEK2 | 11200 | NM_007194 | CHK2 checkpoint homolog (S. pombe) | p53 Pathway |
| CIDEA | 1149 | NM_001279 | Cell death-inducing DFFA-like effector a | Apoptosis |
| CIDEB | 27141 | NM_014430 | Cell death-inducing DFFA-like effector b | Apoptosis |
| CKS1B | 1163 | NM_001826 | CDC28 protein kinase regulatory subunit 1B | Cell Cycle |
| CKS2 | 1164 | BQ898943 | CDC28 protein kinase regulatory subunit 2 | DNA Damage Repair |
| CLPTM1L | 81037 | NM_030782 | CLPTM1-like | Drug Resistance |
| COL1A2 | 1278 | J03464 | collagen, type I, alpha 2 | DNA Damage Repair |
| COPB2 | 9276 | AK128561 | coatomer protein complex, subunit beta 2 (beta prime) | DNA Damage Repair |
| CRADD | 8738 | NM_003805 | CASP2 and RIPK1 domain containing adaptor with death domain | Apoptosis |
| CRIP2 | 1397 | AK091845 | cysteine-rich protein 2 | DNA Damage Repair |
| CUL1 | 8454 | NM_003592 | Cullin 1 | Cell Cycle |
| CUL2 | 8453 | NM_003591 | Cullin 2 | Cell Cycle |
| CUL3 | 8452 | NM_003590 | Cullin 3 | Cell Cycle |
| CUL4A | 8451 | NM_003589 | Cullin 4A | Cell Cycle |
| CUL4B | 8450 | NM_003588 | cullin 4B | NER |
| CUL5 | 8065 | NM_003478 | Cullin 5 | Cell Cycle |
| CYP1A2 | 1544 | NM_000761 | Cytochrome P450, family 1, subfamily A, polypeptide 2 | Drug Resistance |
| CYP3A4 | 1576 | NM_017460 | Cytochrome P450, family 3, subfamily A, polypeptide 4 | Drug Resistance |
| DCLRE1A | 9937 | D42045 | DNA cross-link repair 1A (PSO2 homolog, S. cerevisiae) | Apoptosis |
| DCLRE1B | 64858 | NM_022836 | DNA cross-link repair 1B (PSO2 homolog, S. cerevisiae) | DNA Damage Repair |
| DCLRE1C | 64421 | NM_001033858 | DNA cross-link repair 1C (PSO2 homolog, S. cerevisiae) | DNA Damage Repair |
| DDB1 | 1642 | NM_001923 | damage-specific DNA binding protein 1, 127kDa | DNA Damage Repair |
| DDB2 | 1643 | AK123492 | damage-specific DNA binding protein 2, 48kDa | NER |
| DDX11 | 1663 | NM_004399 | DEAD/H (Asp-Glu-Ala-Asp/His) box polypeptide 11 (CHL1-like | NER |
| | | | helicase homolog, S. cerevisiae) | |
| DFFA | 1676 | NM_004401 | DNA fragmentation factor, 45kDa, alpha polypeptide | Cell Cycle |
| DHFR | 1719 | NM_000791 | Dihydrofolate reductase | Apoptosis |
| DIRAS3 | 9077 | NM_004675 | DIRAS family, GTP-binding RAS-like 3 | Drug Resistance |
| DMC1 | 11144 | NM_007068 | DMC1 dosage suppressor of mck1 homolog, meiosis-specific | Cell Cycle |
| | | | homologous recombination (yeast) | |
| DNAJC15 | 29103 | NM_013238.2 | DNAJC15 DnaJ (Hsp40) homolog, subfamily C, member 15 | DNA Damage Repair |
| DNM2 | 1785 | NM_004945 | Dynamin 2 | Cell Cycle |
| DNMT1 | 1786 | NM_001379 | DNA (cytosine-5-)-methyltransferase 1 | p53 Pathway |
| DNMT3A | 1788 | AB208833 | DNA (cytosine-5-)-methyltransferase 3 alpha | DNA Damage Repair |
| DNMT3B | 1789 | In multiple clusters | DNA (cytosine-5-)-methyltransferase 3 beta | DNA Damage Repair |
| DOT1L | 84444 | NM_032482 | DOT1-like, histone H3 methyltransferase (S. cerevisiae) | DNA Damage Repair |
| DUT | 1854 | NM_001025248 | dUTP pyrophosphatase | DNA Damage Repair |
| DVL3 | 1857 | D86963 | dishevelled, dsh homolog 3 (Drosophila) | DNA Damage Repair |
| E2F2 | 1870 | NM_004091 | E2F transcription factor 2 | Cell Cycle |
| E2F4 | 1874 | NM_001950 | E2F transcription factor 4, p107/p130-binding | Cell Cycle |
| E2F5 | 1875 | X86097 | E2F transcription factor 5, p130-binding | DNA Damage Repair |
| E2F6 | 1876 | NM_198256 | E2F transcription factor 6 | Cell Cycle |
| EFNB2 | 1948 | NM_004093 | Ephrin-B2 | Colon ODX |
| EGFR | 1956 | NM_005228 | Epidermal growth factor receptor (erythroblastic leukemia | Drug Resistance |
| | | | viral (v-erb-b) oncogene homolog, avian) | |
| EGR1 | 1958 | NM_001964 | Early growth response 1 | p53 Pathway |
| EHMT1 | 79813 | AB058779 | euchromatic histone-lysine N-methyltransferase 1 | DNA Damage Repair |
| EIF4A3 | 9775 | CR749455 | eukaryotic translation initiation factor 4A, isoform 3 | DNA Damage Repair |
| ELK1 | 2002 | NM_005229 | ELK1, member of ETS oncogene family | Drug Resistance |
| EME1 | 146956 | BC016470 | essential meiotic endonuclease 1 homolog 1 (S. pombe) | DNA Damage Repair |
| ERBB2 | 2064 | NM_004448 | V-erb-b2 erythroblastic leukemia viral oncogene homolog 2, | Drug Resistance |
| | | | neuro/glioblastoma derived oncogene homolog (avian) | |
| ERBB3 | 2065 | NM_001982 | V-erb-b2 erythroblastic leukemia viral oncogene homolog 3 (avian) | Drug Resistance |
| ERBB4 | 2066 | NM_005235 | V-erb-a erythroblastic leukemia viral oncogene homolog 4 (avian) | Drug Resistance |
| ERCC1 | 2067 | AK092039 | excision repair cross-complementing rodent repair | NER |
| | | | deficiency, complementation group 1 | |
| ERCC2 | 2068 | AK092872 | excision repair cross-complementing rodent repair | NER |
| | | | deficiency, complementation group 2 | |
| ERCC3 | 2071 | AK127469 | excision repair cross-complementing rodent repair | NER |
| | | | deficiency, complementation group 3 | |
| ERCC4 | 2072 | NM_005236 | excision repair cross-complementing rodent repair | NER |
| | | | deficiency, complementation group 4 | |
| ERCC5 | 2073 | NM_000123 | excision repair cross-complementing rodent repair | NER |
| | | | deficiency, complementation group 5 | |
| ERCC6 | 2074 | Data not found | excision repair cross-complementing rodent repair | NER |
| | | | deficiency, complementation group 6 | |
| ERCC8 | 1161 | AK226129 | excision repair cross-complementing rodent repair | NER |
| | | | deficiency, complementation group 8 | |
| ESR1 | 2099 | NM_000125 | Estrogen receptor 1 | Drug Resistance |
| ESR2 | 2100 | NM_001437 | Estrogen receptor 2 (ER beta) | Drug Resistance |
| EXO1 | 9156 | NM_130398 | exonuclease 1 | DNA Damage Repair |
| EZH2 | 2146 | AB208895 | enhancer of zeste homolog 2 (Drosophila) | DNA Damage Repair |
| FADD | 8772 | NM_003824 | Fas (TNFRSF6)-associated via death domain | Apoptosis |
| FANCA | 2175 | X99226 | Fanconi anemia, complementation group A | DNA Damage Repair |
| FANCB | 2187 | NM_001018113 | Fanconi anemia, complementation group B | DNA Damage Repair |
| FANCC | 2176 | NM_000136 | Fanconi anemia, complementation group C | DNA Damage Repair |
| FANCD2 | 2177 | BC038666 | Fanconi anemia, complementation group D2 | DNA Damage Repair |
| FANCE | 2178 | BC046359 | Fanconi anemia, complementation group E | DNA Damage Repair |
| FANCF | 2188 | NM_022725 | Fanconi anemia, complementation group F | DNA Damage Repair |
| FANCG | 2189 | AJ007669 | Fanconi anemia, complementation group G | DNA Damage Repair |
| FANCL | 55120 | BC037570 | Fanconi anemia, complementation group L | DNA Damage Repair |
| FANCM | 57697 | NM_020937 | Fanconi anemia, complementation group M | DNA Damage Repair |
| FAP | 2191 | U09278 | fibroblast activation protein, alpha | DNA Damage Repair |
| FAS | 355 | NM_000043 | Fas (TNF receptor superfamily, member 6) | Apoptosis |
| FASLG | 356 | NM_000639 | Fas ligand (TNF superfamily, member 6) | Apoptosis |
| FEN1 | 2237 | NM_004111 | flap structure-specific endonuclease 1 | DNA Damage Repair |
| FGF2 | 2247 | NM_002006 | Fibroblast growth factor 2 (basic) | Drug Resistance |
| FLJ35220 | 284131 | NM_173627 | hypothetical protein FLJ35220 | DNA Damage Repair |
| FOS | 2353 | NM_005252 | V-fos FBJ murine osteosarcoma viral oncogene homolog | Drug Resistance |
| G3BP1 | 10146 | NM_005754 | GTPase activating protein (SH3 domain) binding protein 1 | DNA Damage Repair |
| GADD45A | 1647 | NM_001924 | Growth arrest and DNA-damage-inducible, alpha | Apoptosis |
| GADD45B | 4616 | AF087853 | Growth arrest and DNA-damage-inducible, beta | Colon ODX |
| GGT1 | 2678 | NM_005265 | Gamma-glutamyltransferase 1 | Drug Metabolism |
| GPX1 | 2876 | NM_000581 | Glutathione peroxidase 1 | Drug Metabolism |
| GPX2 | 2877 | NM_002083 | Glutathione peroxidase 2 (gastrointestinal) | Drug Metabolism |
| GPX3 | 2878 | NM_002084 | Glutathione peroxidase 3 (plasma) | Drug Metabolism |
| GPX4 | 2879 | NM_002085 | Glutathione peroxidase 4 (phospholipid hydroperoxidase) | Drug Metabolism |
| GPX5 | 2880 | NM_001509 | Glutathione peroxidase 5 (epididymal androgen-related protein) | Drug Metabolism |
| GSK3A | 2931 | NM_019884 | Glycogen synthase kinase 3 alpha | Drug Resistance |
| GSR | 2936 | NM_000637 | Glutathione reductase | Drug Metabolism |
| GSTA3 | 2940 | NM_000847 | Glutathione S-transferase A3 | Drug Metabolism |
| GSTA4 | 2941 | NM_001512 | Glutathione S-transferase A4 | Drug Metabolism |
| GSTM2 | 2946 | NM_000848 | Glutathione S-transferase M2 (muscle) | Drug Metabolism |
| GSTM3 | 2947 | NM_000849 | Glutathione S-transferase M3 (brain) | Drug Metabolism |
| GSTM5 | 2949 | NM_000851 | Glutathione S-transferase M5 | Drug Metabolism |
| GSTP1 | 2950 | NM_000852 | Glutathione S-transferase pi | Drug Metabolism |
| GSTT1 | 2952 | NM_000853 | Glutathione S-transferase theta 1 | Drug Metabolism |
| GSTZ1 | 2954 | NM_001513 | Glutathione transferase zeta 1 (maleylacetoacetate isomerase) | Drug Metabolism |
| GTF2H1 | 2965 | NM_005316 | general transcription factor IIH, polypeptide 1, 62 kDa | NER |
| GTF2H2 | 2966 | BX647532 | general transcription factor IIH, polypeptide 2, 44 kDa | NER |
| GTF2H3 | 2967 | BC039726 | general transcription factor IIH, polypeptide 3, 34 kDa | NER |
| GTF2H4 | 2968 | NM_001517 | general transcription factor IIH, polypeptide 4, 52 kDa | NER |
| GTF2H5 | 404672 | AK055106 | general transcription factor IIH, polypeptide 5 | NER |
| H2AFX | 3014 | BM917453 | H2A histone family, member X | DNA Damage Repair |
| H2AFZ | 3015 | AK056803 | H2A histone family, member Z | DNA Damage Repair |
| HDAC10 | 83933 | NM_032019 | histone deacetylase 10 | DNA Damage Repair |
| HDAC11 | 79885 | AL834223 | histone deacetylase 11 | DNA Damage Repair |
| HDAC2 | 3066 | NM_001527 | histone deacetylase 2 | DNA Damage Repair |
| HDAC4 | 9759 | NM_006037 | histone deacetylase 4 | DNA Damage Repair |
| HDAC6 | 10013 | BC069243 | histone deacetylase 6 | DNA Damage Repair |
| HEL308 | 113510 | NM_133636 | DNA helicase HEL308 | DNA Damage Repair |
| HERC5 | 51191 | NM_016323 | Hect domain and RLD 5 | Cell Cycle |
| HES1 | 3280 | NM_005524.2 | Hairy and enhancer of split 1, (Drosophila) | Notch Pathway |
| HIF1A | 3091 | NM_001530 | Hypoxia-inducible factor 1, alpha subunit (basic helix- | Drug Resistance |
| | | | loop-helix transcription factor) | |
| HLTF | 6596 | NM_003071 | helicase-like transcription factor | DNA Damage Repair |
| HMG20B | 10362 | NM_006339.2 | HMG20B high-mobility group 20B | DNA Damage Repair |
| HNRPA2B1 | 3181 | NM_031243 | heterogeneous nuclear ribonucleoprotein A2/B1 | Apoptosis |
| HRK | 8739 | NM_003806 | Harakiri, BCL2 interacting protein (contains only BH3 domain) | DNA Damage Repair |
| HSP90B1 | 7184 | AB209534 | heat shock protein 90 kDa beta (Grp94), member 1 | DNA Damage Repair |
| HSPD1 | 3329 | NM_002156 | heat shock 60 kDa protein 1 (chaperonin) | Colon ODX |
| HSPE1 | 3336 | BU517060 | Heat shock 10 kDa protein 1 (chaperonin 10) | DNA Damage Repair |
| HSPE1 | 3336 | BU517060 | heat shock 10 kDa protein 1 (chaperonin 10) | DNA Damage Repair |
| HUS1 | 3364 | CR619988 | HUS1 checkpoint homolog (S. pombe) | DNA Damage Repair |
| IARS | 3376 | NM_013417 | isoleucyl-tRNA synthetase | p53 Pathway |
| IFNB1 | 3456 | NM_002176 | Interferon, beta 1, fibroblast | DNA Damage Repair |
| IFNGR2 | 3460 | NM_005534 | interferon gamma receptor 2 (interferon gamma transducer 1) | Drug Resistance |
| IGF1R | 3480 | NM_000875 | Insulin-like growth factor 1 receptor | Drug Resistance |
| IGF2R | 3482 | NM_000876 | Insulin-like growth factor 2 receptor | p53 Pathway |
| IL6 | 3569 | NM_000600 | Interleukin 6 (interferon, beta 2) | Cell Cycle |
| IL8 | 3576 | NM_000584 | Interleukin 8 | DNA Damage Repair |
| ILF2 | 3608 | BG121872 | interleukin enhancer binding factor 2, 45 kDa | Colon ODX |
| INHBA | 3624 | BX648811 | Inhibin, beta A | p53 Pathway |
| JUN | 3725 | NM_002228 | Jun oncogene | DNA Damage Repair |
| KDELR2 | 11014 | NM_006854 | KDEL (Lys-Asp-Glu-Leu) endoplasmic reticulum | DNA Damage Repair |
| | | | protein retention receptor 2 | |
| KIAA0101 | 9768 | AY358648 | KIAA0101 | Cell Cycle |
| KNTC1 | 9735 | NM_014708 | Kinetochore associated 1 | DNA Damage Repair |
| KPNA2 | 3838 | BC067848 | karyopherin alpha 2 (RAG cohort 1, importin alpha 1) | p53 Pathway |
| KRAS | 3845 | NM_004985 | V-Ki-ras2 Kirsten rat sarcoma viral oncogene homolog | DNA Damage Repair |
| LDHA | 3939 | NM_005566 | lactate dehydrogenase A | NER |
| LIG1 | 3978 | AB208791 | ligase I, DNA, ATP-dependent | DNA Damage Repair |
| LIG3 | 3980 | NM_013975 | ligase III, DNA, ATP-dependent | DNA Damage Repair |
| LIG4 | 3981 | NM_002312 | ligase IV, DNA, ATP-dependent | Apoptosis |
| LTA | 4049 | NM_000595 | Lymphotoxin alpha (TNF superfamily, member 1) | Apoptosis |
| LTBR | 4055 | NM_002342 | Lymphotoxin beta receptor (TNFR superfamily, member 3) | Cell Cycle |
| MAD2L1 | 4085 | NM_002358 | MAD2 mitotic arrest deficient-like 1 (yeast) | DNA Damage Repair |
| MAD2L2 | 10459 | AK094316 | MAD2 mitotic arrest deficient-like 2 (yeast) | DNA Damage Repair |
| MBD1 | 4152 | NM_015846 | methyl-CpG binding domain protein 1 | DNA Damage Repair |
| MBD2 | 8932 | NM_003927 | methyl-CpG binding domain protein 2 | DNA Damage Repair |
| MBD3 | 53615 | NM_003926 | methyl-CpG binding domain protein 3 | DNA Damage Repair |
| MBD4 | 8930 | AF072250 | methyl-CpG binding domain protein 4 | Apoptosis |
| MCL1 | 4170 | NM_021960 | Myeloid cell leukemia sequence 1 (BCL2-related) | Cell Cycle |
| MCM2 | 4171 | NM_004526 | Minichromosome maintenance complex component 2 | DNA Damage Repair |
| MCM3 | 4172 | NM_002388 | minichromosome maintenance complex component 3 | Cell Cycle |
| MCM4 | 4173 | NM_005914 | Minichromosome maintenance complex component 4 | Cell Cycle |
| MCM5 | 4174 | NM_006739 | Minichromosome maintenance complex component 5 | Cell Cycle |
| MCM6 | 4175 | NM_005915 | Minichromosome maintenance complex component 6 | Cell Cycle |
| MCM7 | 4176 | NM_005916 | Minichromosome maintenance complex component 7 | p53 Pathway |
| MDM2 | 4193 | NM_002392 | Mdm2, transformed 3T3 cell double minute 2, p53 | DNA Damage Repair |
| | | | binding protein (mouse) | |
| MECP2 | 4204 | NM_004992 | methyl CpG binding protein 2 (Rett syndrome) | Drug Resistance |
| MET | 4233 | NM_000245 | Met proto-oncogene (hepatocyte growth factor receptor) | DNA Damage Repair |
| MGMT | 4255 | CR618411 | O-6-methylguanine-DNA methyltransferase | Drug Metabolism |
| MGST1 | 4257 | NM_020300 | Microsomal glutathione S-transferase 1 | Drug Metabolism |
| MGST2 | 4258 | NM_002413 | Microsomal glutathione S-transferase 2 | Drug Metabolism |
| MGST3 | 4259 | NM_004528 | Microsomal glutathione S-transferase 3 | Cell Cycle |
| MKI67 | 4288 | NM_002417 | Antigen identified by monoclonal antibody Ki-67 | p53 Pathway |
| MLH1 | 4292 | NM_000249 | MutL homolog 1, colon cancer, nonpolyposis type 2 (E. coli) | DNA Damage Repair |
| MLH3 | 27030 | NM_001040108 | mutL homolog 3 (E. coli) | DNA Damage Repair |
| MLL | 4297 | NM_005933 | myeloid/lymphoid or mixed-lineage leukemia (trithorax homolog, | DNA Damage Repair |
| | | | Drosophila) | |
| MMP9 | 4318 | NM_004994 | matrix metallopeptidase 9 (gelatinase B, 92 kDa gelatinase, 92 kDa | DNA Damage Repair |
| | | | type IV collagenase) | |
| MMS19L | 64210 | NM_022362 | MMS19-like (MET18 homolog, S. cerevisiae) | NER |
| MNAT1 | 4331 | NM_002431 | menage a trois homolog 1, cyclin H assembly factor | DNA Damage Repair |
| MPG | 4350 | BF572325 | N-methylpurine-DNA glycosylase | DNA Damage Repair |
| MRE11A | 4361 | NM_005590 | MRE11 meiotic recombination 11 homolog A (S. cerevisiae) | DNA Damage Repair |
| MRPL3 | 11222 | BM541805 | mitochondrial ribosomal protein L3 | DNA Damage Repair |
| MRPS12 | 6183 | BU149479 | mitochondrial ribosomal protein S12 | p53 Pathway |
| MSH2 | 4436 | NM_000251 | MutS homolog 2, colon cancer, nonpolyposis type 1 (E. coli) | DNA Damage Repair |
| MSH3 | 4437 | NM_002439 | mutS homolog 3 (E. coli) | DNA Damage Repair |
| MSH4 | 4438 | BC033030 | mutS homolog 4 (E. coli) | DNA Damage Repair |
| MSH5 | 4439 | AB209886 | mutS homolog 5 (E. coli) | DNA Damage Repair |
| MSH6 | 2956 | NM_000179 | mutS homolog 6 (E. coli) | Drug Metabolism |
| MT2A | 4502 | NM_005953 | Metallothionein 2A | Drug Metabolism |
| MT3 | 4504 | NM_005954 | Metallothionein 3 | DNA Damage Repair |
| MTHFD2 | 10797 | NM_001040409 | methylenetetrahydrofolate dehydrogenase (NADP+ dependent) 2, | Drug Metabolism |
| | | | methenyltetrahydrofolate cyclohydrolase | |
| MTHFR | 4524 | NM_005957 | 5,10-methylenetetrahydrofolate reductase (NADPH) | DNA Damage Repair |
| MUS81 | 80198 | NM_025128 | MUS81 endonuclease homolog (S. cerevisiae) | DNA Damage Repair |
| MUTYH | 4595 | NM_012222 | mutY homolog (E. coli) | Drug Resistance |
| MVP | 9961 | NM_017458 | Major vault protein | Colon ODX |
| MYBL2 | 4605 | BX647151 | V-myb myeloblastosis viral oncogene homolog (avian)-like 2 | p53 Pathway |
| MYC | 4609 | NM_002467 | V-myc myelocytomatosis viral oncogene homolog (avian) | Apoptosis |
| NAIP | 4671 | NM_004536 | NLR family, apoptosis inhibitory protein | DNA Damage Repair |
| NBN | 4683 | BX640816 | Nibrin | DNA Damage Repair |
| NCBP2 | 22916 | AK093216 | nuclear cap binding protein subunit 2, 20 kDa | Notch Pathway |
| NCSTN | 23385 | NM_015331.2 | Nicastrin | DNA Damage Repair |
| NEIL1 | 79661 | AK097008 | nei endonuclease VIII-like 1 (E. coli) | DNA Damage Repair |
| NEIL2 | 252969 | AK056206 | nei like 2 (E. coli) | DNA Damage Repair |
| NEIL3 | 55247 | NM_018248 | nei endonuclease VIII-like 3 (E. coli) | p53 Pathway |
| NF1 | 4763 | NM_000267 | Neurofibromin 1 (neurofibromatosis, von Recklinghausen | Drug Resistance |
| | | | disease, Watson disease) | |
| NFKB1 | 4790 | NM_003998 | Nuclear factor of kappa light polypeptide gene enhancer | Drug Resistance |
| | | | in B-cells 1 (p105) | |
| NFKB2 | 4791 | NM_002502 | Nuclear factor of kappa light polypeptide gene enhancer | Drug Resistance |
| | | | in B-cells 2 (p49/p100) | |
| NFKBIB | 4793 | NM_002503 | Nuclear factor of kappa light polypeptide gene enhancer | Drug Resistance |
| | | | in B-cells inhibitor, beta | |
| NFKBIE | 4794 | NM_004556 | Nuclear factor of kappa light polypeptide gene enhancer | DNA Damage Repair |
| | | | in B-cells inhibitor, epsilon | |
| NHEJ1 | 79840 | NM_024782 | nonhomologous end-joining factor 1 | DNA Damage Repair |
| NME1 | 4830 | BG114681 | non-metastatic cells 1, protein (NM23A) expressed in | Apoptosis |
| NOD1 | 10392 | NM_006092 | Nucleotide-binding oligomerization domain containing 1 | Apoptosis |
| NOL3 | 8996 | NM_003946 | Nucleolar protein 3 (apoptosis repressor with CARD domain) | DNA Damage Repair |
| NONO | 4841 | NM_007363 | non-POU domain containing, octamer-binding | Notch Pathway |
| NOTCH1 | 4851 | NM_017617 | NOTCH 1 Notch homolog 1, translocation-associated (Drosophila) | DNA Damage Repair |
| NTHL1 | 4913 | BQ067653 | nth endonuclease Ill-like 1 (E. coli) | DNA Damage Repair |
| NUDT1 | 4521 | BM455743 | nudix (nucleoside diphosphate linked moiety X)-type motif 1 | Notch Pathway |
| NUMB | 8650 | NM_001005743.1 | Numb homolog (Drosophila) | DNA Damage Repair |
| NUP205 | 23165 | BC146784 | nucleoporin 205 kDa | DNA Damage Repair |
| OGG1 | 4968 | NM_016819 | 8-oxoguanine DNA glycosylase | DNA Damage Repair |
| OGT | 8473 | AL050366 | O-linked N-acetylglucosamine (GlcNAc) transferase (UDP-N- | p53 Pathway |
| | | | acetylglucosamine:polypeptide-N-acetylglucosaminyl transferase) | |
| P53AIP1 | 63970 | NM_022112 | P53-regulated apoptosis-inducing protein 1 | DNA Damage Repair |
| PAFAH1B3 | 5050 | BM904583 | platelet-activating factor acetylhydrolase, isoform | DNA Damage Repair |
| | | | Ib, gamma subunit 29 kDa | |
| PAICS | 10606 | In multiple clusters | phosphoribosylaminoimidazole carboxylase, | DNA Damage Repair |
| | | | phosphoribosylaminoimidazole succinocarboxamide synthetase | |
| PARP1 | 142 | NM_001618 | poly (ADP-ribose) polymerase family, member 1 | DNA Damage Repair |
| PARP2 | 10038 | AK001980 | poly (ADP-ribose) polymerase family, member 2 | p53 Pathway |
| PCNA | 5111 | NM_182649 | Proliferating cell nuclear antigen | Cell Cycle |
| PKMYT1 | 9088 | NM_182687 | Protein kinase, membrane associated tyrosine/threonine 1 | DNA Damage Repair |
| PMS1 | 5378 | CR749432 | PMS1 postmeiotic segregation increased 1 (S. cerevisiae) | DNA Damage Repair |
| PMS2 | 5395 | NM_000535 | PMS2 postmeiotic segregation increased 2 (S. cerevisiae) | DNA Damage Repair |
| PMS2L3 | 5387 | CR621744 | postmeiotic segregation increased 2-like 3 | DNA Damage Repair |
| POLB | 5423 | CR627365 | polymerase (DNA directed), beta | DNA Damage Repair |
| POLD1 | 5424 | AB209560 | polymerase (DNA directed), delta 1, catalytic subunit 125 kDa | DNA Damage Repair |
| POLD3 | 10714 | NM_006591 | polymerase (DNA-directed), delta 3, accessory subunit | DNA Damage Repair |
| POLE | 5426 | In multiple clusters | polymerase (DNA directed), epsilon | NER |
| POLE3 | 54107 | AK092840 | polymerase (DNA directed), epsilon 3 (p17 subunit) | DNA Damage Repair |
| POLG | 5428 | BC050559 | polymerase (DNA directed), gamma | NER |
| POLH | 5429 | NM_006502 | polymerase (DNA directed), eta | DNA Damage Repair |
| POLI | 11201 | NM_007195 | polymerase (DNA directed) iota | DNA Damage Repair |
| POLK | 51426 | BC041798 | polymerase (DNA directed) kappa | DNA Damage Repair |
| POLL | 27343 | AK128521 | polymerase (DNA directed), lambda | DNA Damage Repair |
| POLM | 27434 | BC026306 | polymerase (DNA directed), mu | DNA Damage Repair |
| POLN | 353497 | AK131239 | polymerase (DNA directed) nu | DNA Damage Repair |
| POLQ | 10721 | AY032677 | polymerase (DNA directed), theta | DNA Damage Repair |
| PPARA | 5465 | NM_005036 | Peroxisome proliferative activated receptor, alpha | DNA Damage Repair |
| PPARD | 5467 | NM_006238 | Peroxisome proliferator-activated receptor delta | Drug Resistance |
| PPARG | 5468 | NM_015869 | Peroxisome proliferator-activated receptor gamma | Drug Resistance |
| PPP2R5C | 5527 | NM_002719 | protein phosphatase 2, regulatory subunit B′, gamma isoform | Drug Resistance |
| PRDX2 | 7001 | BM805899 | peroxiredoxin 2 | DNA Damage Repair |
| PRDX4 | 10549 | CD579519 | peroxiredoxin 4 | DNA Damage Repair |
| PRKDC | 5591 | NM_006904 | protein kinase, DNA-activated, catalytic polypeptide | DNA Damage Repair |
| PRMT1 | 3276 | CR622298 | protein arginine methyltransferase 1 | DNA Damage Repair |
| PSEN1 | 5663 | NM_000021.3 | Presenilin 1 | DNA Damage Repair |
| PSMA1 | 5682 | BM455876 | proteasome (prosome, macropain) subunit, alpha type, 1 | Notch Pathway |
| PSMC4 | 5704 | CR611800 | proteasome (prosome, macropain) 26S subunit, ATPase, 4 | DNA Damage Repair |
| PSME2 | 5721 | In multiple clusters | proteasome (prosome, macropain) activator subunit 2 (PA28 beta) | DNA Damage Repair |
| PTEN | 5728 | NM_000314 | Phosphatase and tensin homolog (mutated in multiple | DNA Damage Repair |
| | | | advanced cancers 1 ) | |
| PTMA | 5757 | BM470466 | prothymosin, alpha (gene sequence 28) | p53 Pathway |
| PTP4A3 | 11156 | NM_007079 | PTP4A3 protein tyrosine phosphatase type IVA, member 3 | DNA Damage Repair |
| PTTG1 | 9232 | NM_004219 | Pituitary tumor-transforming 1 | BMS Data |
| PYCARD | 29108 | NM_013258 | PYD and CARD domain containing | p53 Pathway |
| RAD1 | 5810 | NM_133377 | RAD1 homolog (S. pombe) | Apoptosis |
| RAD17 | 5884 | AF076838 | RAD17 homolog (S. pombe) | DNA Damage Repair |
| RAD18 | 56852 | NM_020165 | RAD18 homolog (S. cerevisiae) | DNA Damage Repair |
| RAD23A | 5886 | BF343783 | RAD23 homolog A (S. cerevisiae) | DNA Damage Repair |
| RAD23B | 5887 | NM_002874 | RAD23 homolog B | DNA Damage Repair |
| RAD50 | 10111 | U63139 | RAD50 homolog (S. cerevisiae) | NER |
| RAD51 | 5888 | NM_002875 | RAD51 homolog (RecA homolog, E. coli) (S. cerevisiae) | DNA Damage Repair |
| RAD51C | 5889 | BC073161 | RAD51 homolog C (S. cerevisiae) | DNA Damage Repair |
| RAD51L1 | 5890 | BX248766 | RAD51-like 1 (S. cerevisiae) | DNA Damage Repair |
| RAD51L3 | 5892 | BX647297 | RAD51-like 3 (S. cerevisiae) | DNA Damage Repair |
| RAD52 | 5893 | NM_134424 | RAD52 homolog (S. cerevisiae) | DNA Damage Repair |
| RAD54B | 25788 | In multiple clusters | RAD54 homolog B (S. cerevisiae) | DNA Damage Repair |
| RAD54L | 8438 | NM_003579 | RAD54-like (S. cerevisiae) | DNA Damage Repair |
| RAD9A | 5883 | NM_004584 | RAD9 homolog A (S. pombe) | DNA Damage Repair |
| RARA | 5914 | NM_000964 | Retinoic acid receptor, alpha | DNA Damage Repair |
| RARB | 5915 | NM_000965 | Retinoic acid receptor, beta | Drug Resistance |
| RARG | 5916 | NM_000966 | Retinoic acid receptor, gamma | Drug Resistance |
| RB1 | 5925 | NM_000321 | Retinoblastoma 1 (including osteosarcoma) | Drug Resistance |
| RBBP8 | 5932 | NM_002894 | Retinoblastoma binding protein 8 | Drug Resistance |
| RBL1 | 5933 | NM_002895 | Retinoblastoma-like 1 (p107) | Cell Cycle |
| RBL2 | 5934 | NM_005611 | Retinoblastoma-like 2 (p130) | Cell Cycle |
| RBM4 | 5936 | AK097592 | RNA binding motif protein 4 | Cell Cycle |
| RBX1 | 9978 | BU 155800 | ring-box 1 | DNA Damage Repair |
| RDM1 | 201299 | NM_145654 | RAD52 motif 1 | NER |
| RECQL | 5965 | L36140 | RecQ protein-like (DNA helicase Q1-like) | DNA Damage Repair |
| RECQL4 | 9401 | BC020496 | RecQ protein-like 4 | DNA Damage Repair |
| RECQL5 | 9400 | NM_004259 | RecQ protein-like 5 | DNA Damage Repair |
| RELA | 5970 | NM_021975 | V-rel reticuloendotheliosis viral oncogene homolog A, nuclear factor | DNA Damage Repair |
| | | | of kappa light polypeptide gene enhancer in B-cells 3, p65 (avian) | |
| RELB | 5971 | NM_006509 | V-rel reticuloendotheliosis viral oncogene homolog B, nuclear factor | p53 Pathway |
| | | | of kappa light polypeptide gene enhancer in B-cells 3 (avian) | |
| REV1 | 51455 | NM_016316 | REV1 homolog (S. cerevisiae) | Drug Resistance |
| REV3L | 5980 | AF078695 | REV3-like, catalytic subunit of DNA polymerase zeta (yeast) | DNA Damage Repair |
| RFC1 | 5981 | NM_002913 | replication factor C (activator 1) 1, 145 kDa | DNA Damage Repair |
| RFC4 | 5984 | NM_002916 | replication factor C (activator 1) 4, 37 kDa | NER |
| RIPK2 | 8767 | NM_003821 | Receptor-interacting serine-threonine kinase 2 | DNA Damage Repair |
| RPA1 | 6117 | NM_002945 | replication protein A1, 70 kDa | Apoptosis |
| RPA2 | 6118 | NM_002946 | replication protein A2, 32 kDa | DNA Damage Repair |
| RPA3 | 6119 | NM_002947 | replication protein A3, 14 kDa | DNA Damage Repair |
| RPA4 | 29935 | U24186 | replication protein A4, 34 kDa | DNA Damage Repair |
| RPL13 | 6137 | AK095954 | ribosomal protein L13 | NER |
| RPL27 | 6155 | BF219474 | ribosomal protein L27 | DNA Damage Repair |
| RPL35 | 11224 | CR622666 | ribosomal protein L35 | DNA Damage Repair |
| RRM1 | 6240 | NM_001033.3 | RRM1 ribonucleotide reductase M1 | DNA Damage Repair |
| RRM2B | 50484 | NM_015713 | ribonucleotide reductase M2 B (TP53 inducible) | DNA Damage Repair |
| RUNX1 | 861 | NM_001001890 | Runt-related transcription factor 1 (acute myeloid | Colon ODX |
| | | | leukemia 1; aml1 oncogene) | |
| RXRA | 6256 | NM_002957 | Retinoid X receptor, alpha | Drug Resistance |
| RXRB | 6257 | NM_021976 | Retinoid X receptor, beta | Drug Resistance |
| SDHC | 6391 | NM_003001 | succinate dehydrogenase complex, subunit C, integral | DNA Damage Repair |
| | | | membrane protein, 15 kDa | |
| SERTAD1 | 29950 | NM_013376 | SERTA domain containing 1 | Cell Cycle |
| SETD7 | 80854 | NM_030648 | SET domain containing (lysine methyltransferase) 7 | DNA Damage Repair |
| SETD8 | 387893 | In multiple clusters | SET domain containing (lysine methyltransferase) 8 | DNA Damage Repair |
| SHFM1 | 7979 | AK094899 | split hand/foot malformation (ectrodactyly) type 1 | DNA Damage Repair |
| SKP2 | 6502 | NM_005983 | S-phase kinase-associated protein 2 (p45) | Cell Cycle |
| SMARCA4 | 6597 | NM_003072 | SWI/SNF related, matrix associated, actin dependent | DNA Damage Repair |
| | | | regulator of chromatin, subfamily a, member 4 | |
| SMUG1 | 23583 | AK091468 | single-strand-selective monofunctional uracil-DNA glycosylase 1 | DNA Damage Repair |
| SND1 | 27044 | NM_014390 | staphylococcal nuclease and tudor domain containing 1 | DNA Damage Repair |
| SNRPE | 6635 | In multiple clusters | small nuclear ribonucleoprotein polypeptide E | DNA Damage Repair |
| SNRPF | 6636 | CD388516 | small nuclear ribonucleoprotein polypeptide F | DNA Damage Repair |
| SOD1 | 6647 | NM_000454 | Superoxide dismutase 1, soluble (amyotrophic lateral | Drug Resistance |
| | | | sclerosis 1 (adult)) | |
| SOX4 | 6659 | NM_003107 | SRY (sex determining region Y)-box 4 | DNA Damage Repair |
| SPO11 | 23626 | AF1 69385 | SPO11 meiotic protein covalently bound to DSB homolog | DNA Damage Repair |
| | | | (S. cerevisiae) | |
| SSBP1 | 6742 | BC008402 | single-stranded DNA binding protein 1 | DNA Damage Repair |
| SSR1 | 6745 | NM_003144 | signal sequence receptor, alpha (translocon-associated protein | DNA Damage Repair |
| | | | alpha) | |
| STAT1 | 6772 | NM_007315 | Signal transducer and activator of transcription 1, 91 kDa | p53 Pathway |
| SULT1E1 | 6783 | NM_005420 | Sulfotransferase family 1E, estrogen-preferring, member 1 | Drug Resistance |
| SUMO1 | 7341 | NM_003352 | SMT3 suppressor of mif two 3 homolog 1 (S. cerevisiae) | Cell Cycle |
| TAP1 | 6890 | NM_000593 | Transporter 1, ATP-binding cassette, sub-family B (MDR/TAP) | Drug Transporters |
| TAP2 | 6891 | NM_000544 | Transporter 2, ATP-binding cassette, sub-family B (MDR/TAP) | Drug Transporters |
| TARS | 6897 | NM_152295 | threonyl-tRNA synthetase | DNA Damage Repair |
| TDG | 6996 | NM_003211 | thymine-DNA glycosylase | DNA Damage Repair |
| TDP1 | 55775 | NM_018319 | tyrosyl-DNA phosphodiesterase 1 | DNA Damage Repair |
| TFDP1 | 7027 | NM_007111 | Transcription factor Dp-1 | Cell Cycle |
| TFDP2 | 7029 | NM_006286 | Transcription factor Dp-2 (E2F dimerization partner 2) | Cell Cycle |
| TGIF1 | 7050 | NM_170695 | TGFB-induced factor homeobox 1 | DNA Damage Repair |
| TMEM30A | 55754 | NM_018247 | transmembrane protein 30A | DNA Damage Repair |
| TNF | 7124 | NM_000594 | Tumor necrosis factor (TNF superfamily, member 2) | Apoptosis |
| TNFRSF10A | 8797 | NM_003844 | Tumor necrosis factor receptor superfamily, member 10a | Apoptosis |
| TNFRSF10B | 8795 | NM_003842 | Tumor necrosis factor receptor superfamily, member 10b | Apoptosis |
| TNFRSF10D | 8793 | NM_003840 | Tumor necrosis factor receptor superfamily, member 10d, | p53 Pathway |
| | | | decoy with truncated death domain | |
| TNFRSF11A | 8792 | NM_003839 | Tumor necrosis factor receptor superfamily, member 11a, | Drug Resistance |
| | | | NFKB activator | |
| TNFRSF11B | 4982 | NM_002546 | Tumor necrosis factor receptor superfamily, member 11b | Apoptosis |
| | | | (osteoprotegerin) | |
| TNFRSF1A | 7132 | NM_001065 | Tumor necrosis factor receptor superfamily, member 1A | Apoptosis |
| TNFRSF21 | 27242 | NM_014452 | Tumor necrosis factor receptor superfamily, member 21 | Apoptosis |
| TNFRSF25 | 8718 | NM_003790 | Tumor necrosis factor receptor superfamily, member 25 | Apoptosis |
| TNFRSF9 | 3604 | NM_001561 | Tumor necrosis factor receptor superfamily, member 9 | Apoptosis |
| TNFSF10 | 8743 | NM_003810 | Tumor necrosis factor (ligand) superfamily, member 10 | Apoptosis |
| TNFSF8 | 944 | NM_001244 | Tumor necrosis factor (ligand) superfamily, member 8 | Apoptosis |
| TOP1 | 7150 | NM_003286 | Topoisomerase (DNA) I | Drug Resistance |
| TOP2A | 7153 | NM_001067 | Topoisomerase (DNA) II alpha 170kDa | Drug Resistance |
| TOP2B | 7155 | NM_001068 | Topoisomerase (DNA) II beta 180kDa | Drug Resistance |
| TP53 | 7157 | NM_000546 | Tumor protein p53 | p53 Pathway |
| TP53 | 7157 | NM_000546 | Tumor protein p53 | p53 Pathway |
| TP53BP1 | 7158 | AF078776 | tumor protein p53 binding protein, 1 | DNA Damage Repair |
| TP53BP2 | 7159 | NM_005426 | Tumor protein p53 binding protein, 2 | Apoptosis |
| TP63 | 8626 | NM_003722 | Tumor protein p63 | p53 Pathway |
| TP73 | 7161 | NM_005427 | Tumor protein p73 | Apoptosis |
| TPMT | 7172 | NM_000367 | Thiopurine S-methyltransferase | Drug Resistance |
| TPX2 | 22974 | NM_012112 | TPX2, microtubule-associated, homolog (Xenopus laevis) | DNA Damage Repair |
| TRADD | 8717 | NM_003789 | TNFRSF1A-associated via death domain | Apoptosis |
| TRAF2 | 7186 | NM_021138 | TNF receptor-associated factor 2 | Apoptosis |
| TRAF3 | 7187 | NM_003300 | TNF receptor-associated factor 3 | Apoptosis |
| TRAF4 | 9618 | NM_004295 | TNF receptor-associated factor 4 | Apoptosis |
| TRDMT1 | 1787 | BX537961 | tRNA aspartic acid methyltransferase 1 | DNA Damage Repair |
| TREX1 | 11277 | In multiple clusters | three prime repair exonuclease 1 | DNA Damage Repair |
| TREX2 | 11219 | NM_080701 | three prime repair exonuclease 2 | DNA Damage Repair |
| TSTA3 | 7264 | AK096752 | tissue specific transplantation antigen P35B | DNA Damage Repair |
| TUBB | 203068 | In multiple clusters | tubulin, beta | DNA Damage Repair |
| UBA1 | 7317 | NM_003334 | Ubiquitin-like modifier activating enzyme 1 | Cell Cycle |
| UBE2A | 7319 | BC042021 | ubiquitin-conjugating enzyme E2A (RAD6 homolog) | DNA Damage Repair |
| UBE2B | 7320 | In multiple clusters | ubiquitin-conjugating enzyme E2B (RAD6 homolog) | DNA Damage Repair |
| UBE2N | 7334 | NM_003348 | ubiquitin-conjugating enzyme E2N (UBC13 homolog, yeast) | DNA Damage Repair |
| UBE2S | 27338 | BM479313 | ubiquitin-conjugating enzyme E2S | DNA Damage Repair |
| UBE2V2 | 7336 | AK094617 | ubiquitin-conjugating enzyme E2 variant 2 | DNA Damage Repair |
| UNG | 7374 | NM_003362 | uracil-DNA glycosylase | DNA Damage Repair |
| VDAC1 | 7416 | NM_003374 | Voltage-dependent anion channel 1 | Drug Transporters |
| VDAC2 | 7417 | NM_003375 | Voltage-dependent anion channel 2 | Drug Transporters |
| XAB2 | 56949 | AK074035 | XPA binding protein 2 | DNA Damage Repair |
| XIAP | 331 | NM_001167 | X-linked inhibitor of apoptosis | Apoptosis |
| XPA | 7507 | AK021661 | xeroderma pigmentosum, complementation group A | NER |
| XPC | 7508 | NM_004628 | xeroderma pigmentosum, complementation group C | NER |
| XRCC1 | 7515 | CR591751 | X-ray repair complementing defective repair in | DNA Damage Repair |
| | | | Chinese hamster cells 1 | |
| XRCC2 | 7516 | CR749256 | X-ray repair complementing defective repair in | DNA Damage Repair |
| | | | Chinese hamster cells 2 | |
| XRCC3 | 7517 | AK124498 | X-ray repair complementing defective repair in | DNA Damage Repair |
| | | | Chinese hamster cells 3 | |
| XRCC4 | 7518 | NM_022550 | X-ray repair complementing defective repair in | DNA Damage Repair |
| | | | Chinese hamster cells 4 | |
| XRCC5 | 7520 | NM_021141 | X-ray repair complementing defective repair in | p53 Pathway |
| | | | Chinese hamster cells 5 (double-strand-break | |
| | | | rejoining; Ku autoantigen, 80 kDa) | |
| XRCC6 | 2547 | BC008343 | X-ray repair complementing defective repair in | DNA Damage Repair |
| | | | Chinese hamster cells 6 (Ku autoantigen, 70 kDa) | |
| ZDHHC17 | 23390 | AB024494 | zinc finger, DHHC-type containing 17 | DNA Damage Repair |

TABLE 2

| | | | | | | | Median Fold | |
|---|---|---|---|---|---|---|---|---|
| | | | | | | | Change IC50 | |
| | Entrez | | | | | | Oxaliplatin | |
| Symbol | ID | Description | | | | | (Log₂) | RSA P-value |
| ATP6V0C | 527 | ATPase, H+ transporting, lysosomal 16 kDa,V0 subunit c | | | | | 0.57 | 3.08E−02 |
| BCL10 | 8915 | B-cell CLL/lymphoma 10 | | | | | 0.65 | 4.76E−03 |
| BCL2L10 | 10017 | BCL2-like 10 (apoptosis facilitator) | | | | | 0.85 | 1.03E−03 |
| BFAR | 51283 | bifunctional apoptosis regulator | | | | | 0.86 | 7.85E−04 |
| BRIP1 | 10549 | BRCA1 interacting protein C-terminal helicase 1 | | | | | 0.72 | 1.65E−03 |
| CARD6 | 84674 | caspase recruitment domain family, member 6 | | | | | 0.86 | 9.33E−04 |
| CCND1 | 595 | cyclin D1 | | | | | 0.61 | 6.18E−04 |
| CDC20 | 991 | cell division cycle 20 homolog (S. cerevisiae) | | | | | 0.70 | 6.74E−03 |
| CDC25A | 993 | cell division cycle 25 homolog A (S. pombe) | | | | | 0.56 | 1.93E−02 |
| CFLAR | 8837 | CASP8 and FADD-like apoptosis regulator | | | | | 0.62 | 1.56E−02 |
| CHAF1A | 10036 | chromatin assembly factor 1, subunit A (p150) | | | | | 0.68 | 2.67E−03 |
| CRADD | 8738 | CASP2 and RIPK1 domain containing adaptor with death domain | | | | | 0.71 | 1.11E−02 |
| CUL4B | 8450 | cullin 4B | | | | | 0.74 | 1.59E−03 |
| DFFA | 1676 | DNA fragmentation factor, 45 kDa, alpha polypeptide | | | | | 0.74 | 1.31E−03 |
| E2F2 | 1870 | E2F transcription factor 2 | | | | | 0.59 | 2.35E−02 |
| E2F4 | 1874 | E2F transcription factor 4, p107/p130-binding | | | | | 0.60 | 3.98E−02 |
| E2F6 | 1876 | E2F transcription factor 6 | | | | | 0.75 | 1.08E−02 |
| GADD45B | 4616 | growth arrest and DNA-damage-inducible, beta | | | | | 0.83 | 1.85E−02 |
| HMG20B | 10362 | high-mobility group 20B | | | | | 1.08 | 1.46E−02 |
| IL8 | 3576 | interleukin 8 | | | | | 0.80 | 1.23E−03 |
| LTBR | 4055 | lymphotoxin beta receptor (TNFR superfamily, member 3) | | | | | 1.56 | 7.65E−05 |
| MBD2 | 8932 | methyl-CpG binding domain protein 2 | | | | | 0.74 | 1.52E−03 |
| MBD3 | 53615 | methyl-CpG binding domain protein 3 | | | | | 0.56 | 3.78E−02 |
| MBD4 | 8930 | methyl-CpG binding domain protein 4 | | | | | 1.10 | 3.01E−04 |
| MCM3 | 4172 | minichromosome maintenance complex component 3 | | | | | 1.17 | 1.31E−04 |
| MCM4 | 4173 | minichromosome maintenance complex component 4 | | | | | 0.62 | 4.80E−03 |
| MCM6 | 4175 | minichromosome maintenance complex component 6 | | | | | 0.66 | 3.08E−03 |
| MGST3 | 4259 | microsomal glutathione S-transferase 3 | | | | | 0.58 | 8.03E−03 |
| MPG | 4350 | N-methylpurine-DNA glycosylase | | | | | 0.99 | 6.72E−04 |
| MRPL3 | 11222 | mitochondrial ribosomal protein L3 | | | | | 0.53 | 9.89E−03 |
| MSH4 | 4438 | mutS homolog 4 (E. coli) | | | | | 0.66 | 2.75E−03 |
| NHEJ1 | 79840 | nonhomologous end-joining factor 1 | | | | | 1.09 | 4.09E−04 |
| OGT | 8473 | O-linked N-acetylglucosamine (GlcNAc) transferase (UDP-N- | | | | | 0.55 | 7.73E−04 |
| | | acetylglucosamine:pol [symbol] | | | | | | |
| PAICS | 10606 | phosphoribosylaminoimidazole carboxylase, phosphoribo- | | | | | 0.51 | 3.40E−02 |
| | | sylaminoimidazole succi [symbol] | | | | | | |
| PPP2R5C | 5527 | protein phosphatase 2, regulatory subunit B′, gamma | | | | | 0.60 | 1.14E−03 |
| PRDX4 | 10549 | peroxiredoxin 4 | | | | | 0.64 | 4.77E−03 |
| PTTG1 | 9232 | pituitary tumor-transforming 1 | | | | | 0.83 | 1.06E−03 |
| RAD51L1 | 5890 | RAD51-like 1 (S. cerevisiae) | | | | | 0.87 | 7.56E−04 |
| RARA | 5914 | retinoic acid receptor, alpha | | | | | 0.64 | 1.19E−02 |
| RBM4 | 5936 | RNA binding motif protein 4 | | | | | 0.70 | 2.00E−02 |
| RECQL | 5965 | RecQ protein-like (DNA helicase Q1-like) | | | | | 0.56 | 5.88E−04 |
| RRM1 | 6240 | ribonucleotide reductase M1 | | | | | 0.76 | 1.28E−03 |
| SHFM1 | 8930 | split hand/foot malformation (ectrodactyly) type 1 | | | | | 1.11 | 1.50E−04 |
| SP011 | 23626 | SPO11 meiotic protein covalently bound to DSB homolog | | | | | 0.70 | 2.53E−02 |
| | | (S. cerevisiae) | | | | | | |
| TMEM30A | 55754 | transmembrane protein 30A | | | | | 1.49 | 9.85E−05 |
| UBE2A | 7319 | ubiquitin-conjugating enzyme E2A (RAD6 homolog) | | | | | 0.53 | 2.26E−04 |
| UBE2S | 27338 | ubiquitin-conjugating enzyme E2S | | | | | 0.50 | 4.83E−02 |
| XAB2 | 56949 | XPA binding protein 2 | | | | | 0.74 | 4.77E−02 |
| XRCC2 | 7516 | X-ray repair complementing defective repair in Chinese | | | | | 0.81 | 5.54E−03 |
| | | hamster cells 2 | | | | | | |
| | | | | | | | Median Fold | |
| | | | | | | | Change IC50 | |
| | | | | | | | Oxaliplatin | |
| | Entrez | | | | | | (Log₂) | |
| Symbol | ID | Description | | | | | from HTS | RSA P-value |
| ABL1 | 25 | c-abl oncogene 1, receptor tyrosine kinase | | | | | −0.33 | 2.51E−02 |
| APAF1 | 317 | apoptotic peptidase activating factor 1 | | | | | −0.34 | 4.61E−02 |
| BAX | 581 | BCL2-associated X protein | | | | | −0.42 | 7.92E−03 |
| CARD4 | 10392 | nucleotide-binding oligomerization domain containing 1 | | | | | −0.44 | 1.63E−03 |
| CASP5 | 838 | caspase 5, apoptosis-related cysteine peptidase | | | | | −0.36 | 1.00E−02 |
| CCT5 | 22948 | chaperonin containing TCP1, subunit 5 (epsilon) | | | | | −0.49 | 4.28E−04 |
| CDKN1A | 1026 | cyclin-dependent kinase inhibitor 1A (p21, Cip1) | | | | | −1.51 | 1.02E−13 |
| CDKN3 | 1033 | cyclin-dependent kinase inhibitor 3 | | | | | −0.30 | 1.21E−02 |
| CIDEA | 1149 | cell death-inducing DFFA-like effector a | | | | | −0.35 | 6.90E−04 |
| CRIP2 | 1397 | cysteine-rich protein 2 | | | | | −0.38 | 5.90E−03 |
| CUL1 | 8454 | cullin 1 | | | | | −0.39 | 8.05E−03 |
| CYP1A2 | 1544 | cytochrome P450, family 1, subfamily A, polypeptide 2 | | | | | −0.29 | 2.12E−03 |
| DNMT1 | 1786 | DNA (cytosine-5-)-methyltransferase 1 | | | | | −0.45 | 1.88E−04 |
| ERCC4 | 2072 | excision repair cross-complementing rodent repair | | | | | −0.37 | 1.61E−03 |
| | | deficiency, complementation g [symbol] | | | | | | |
| FANCE | 2178 | Fanconi anemia, complementation group E | | | | | −0.56 | 2.56E−02 |
| GSTT1 | 2952 | glutathione S-transferase theta 1 | | | | | −0.44 | 4.10E−02 |
| GSTZ1 | 2954 | glutathione transferase zeta 1 | | | | | −0.35 | 1.13E−02 |
| GTF2H5 | 404672 | general transcription factor IIH, polypeptide 5 | | | | | −0.31 | 3.70E−02 |
| KPNA2 | 3838 | karyopherin alpha 2 (RAG cohort 1, importin alpha 1) | | | | | −0.55 | 5.34E−04 |
| MRPS12 | 6183 | mitochondrial ribosomal protein S12 | | | | | −0.28 | 2.40E−03 |
| MSH5 | 4439 | mutS homolog 5 (E. coli) | | | | | −0.72 | 1.10E−02 |
| NFKB1 | 4790 | nuclear factor of kappa light polypeptide gene enhancer | | | | | −0.41 | 5.12E−04 |
| | | in B-cells 1 | | | | | | |
| PTEN | 5728 | phosphatase and tensin homolog | | | | | −0.35 | 3.62E−04 |
| SMARCA4 | 6597 | SWI/SNF related, matrix associated, actin dependent | | | | | −0.29 | 1.66E−02 |
| | | regulator of chromatin, subfa [symbol] | | | | | | |
| SND1 | 27044 | staphylococcal nuclease and tudor domain containing 1 | | | | | −0.31 | 3.87E−03 |
| SOX4 | 6659 | SRY (sex determining region Y)-box 4 | | | | | −0.45 | 5.23E−04 |
| SUMO1 | 7341 | SMT3 suppressor of mif two 3 homolog 1 (S. cerevisiae) | | | | | −0.58 | 2.01E−05 |
| TARS | 6897 | threonyl-tRNA synthetase | | | | | −0.37 | 1.19E−02 |
| TNFRSF10A | 8797 | tumor necrosis factor receptor superfamily, member 10a | | | | | −0.38 | 7.99E−03 |
| TNFSF8 | 944 | tumor necrosis factor (ligand) superfamily, member 8 | | | | | −0.36 | 1.68E−02 |
| TP53 | 7157 | tumor protein p53 | | | | | −1.51 | 2.27E−05 |
| XPC | 7508 | xeroderma pigmentosum, complementation group C | | | | | −0.43 | 4.42E−04 |
| XRCC3 | 7517 | X-ray repair complementing defective repair in Chinese | | | | | −0.38 | 2.12E−03 |
| | | hamster cells 3 | | | | | | |
| | SEQ | | SEQ | | SEQ | | SEQ | |
| | ID | siRNA Sequence 1 | ID | siNA Sequence 2 | ID | siRNA Sequence 3 | ID | siRNA Sequence 4 |
| Symbol | NO. | from HTS | NO. | from HTS | NO. | from HTS | NO. | from HTS |
| ATP6V0C | 1 | CAGCCACAGAATATT | 2 | CTGGATGTTTATTTA | 3 | TAGAATTGTCATTTC | 4 | TCCCAGCTATCTATA |
| | | ATGTAA | | TAAAGA | | TCTTTA | | ACCTTA |
| BCL10 | 5 | CACGTACTGTTTCAC | 6 | GTGCTGAAACTTAGA | 7 | AGGGAATATATCTCT | 8 | ACACAGCGCCATAGT |
| | | GACAAT | | AATATA | | ATTTGA | | AGTTAA |
| BCL2L10 | 9 | ACAGATGTGTGAGAA | 10 | ATGACAGATGTGTGA | 11 | ATGGCTCTTCCTTGA | 12 | CTGCCCAACTGTGAC |
| | | CAAGAA | | GAACAA | | GTGAAA | | CAACTA |
| BFAR | 13 | CCGGGACGAGTGGAA | 14 | TCCGGTGTGCTCACA | 15 | CAGGTCCCTGTTCCT | 16 | CGGGACGAGTGGAAT |
| | | TGATTA | | GCTTTA | | GCTATA | | GATTAA |
| BRIP1 | 17 | CCTGAACTTTACGAT | 18 | AAGATAAACAGTCCA | 19 | CAGGCCCTTGGTAGA | 20 | TAGCATGGCAACAAT |
| | | CCTGAA | | CTTCAA | | TGTATT | | CTCTTA |
| CARD6 | 21 | AACCTTCTCCATGCA | 22 | CCCAATTTGCTTGAA | 23 | CTGCTTATTTGGTGT | 24 | AAGTGTTATATCCCT |
| | | AATCTA | | TGGGAA | | GGTTAA | | AACCAA |
| CCND1 | 25 | AAGGCCAGTATGATT | 26 | CTCCTACGATACGCT | 27 | AGGGTTATCTTAGAT | 28 | ATGCATGTAGTCACT |
| | | TATAAA | | ACTATA | | GTTTCA | | TTATAA |
| CDC20 | 29 | CACCACCATGATGTT | 30 | CTCCCTAAGCTGGAA | 31 | AAGGCATCCGCTGAA | 32 | CAGACATTCACCCAG |
| | | CGGGTA | | CAGCTA | | GACCAA | | CATCAA |
| CDC25A | 33 | AAGGCGCTATTTGGC | 34 | AAGGGTTATCTCTTT | 35 | CAGCTTAGCTAGCAT | 36 | CTGGCCAAATAGCAA |
| | | GCTTCA | | CATACA | | TACTAA | | AGACAA |
| CFLAR | 37 | CACCGACGAGTCTCA | 38 | TCGAGGCATTACAAT | 39 | TTGCCTCAGAGCATA | 40 | CACCTTGTTTCGGAC |
| | | ACTAAA | | CGCGAA | | CCTGAA | | TATAGA |
| CHAF1A | 41 | CACAATAAACTAAAT | 42 | AAGGAAGAAGAGAAA | 43 | CTGCCCTTTAATAAA | 44 | CAGCCATGGATTGCA |
| | | TCTGAA | | CGGTTA | | GCATTA | | AAGATA |
| CRADD | 45 | AGGCAGGTGTCTCAT | 46 | CAGGGTTTCCACTAG | 47 | ATGCGAATTACTATA | 48 | AATGCGAATTACTAT |
| | | ATGTAA | | ACATTA | | TATAAT | | ATATAA |
| CUL4B | 49 | AAGGTGTTAAATACA | 50 | AATGATGATTTCAAA | 51 | AAAGATAAGGTTGAC | 52 | TGGCAGCACTATTGT |
| | | CATGAA | | CATAAA | | CATATA | | AATTAA |
| DFFA | 53 | CCGGAGCATCTCAGC | 54 | TGCCTTGAACTGGGA | 55 | CTGGCAGAGGATGGC | 56 | CAGCATCATCCTCCT |
| | | AAGCAA | | CATAAA | | ACCATA | | ATCAGA |
| E2F2 | 57 | TTGAGACGAGGGATT | 58 | TAGGGACCAGGTAGA | 59 | ACCCATTGGGAATGA | 60 | TCCGTGCTGTTGGCA |
| | | ATTTCA | | CTTTAA | | GTTTAA | | ACTTTA |
| E2F4 | 61 | AGGTATCGGGCTAAT | 62 | AACGAATGGATTCCT | 63 | ACCCGGGAGATTGCT | 64 | GCGGATTTACGACAT |
| | | CGAGAA | | ATATAA | | GACAAA | | TACCAA |
| E2F6 | 65 | CAGGGTCAGACCAGT | 66 | CAGGAGGAACTTTCT | 67 | CAGATCGTCATTGCA | 68 | ACCACTTAGATTACT |
| | | AACAAA | | GACTTA | | GTTAAA | | GAGTAA |
| GADD45B | 69 | GAGGATGACATCGCC | 70 | TCCCAGTTTGCGAAT | 71 | TCGTTGGAGACTGAA | 72 | CTGCTGTGACAACGA |
| | | CTGCAA | | TAATAA | | GAGAAA | | CATCAA |
| HMG20B | 73 | CAGCATCCCTTTAGC | 74 | TCGGCGCTTGCGGAA | 75 | CCAGGAGAAGAAGAT | 76 | CACGGAGAAGATCCA |
| | | TTTCAA | | GATGAA | | CAAGAA | | GGAGAA |
| IL8 | 77 | AACAATTGGGTACCC | 78 | CTGCGCCAACACAGA | 79 | CTGATTGTATGGAAA | 80 | CTGGTTGAAACTTGT |
| | | AGTTAA | | AATTAT | | TATAAA | | TTATTA |
| LTBR | 81 | CCGCCACACGGTCAC | 82 | CCGGCGGGTCTATGA | 83 | AAAGGGAGTCATTAA | 84 | TACATCTACAATGGA |
| | | CTGCAA | | CTATCA | | CAACTA | | CCAGTA |
| MBD2 | 85 | AAGATGATGCCTAGT | 86 | TGGAAAGATGATGCC | 87 | CTCGCGAGTGTAACT | 88 | ACCCTTCAGGTGTTA |
| | | AAATTA | | TAGTAA | | TTCATA | | CTAGAA |
| MBD3 | 89 | CCCGGAGATGGAGCA | 90 | GCCGGTGACCAAGAT | 91 | CCAGACGGCGTCCAT | 92 | CGGGAAGAAGTTCCG |
| | | CGTCTA | | TACCAA | | CTTCAA | | CAGCAA |
| MBD4 | 93 | AAGCTTCTCATCGCT | 94 | CCGCCGAATGACCTC | 95 | AAGAGAATCTGTGTG | 96 | CCGAATGACCTCCGC |
| | | ACTATA | | CGCAAA | | TAATAA | | AAAGAA |
| MCM3 | 97 | CACGATTTGACTTGC | 98 | CGGCAGGTATGACCA | 99 | ATCCAGGTTGAAGGC | 100 | CAGGGAATTTATCAG |
| | | TCTTCA | | GTATAA | | ATTCAA | | AGCAAA |
| MCM4 | 101 | CACATTGATGTCATT | 102 | CTCGACAGCTAGAGT | 103 | CTGCATGGCCTTGAT | 104 | CCAAGCATTTATGAA |
| | | CATTAT | | CATTAA | | GAAGAA | | CATGAA |
| MCM6 | 105 | CTGGAACAATTTAAC | 106 | TACAATGAAGACATA | 107 | CCCAGTGAAGTTGGA | 108 | TCCGGTTACTGAATA |
| | | CAGCAA | | AATCAA | | ACCAAA | | AATCAA |
| MGST3 | 109 | CCAGAACACGTTGGA | 110 | CTGGTGCTGCCAGCT | 111 | ATGGCTGTCCTCTCT | 112 | CAAGATGGCTGTCCT |
| | | AGTGTA | | TTATAA | | AAGGAA | | CTCTAA |
| MPG | 113 | CAACCGAGGCATGTT | 114 | CAGGGTGTTTGTGCC | 115 | CTGGCACAGGATGAA | 116 | CCCGCTTTGCAGATG |
| | | CATGAA | | TCATAA | | GCTGTA | | AAGAAA |
| MRPL3 | 117 | CACATTAAATATATG | 118 | CCGCCGAAACAGACA | 119 | AGGGCATAAATATAT | 120 | GCCGCCGAAACAGAC |
| | | AGTTAA | | GTTAAA | | CATTCA | | AGTTAA |
| MSH4 | 121 | ATGCAGTGAGGTCTA | 122 | TCGCTCATATTAATT | 123 | ATCAATTGTCTTGGA | 124 | AACCATTAACATGAG |
| | | ACATAA | | GATGAA | | TGCCAA | | ATTAGA |
| NHEJ1 | 125 | CTGGAGATCCTCATA | 126 | CTGCAAGGAATCGAT | 127 | CCGCCTCATCCTTCT | 128 | GAGAAGATGATCAAA |
| | | CCTCAA | | AGCCAA | | GCATAA | | CAATAA |
| OGT | 129 | AAGATTAATGTTCTT | 130 | CAGGTAAGTATAAGT | 131 | CCGCACGGCTCTGAA | 132 | TACGCGTGCCATCCA |
| | | CATAAA | | ATTCAA | | ACTTAA | | AATTAA |
| PAICS | 133 | CCCAAGGACTTCTAA | 134 | CTCGACTAACAGGGA | 135 | GCCCAAGGACTTCTA | 136 | CACGTGGAAATCTCC |
| | | CAATAA | | CTATAA | | ACAATA | | GTTATT |
| PPP2R5C | 137 | AACGAGCTGCTTTAA | 138 | CCCATTGGAACAAGT | 139 | CTGCTACTTCAGTAA | 140 | CTGGAAATATTGGGA |
| | | GTGAAA | | AAGAAA | | GAATAA | | AGTATA |
| PRDX4 | 141 | AACCTGGTAGTGAAA | 142 | AAGCAAAGCGAAGAT | 143 | AAGGAGGACTTGGGC | 144 | ACAGCTGTGATCGAT |
| | | CAATAA | | TTCCAA | | CAATAA | | GGAGAA |
| PTTG1 | 145 | AAGACCTGCAATAAT | 146 | CAGAATGGCTACTCT | 147 | TAAAGCATTCTTCAA | 148 | TCAGATGAATGCGGC |
| | | CCAGAA | | GATCTA | | CAGAAA | | TGTTAA |
| RAD51L1 | 149 | CAGAGAGAAGACAGA | 150 | CCCGGCATGGGTAGC | 151 | CACAAGTAGGATCAA | 152 | CCCAGTTATCTTGAC |
| | | TTCTTA | | AAGAAA | | GAACAA | | GAATCA |
| RARA | 153 | TGGATAAAGAATAAA | 154 | CCACATCTTCATCAC | 155 | CTCCACCAAGTGCAT | 156 | CAGCTTCCAGTTAGT |
| | | GTTCTA | | CAGCAA | | CATTAA | | GGATAT |
| RBM4 | 157 | ACCGAGCAATATAAT | 158 | CTCAGGAACCGTGGA | 159 | TACGCCTTACACCAT | 160 | CAGACTTGACCGAGC |
| | | GAGCAA | | CCTTAA | | GAGCTA | | AATATA |
| RECQL | 161 | CAGCTTGAAACTATT | 162 | TTGGAGATATATTCA | 163 | CATGCTGAAATGGTA | 164 | AAGAAAGAACATAAC |
| | | AACGTA | | GAATAA | | AATAAA | | AGAGTA |
| RRM1 | 165 | CTGGTGGGTCTCTAG | 166 | AACGGATATATTGAG | 167 | CTGAGAGTATATAAC | 168 | CCGAGATTTCTCTTA |
| | | AAGCAA | | AATCAA | | AACACA | | CAATTA |
| SHFM1 | 169 | CCGGTAGACTTAGGT | 170 | AAGAAGTGTTGAAGT | 171 | AACCCAGGATGGGAC | 172 | CTGCTTGGATTTATT |
| | | CTGTTA | | AACCTA | | ACTAAA | | TGTGTT |
| SPO11 | 173 | CAGAGTGTACTTACC | 174 | TACATATATTATCTA | 175 | ACAACTAATGTTAAC | 176 | TACCTTCTACGATAC |
| | | TAACAA | | CATCAA | | GCATAA | | AACTAA |
| TMEM30A | 177 | AACGATTTAAAGGTA | 178 | CTCGAGATGATAGTC | 179 | ACCGGATAACACGGC | 180 | ATCGATGGCGATGAA |
| | | CAACAA | | AACTAA | | CTTCAA | | CTATAA |
| UBE2A | 181 | AACACCCTCTATGAA | 182 | AAGCGTGTTTCTGCA | 183 | CCCTAAGTGAATAAA | 184 | ATGGAACATTTAAAC |
| | | ATCAAA | | ATAGTA | | CTCAAT | | TTACAA |
| UBE2S | 185 | CCCGATGGCATCAAG | 186 | TCCCTCCAACTCTGT | 187 | CCGGCCGGCCGCAGC | 188 | CCGCCTGCTCTTGGA |
| | | GTCTTT | | CTCTAA | | CATGAA | | GAACTA |
| XAB2 | 189 | CACGTACAACACGCA | 190 | CCGCGTGTACAAGTC | 191 | CAGCTACGTTTGTAC | 192 | CCGGACCTTGTCTTC |
| | | GGTCAA | | ACTGAA | | ATCAAA | | GAGGAA |
| XRCC2 | 193 | CAGGGTACTACGCAA | 194 | TTGCAACGACACAAA | 195 | AGGGTACTACGCAAG | 196 | CACGATGTATACTTC |
| | | GCCTTA | | CTATAA | | CCTTAA | | CCAAAT |
| ABL1 | 197 | AACACTCTAAGCATA | 198 | ACGCACGGACATCAC | 199 | CCAGTGGAGATAACA | 200 | CTGGGCGAATGTCTT |
| | | ACTAAA | | CATGAA | | CTCTAA | | ATTTAA |
| APAF1 | 201 | AAGGGCAATGGAGAT | 202 | CAGTGAAGGTATGGA | 203 | CCGCATTCTGATGCT | 204 | TAGGCAGAGTATAAA |
| | | AAATTA | | ATATTA | | TCGCAA | | GTATTA |
| BAX | 205 | ATCATCAGATGTGGT | 206 | CAGCTCTGAGCAGAT | 207 | CAGGGTTTCATCCAG | 208 | CCGAGTGGCAGCTGA |
| | | CTATAA | | CATGAA | | GATCGA | | CATGTT |
| CARD4 | 209 | CAGCCTGACAAGGTC | 210 | GCCCGCTCATTTGTT | 211 | AAGGCTGAGTACCAT | 212 | CACCCTGAGTCTTGC |
| | | CGCAAA | | AATAAA | | GGGCTA | | GTCCAA |
| CASP5 | 213 | AAGAATCGCGTGGCT | 214 | TTCGTGATAAACCAC | 215 | TCAGCAGAATCTACA | 216 | ACGTGGCTGGACAAA |
| | | CATCAA | | ATGCTA | | AATATA | | CATCTA |
| CCT5 | 217 | CACTGTAGATGCTAT | 218 | TAGCGTCCTTGTTGA | 219 | CCACTTCTGTGATTA | 220 | CCGCGATAATCGTGT |
| | | AATAAA | | CATAAA | | AGTAAA | | GGTGTA |
| CDKN1A | 221 | ATGATTCTTAGTGAC | 222 | CAGTTTGTGTGTCTT | 223 | CTGGCATTAGAATTA | 224 | CTCTGGCATTAGAAT |
| | | TTTAAA | | AATTAT | | TTTAAA | | TATTTA |
| CDKN3 | 225 | CACAATCAAGATCTG | 226 | TCGGGACAAATTAGC | 227 | CACCAGTGTTATCAA | 228 | CTAGCATAATTTGTA |
| | | TATCAA | | TGCACA | | CTTGAA | | TTGAAA |
| CIDEA | 229 | CGGGTGCTGGATGAC | 230 | GAGAGTCACCTTCGA | 231 | ACGCATTTCATGATC | 232 | CACGCATTTCATGAT |
| | | AAGGAA | | CTTGTA | | TTGGAA | | CTTGGA |
| CRIP2 | 233 | GAGCCTTGTGCTGTC | 234 | CTGGCACAAGTTCTG | 235 | CCCACCTGCCAGTGT | 236 | ACGGTTTGAGGATTG |
| | | AATAAA | | CCTCAA | | TATTTA | | CAGAAA |
| CUL1 | 237 | AACGTAGTTATCAGC | 238 | ACCGACAGCACTCAA | 239 | CTCAGGATTGATACA | 240 | CGGGTTCGAGTACAC |
| | | GATTCA | | ATTAAA | | TTTCAA | | CTCTAA |
| CYP1A2 | 241 | CAGCCTAACTTACAT | 242 | CCAGCCTAACTTACA | 243 | CGCCGATGGCACTGC | 244 | CCCACAGGAGAAGAT |
| | | TCTTAA | | TTCTTA | | CATTAA | | TGTCAA |
| DNMT1 | 245 | CCCATCGGMCCGCG | 246 | TCGCTTATCAACTAA | 247 | TCCCGAGTATGCGCC | 248 | CCCAATGAGACTGAC |
| | | CGAAA | | TGATTT | | CATATT | | ATCAAA |
| ERCC4 | 249 | CAGCACCTCGATGTTT | 250 | CTCGCCGTGTAACAA | 251 | AGCAATGACATTAGT | 252 | CGCAAGAGTATCAGT |
| | | ATAAA | | ATGAAA | | TCCAAA | | GATTTA |
| FANCE | 253 | AACGCCGAGGAGAGCT | 254 | TAGCCTGAGGATAAA | 255 | CTGACTTGAATAATT | 256 | TCGAATCTGGATGAT |
| | | TGTAA | | GGCTGA | | TATCAA | | GCTAAA |
| GSTT1 | 257 | AAGCAGGAATGGCTTG | 258 | CTGATTAAAGGTCAG | 259 | CTGAGGCCTTGTGTC | 260 | CCCGTGGGTGCTGGC |
| | | CTTAA | | CACTTA | | CTTTAA | | TGCCAA |
| GSTZ1 | 261 | CGCGCTGAAATTTGGC | 262 | ACGGTGCCCATCAAT | 263 | CTGAAATTTGGCGTG | 264 | TACCATCAGCTCCAT |
| | | GTGAA | | CTCATA | | AATTAA | | CAACAA |
| GTF2H5 | 265 | ATGGACCATTTAGGAA | 266 | CAGGAGCGAGTGGGT | 267 | CACGTCTTTGTAATA | 268 | TTCCCTTACCCAGAA |
| | | TTATA | | GAATTA | | GCAGAA | | ATGAAA |
| KPNA2 | 269 | ACGAATTGGCATGGTG | 270 | CCGGGCTGGTTTGAT | 271 | ACCAGTGGTGGAACA | 272 | CAGATTCAAGAACAA |
| | | GTGAA | | TCCGAA | | GTTGAA | | GGGAAA |
| MRPS12 | 273 | CAGGACCACTATTAAG | 274 | TTCCATCAGGACCAC | 275 | CTGCTGGGACAAGAC | 276 | CACGTTTACCCGCAA |
| | | CCATA | | TATTAA | | ACTGTA | | GCCGAA |
| MSH5 | 277 | AAGAAAGATATTGTTT | 278 | CACCTTCATGATCGA | 279 | TAG GAAGACTCCCGG | 280 | TTGCCAGACATTAGT |
| | | CTTTA | | CCTCAA | | ATTCTA | | GGATAA |
| NFKB1 | 281 | CTGGGTATACTTCATG | 282 | GACGCCATCTATGAC | 283 | ACCGTGTAAACCAAA | 284 | CGCGGTGACAGGAGA |
| | | TGACA | | AGTAAA | | GCCCTA | | CGTGAA |
| PTEN | 285 | AAGATTTATGATGCAC | 286 | CAATTTGAGATTCTA | 287 | ACGGGAAGACAAGTT | 288 | TCGGCTTCTCCTGAA |
| | | TTATT | | CAGTAA | | CATGTA | | AGGGAA |
| SMARCA4 | 289 | CCCGTGGACTTCAAGA | 290 | CCGCGCTACAACCAG | 291 | TCACTGGATGTCAAA | 292 | CCGCAGTTTGGAGTC |
| | | AGATA | | ATGAAA | | CAGTAA | | ACTGTA |
| SND1 | 293 | ATCCACCGTGTTGCAG | 294 | CAGGCTGAACCTGTG | 295 | ACGGTGGACTACATT | 296 | TCGAAAGAAGCTGAT |
| | | ATATA | | GCGCTA | | AGACCA | | TGGGAA |
| SOX4 | 297 | AAGGACAGACGAAGAG | 298 | CACGGTCAAACTGAA | 299 | TCCTTTCTACTTGTC | 300 | CCGCGAGAAACTTGC |
| | | TTTAA | | ATGGAT | | GCTAAA | | ATTGGA |
| SUMO1 | 301 | CAGTTACCTAATCATG | 302 | CTGAATCAAGGATTT | 303 | CTGAAGTGCCTTCTG | 304 | CAGGTTGAAGTCAAG |
| | | TTGAA | | AATTAA | | AATCAA | | ATGACA |
| TARS | 305 | CACCGTTATTGCTAAA | 306 | GAGGAACAGCGTTTC | 307 | ACACCGTTATTGCTA | 308 | AAGCCGATTGGTGCT |
| | | GTAAA | | CGTAAA | | AAGTAA | | GGTGAA |
| TNFRSF10A | 309 | ATCAAACTTCATGATC | 310 | CCGGGTCCACAAGAC | 311 | CAGGCAATGGACATA | 312 | CAGGAACTTTCCGGA |
| | | AATCA | | CTTCAA | | ATATAT | | ATGACA |
| TNFSF8 | 313 | AAGGACTCTCTCACAC | 314 | ACCCATATCAAGGGT | 315 | TAGGGTGTGGTCACT | 316 | CACTAGGAGGCTGAT |
| | | AGGAA | | GACTAA | | CTCAAT | | CTTGTA |
| TP53 | 317 | CAGCATCTTATCCGAG | 318 | TTGCAGTTAAGGGTT | 319 | TTGGTCGACCTTAGT | 320 | CAGAGTGCATTGTGA |
| | | TGGAA | | AGTTTA | | ACCTAA | | GGGTTA |
| XPC | 321 | CCGGCTGGTATTGTCT | 322 | TAGCAAATGGCTTCT | 323 | TCGGAGGGCGATGAA | 324 | CCAGTGGAGATAGAG |
| | | CTACA | | ATCGAA | | ACGTTT | | ATTGAA |
| XRCC3 | 325 | CAGAATTATTGCTGCA | 326 | GAGACACTTAAGGGA | 327 | CCGCTGTGAATTTGA | 328 | AAGCCAAACTGAAAT |
| | | ATTAA | | AATTAA | | CAGCCA | | CGGTAA |

[symbol]

indicates data missing or illegible when filed

TABLE 3

| | | |
|---|---|---|
| Symbol | Entrez ID | Full Name |

Genes conferring sensitivity to oxaliplatin

| | | |
|---|---|---|
| BCL10 | 8915 | B-cell CLL/lymphoma 10 |
| BCL2L10 | 10017 | BCL2-like 10 (apoptosis facilitator) |
| BFAR | 51283 | bifunctional apoptosis regulator |
| BRIP1 | 83990 | BRCA1 interacting protein C-terminal |
| | | helicase 1 |
| CHAF1A | 10036 | chromatin assembly factor 1, subunit A |
| | | (p150) |
| CUL4B | 8450 | cullin 4B |
| DFFA | 1676 | DNA fragmentation factor, 45kDa, alpha |
| | | polypeptide |
| IL8 | 357.6 | interleukin 8 |
| LTBR | 4055 | Lymphotoxin beta receptor (TNFR super- |
| | | family, member 3) |
| MBD2 | 8932 | methyl-CpG binding domain protein 2 |
| MBD4 | 8930 | methyl-CpG binding domain protein 4 |
| MCM3 | 4172 | minichromosome maintenance complex |
| | | component 3 |
| MCM4 | 4173 | minichromosome maintenance complex |
| | | component 4 |
| MCM6 | 4175 | minichromosome maintenance complex |
| | | component 6 |
| MPG | 4350 | N-methylpurine-DNA glycosylase |
| MSH4 | 4438 | mutS homolog 4 (E. coli) |
| NHEJ1 | 79840 | nonhomologous end-joining factor 1 |
| PRDX4 | 10549 | peroxiredoxin 4 |
| PTTG1 | 9232 | pituitary tumor-transforming 1 |
| RAD51L1 | 5890 | RAD51-like 1 (S. cerevisiae) |
| RRM1 | 6240 | ribonucleotide reductase M1 |
| SHFM1 | 7979 | split hand/foot malformation (ectrodactyly) |
| | | type 1 |
| TMEM30A | 55754 | transmembrane protein 30A |

Genes conferring resistance to oxaliplatin

| | | |
|---|---|---|
| CDKN1A | 1026 | Cyclin-dependent kinase inhibitor 1A |
| | | (p21, Cip1) |
| KPNA2 | 3838 | karyopherin alpha 2 (RAG cohort 1, importin |
| | | alpha 1) |
| SUMO1 | 7341 | SMT3 suppressor of mif two 3 homolog 1 |
| | | (S. cerevisiae) |
| TP53 | 7157 | Tumor protein p53 |

TABLE 4

| | | | Median Fold | | Median Fold |
|---|---|---|---|---|---|
| | | | Change IC50 | | Change IC50 |
| | | | Oxaliplatin | | Oxaliplatin |
| | | | (Log₂) from | | (Log₂) from |
| Symbol | Entrez ID | Description | HTS | RSA P-value | Validation |
| LTBR | 4055 | Lymphotoxin beta receptor (TNFR superfamily, member 3) | 1.56 | 7.85E−04 | 0.83 |
| TMEM30A | 55754 | transmembrane protein 30A | 1.49 | 9.85E−05 | 0.98 |
| MCM3 | 4172 | minichromosome maintenance complex component 3 | 1.17 | 1.31E−03 | 1.53 |
| SHFM1 | 7979 | split hand/foot malformation (ectrodactyly) type 1 | 1.11 | 3.01E−04 | 0.69 |
| MBD4 | 8930 | methyl-CpG binding domain protein 4 | 1.10 | 1.50E−04 | 1.37 |
| NHEJ1 | 79840 | nonhomologous end-joining factor 1 | 1.09 | 6.72E−04 | 1.45 |
| BFAR | 51283 | bifunctional apoptosis regulator | 0.86 | 1.03E−03 | 0.33 |
| PTTG1 | 9232 | pituitary tumor-transforming 1 | 0.83 | 1.59E−03 | 2.93 |
| CUL4B | 8450 | cullin 4B | 0.74 | 2.75E−03 | 1.68 |
| BRIP1 | 83990 | BRCA1 interacting protein C-terminal helicase 1 | 0.72 | 4.09E−04 | 1.63 |
| PRDX4 | 10549 | peroxiredoxin 4 | 0.64 | 1.65E−03 | 1.25 |
| CDKN1A | 1026 | Cyclin-dependent kinase inhibitor 1A (p21, Cip1) | −1.51 | 1.02E−13 | −0.62 |
| TP53 | 7157 | Tumor protein p53 | −1.51 | 2.27E−05 | 0.95 |

TABLE 4

| | SEQ | | SEQ | | SEQ | | SEQ | |
|---|---|---|---|---|---|---|---|---|
| | ID | siRNA Sequence 1 | ID | siRNA Sequence 2 | ID | siRNA Sequence 3 | ID | siRNA Sequence 4 |
| Symbol | NO. | from Validation | NO. | from Validation | NO. | from Validation | NO. | from Validation |
| LTBR | 329 | GAACCAAUUUAUCACCCAU | 330 | CCACAUGUGCCGAGAAUUC | 331 | GCACUGAAGCCGAGCUCAA | 332 | AUACUUCCCUGACUUGGUA |
| TMEM30A | 333 | GCGAUGAACUAUAACGCGA | 334 | CCAUCGUCGUUACGUGAAA | 335 | GCACAGAGGAUGUCGCUAA | 336 | GCGAGAUCGAGAUUGAUUA |
| MCM3 | 337 | CUGAUUGCCUGUAAUGUUA | 338 | GCAGGUAUGACCAGUAUAA | 339 | GACCAUAGAGCGACGUUAU | 340 | CUAACCGGCUUCUGAACAA |
| SHFM1 | 341 | GUUAUAAGAUGGAGACUUC | 342 | AGACUGGGCUGGCUUAGAU | 343 | GUUACGAGCUGAACUAGAG | 344 | CAAUGUAGAGGAUGACUUC |
| MBD4 | 345 | GAAGAUUUGAUGUGUACUU | 346 | GGAACAGAAUGCCGUAAGU | 347 | GAAGAUACCAUCCCACGAA | 348 | UAACUUUACUUCCACUCAU |
| NHEJ1 | 349 | GGGCUACGCUGAUUCGAGA | 350 | GAGGGAGCUAGCAACGUUA | 351 | CCUUCAGAUUCUUCGUAAA | 352 | AGAAAGAGUCCACGGGUAC |
| BFAR | 353 | UAACACAGGCCGAGCGAAU | 354 | GCUACGACAUCCUGGUUAA | 355 | AGAAAUAUGGGAAUGAUCA | 356 | GGACAUCACGGUUUCUCAU |
| PTTG1 | 357 | GCUGUGACAUAGAUAUUUA | 358 | UGGGAGAUCUCAAGUUUCA | 359 | GGGAAUCCAAUCUGUUGCA | 360 | GUUGAAUUGCCACCUGUUU |
| CUL4B | 361 | UAAAUAACCUCCUUGAUGA | 362 | CAGAAGUCAUUAAUUGCUA | 363 | CGGAAAGAGUGCAUCUGUA | 364 | GCUAUUGGCCGACAUAUGU |
| BRIP1 | 365 | AGUCAAGAGUCAUCGAAUA | 366 | GAUAGUAUGGUCAACAAUA | 367 | UAACCCAAGUCGCUAUAUA | 368 | GUGCAAAGCCUGGGAUAUA |
| PRDX4 | 369 | GGACUAUGGUGUAUACCUA | 370 | CGUGGGAAAUACUUGGUUU | 371 | GGAUUCCACUUCUUUCAGA | 372 | GAUGAGACACUACGUUUGG |
| CDKN1A | 373 | CGACUGUGAUGCGCUAAUG | 374 | CCUAAUCCGCCCACAGGAA | 375 | CGUCAGAACCCAUGCGGCA | 376 | AGACCAGCAUGACAGAUUU |
| TP53 | 377 | GAAAUUUGCGUGUGGAGUA | 378 | GUGCAGCUGUGGGUUGAUU | 379 | GCAGUCAGAUCCUAGCGUC | 380 | GGAGAAUAUUUCACCCUUC |

1. A method of predicting a likelihood that a human patient with colorectal cancer will exhibit a positive response to a treatment comprising oxaliplatin, comprising:
a. assaying an expression level of one or more genes selected from the group ABL1, APAF1, ATP6V0C, BAX, BCL10, BCL2L10, BFAR, BRIP1, CARD4, CARD6, CASP5, CCND1, CCT5, CDC20, CDC25A, CDKN1A, CDKN3, CFLAR, CHAF1A, CIDEA, CRADD, CRIP2, CUL1, CUL4B, CYP1A2, DFFA, DNMT1, E2F2, E2F4, E2F6, ERCC4, FANCE, GADD45B, GSTT1, GSTZ1, GTF2H5, HMG20B, IL8, KPNA2, LTBR, MBD2, MBD3, MBD4, MCM3, MCM4, MCM6, MGST3, MPG, MRPL3, MRPS12, MSH4, MSH5, NFKB1, NHEJ1, OGT, PAICS, PPP2R5C, PRDX4, PTEN, PTTG1, RAD51L1, RARA, RBM4, RECQL, RRM1, SHFM1, SMARCA4, SND1, SOX4, SPOT 1, SUMO1, TARS, TMEM30A, TNFRSF10A, TNFSF8, TP53, UBE2A, UBE2S, XAB2, XPC, XRCC2, and XRCC3, in a tumor sample obtained from the patient; and
b. predicting a likelihood that the patient will exhibit a positive response, wherein: increased expression level of the one or more genes selected from the group ATP6V0C, BCL10, BCL2L10, BFAR, BRIP1, CARD6, CCND1, CDC20, CDC25A, CFLAR, CHAF1A, CRADD, CUL4B, DFFA, E2F2, E2F4, E2F6, GADD45B, HMG20B, IL8, LTBR, MBD2, MBD3, MBD4, MCM3, MCM4, MCM6, MGST3, MPG, MRPL3, MSH4, NHEJ1, OGT, PAICS, PPP2R5C, PRDX4, PTTG1, RAD51L1, RARA, RBM4, RECQL, RRM1, SHFM1, SPO11, TMEM30A, UBE2A, UBE2S, XAB2, and XRCC2 is negatively correlated with a likelihood of a positive response to treatment comprising oxaliplatin, and increased expression level of one or more genes selected from ABL1, APAF1, BAX, CARD4, CASP5, CCT5, CDKN1A, CDKN3, CIDEA, CRIP2, CUL1, CYP1A2, DNMT1, ERCC4, FANCE, GSTT1, GSTZ1, GTF2H5, KPNA2, MRPS12, MSH5, NFKB1, PTEN, SMARCA4, SND1, SOX4, SUMO1, TARS, TNFRSF10A, TNFSF8, TP53, XPC, and XRCC3 is positively correlated with a likelihood of a positive response to treatment comprising oxaliplatin.

2. The method of claim 1, wherein the expression level of one or more genes selected from BCL10, BCL2L10, BFAR, BRIP1, CDKN1A, CHAF1A, CUL4B, DFFA, IL8, KPNA2, LTBR, MBD2, MBD4, MCM3, MCM4, MCM6, MPG, MSH4, NHEJ1, PRDX4, PTTG1, RAD51L1, RRM1, SHFM1, SUMO1, TMEM30A, and TP53 is assayed.

3. The method of claim 1, wherein the expression level of the one or more genes is normalized against an expression level of one or more reference genes to obtain a normalized expression level of the one or more genes.

4. The method of claim 1, wherein the expression level of the one or more genes is a level of RNA transcript of the one or more genes.

5. The meth of claim 1, wherein the expression level of the one or more genes is a polypeptide level of the one or more genes.

6. The method of claim 4, wherein the level of RNA transcript of the one or more genes is assayed using reverse transcription polymerase chain reaction (RT-PCR).

7. The method of claim 1, wherein the tumor sample is a biopsy sample.

8. The method of claim 1, wherein the tumor sample is a fixed, wax-embedded tissue sample.

9. The method of claim 1, wherein the treatment further comprises one more or additional anti-cancer agents.

10. The method of claim 9, wherein the one or more additional anti-cancer agents is 5-fluorouracil (5-FU) and leucovorin (LV).

11. The method of claim 1, wherein the colorectal cancer is stage II (Dukes B) or stage III (Dukes C) colorectal cancer.

12. The method of claim 1, further comprising creating a report based on the normalized expression level of the one or more genes.
